(19) United States
(12) Patent Application Publication
(10) Pub. No.: US 2010/0286131 A1
(43) Pub. Date: Nov. 11, 2010

(54) VIRAL POLYMERASE INHIBITORS

(75) Inventors: Pierre L. Beaulieu, Laval (CA); Pasquale Forgione, Laval (CA); Alexandre Gagnon, Laval (CA); Cedrickx Godbout, Laval (CA); Julie Naud, Laval (CA); Martin Poirier, Laval (CA); Jean Rancourt, Laval (CA); Timothy A. Stammers, Laval (CA); Bounkham Thavonekham, Laval (CA)

Correspondence Address: MICHAEL P. MORRIS;BOEHRINGER INGELHEIM USA CORPORATION, 900 RIDGEBURY RD, P O BOX 368, RIDGEFIELD, CT, 06877-0368, US

(73) Assignee: BOEHRINGER INGELHEIM INTERNATIONAL GMBH, Ingelheim (DE)

(21) Appl. No.: 12/671,765

(22) PCT Filed: Jul. 31, 2008
(86) PCT No.: PCT/CA08/01411

§ 371 Date: Mar. 1, 2010

Related U.S. Application Data
(60) Provisional application No. 60/953,820, filed on Aug. 3, 2007.

(51) Int. Cl. A61K 31/341

(52) U.S. Cl. 514/227.8

(57) ABSTRACT

Compounds of formula I:

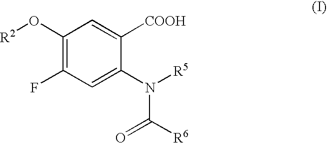

wherein, R², R⁵ and R⁶ are defined herein, are useful as inhibitors of the hepatitis C virus NS5B polymerase.

### RELATED APPLICATIONS

This application claims benefit of U.S. Ser. No. 60/953,820, filed Aug. 3, 2007, which is herein incorporated by reference.

### FIELD OF THE INVENTION

The present invention relates to compounds, compositions and methods for the treatment of hepatitis C virus (HCV) infection. In particular, the present invention provides novel inhibitors of the hepatitis C virus NS5B polymerase, pharmaceutical compositions containing such compounds and methods for using these compounds in the treatment of HCV infection.

### BACKGROUND OF THE INVENTION

It is estimated that at least 130 million persons worldwide are infected with the hepatitis C virus (HCV). Acute HCV infection progresses to chronic infection in a high number of cases, and, in some infected individuals, chronic infection leads to serious liver diseases such as cirrhosis and hepatocellular carcinoma.

Currently, standard treatment of chronic hepatitis C infection involves administration of pegylated interferon-alpha in combination with ribavirin. However, this therapy is not effective in reducing HCV RNA to undetectable levels in many infected patients and is associated with often intolerable side effects such as fever and other influenza-like symptoms, depression, thrombocytopenia and hemolytic anemia. Furthermore, some HCV-infected patients have co-existing conditions which contraindicate this treatment.

Therefore, a need exists for alternative treatments for hepatitis C viral infection. One possible strategy to address this need is the development of effective antiviral agents which inactivate viral or host cell factors which are essential for viral replication.

HCV is an enveloped positive strand RNA virus in the genus Hepacivirus in the Flaviviridae family. The single strand HCV RNA genome is approximately 9500 nucleotides in length and has a single open reading frame (ORF), flanked by 5′ and 3′ non-translated regions. The HCV 5′ non-translated region is 341 nucleotides in length and functions as an internal ribosome entry site for cap-independent translation initiation. The open reading frame encodes a single large polyprotein of about 3000 amino acids which is cleaved at multiple sites by cellular and viral proteases to produce the mature structural and non-structural (NS2, NS3, NS4A, NS4B, NS5A, and NS5B) proteins. The viral NS2/3 protease cleaves at the NS2-NS3 junction; while the viral NS3 protease mediates the cleavages downstream of NS3, at the NS3-NS4A, NS4A-NS4B, NS4B-NS5A and NS5A-NS5B cleavage sites. The NS3 protein also exhibits nucleoside triphosphatase and RNA helicase activities. The NS4A protein acts as a cofactor for the NS3 protease and may also assist in the membrane localization of NS3 and other viral replicase components. Although NS4B and the NS5A phosphoprotein are also likely components of the replicase, their specific roles are unknown. The NS5B protein is the elongation subunit of the HCV replicase possessing RNA-dependent RNA polymerase (RdRp) activity.

The development of new and specific anti-HCV treatments is a high priority, and virus-specific functions essential for replication are the most attractive targets for drug development. The absence of RNA dependent RNA polymerases in mammals, and the fact that this enzyme appears to be essential to viral replication, would suggest that the NS5B polymerase is an ideal target for anti-HCV therapeutics. It has been recently demonstrated that mutations destroying NS5B activity abolish infectivity of RNA in a chimp model (Kolykhalov, A. A.; Mihalik, K.; Feinstone, S. M.; Rice, C. M.; 2000; J. Virol. 74: 2046-2051).

2-amino-5-oxy-benzoic acid inhibitors of the NS5B polymerase of HCV are described in WO2007/087717. In particular compounds according to this invention inhibit RNA synthesis by the RNA dependent RNA polymerase of HCV, especially of the enzyme NS5B encoded by HCV. However, the inhibitors of the invention differ from those described in WO2007/087717 in that they exhibit at least one of the following surprising advantages as compared to their respective non-fluorinated analog, including, in particular:
unexpectedly good activity in a cell-based HCV RNA replication assay; or
improved drug metabolism.

### SUMMARY OF THE INVENTION

The present invention provides a novel series of compounds having good to very good inhibitory activity against HCV polymerase and/or at least one of the following surprising advantages as compared to their respective non-fluorinated analog:
unexpectedly good activity in a cell-based HCV RNA replication assay; or
improved drug metabolism.

Further objects of this invention arise for the one skilled in the art from the following description and the examples.

One aspect of the invention provides compounds of formula (I):

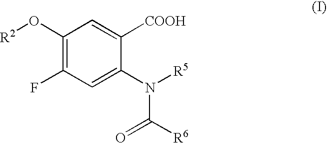

wherein:
R² is aryl or Het, optionally substituted with R²⁰, wherein R²⁰ is 1 to 5 substituents each independently selected from:
   a) halo;
   b) R⁷, wherein R⁷ is selected from H, (C₁₋₆)alkyl, (C₁₋₆)haloalkyl, (C₃₋₇)cycloalkyl, aryl and Het;
      wherein the (C₁₋₆)alkyl and (C₃₋₄cycloalkyl are optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂
      wherein each of the aryl and Het is optionally substituted with 1 to 3 substituents each independently selected from:
      i) halo, cyano, oxo, thioxo, imino, —OH, —O—(C₁₋₆)alkyl, —O—(C₁₋₆)haloalkyl, O—(C₃₋₇)cycloalkyl, (C₃₋₇)cycloalkyl, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄alkyl)(C₃₋₇)cycloalkyl, —NH₂, —NH(C₁₋₄alkyl, —N((C₁₋₄)alkyl)₂, —NH(C₃₋₄cycloalkyl, —N((C₁₋₄alkyl)(C₃₋₇)cycloalkyl or —NH—C(═O)(C₁₋₄alkyl;
      ii) (C₁₋₆)alkyl optionally substituted with —OH, —O—(C₁₋₆)haloalkyl, or —O—(C₁₋₆)alkyl; and
      iii) aryl or Het, wherein each of the aryl and Het is optionally substituted with halo, (C₁₋₆)alkyl or —O—(C₁₋₆)alkyl;
   c) —C(═O)—R⁷, —C(═O)—O—R⁷, —O—R⁷, —S—R⁷, —SO—R⁷, —SO₂—R⁷, —(C₁₋₆)alkylene-R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is as defined above; and wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄alkyl, —NH(C₃₋₄cycloalkyl, —N((C₁₋₄alkyl)(C₃₋₇)cycloalkyl and —N((C₁₋₄)alkyl)₂;
   d) aryl-(C₁₋₆)alkyl- or Het-(C₁₋₆)alkyl-,
      wherein each of the aryl and Het is optionally substituted with 1 to 3 substituents each independently selected from:
      i) halo, cyano, oxo, thioxo, imino, —OH, —O—(C₁₋₆)alkyl, —O—(C₁₋₆)haloalkyl, O—(C₃₋₇)cycloalkyl, (C₃₋₇)cycloalkyl, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl or Het, wherein each of the aryl and Het is optionally substituted with halo, (C₁₋₆)alkyl or —O—(C₁₋₆)alkyl;
      wherein the —(C₁₋₆)alkyl portion of the aryl-(C₁₋₆)alkyl or Het-(C₁₋₆)alkyl is optionally substituted with 1 or 2 substituents each independently selected from —OH, —(C₁₋₆)alkyl, halo, —(C₁₋₆)haloalkyl, (C₃₋₇)cycloalkyl, O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂; and
   e) —N(R⁸)R⁹, —C(═O)—N(R⁸)R⁹, —SO₂—N(R⁸)R⁹, or —(C₁₋₆)alkylene-N(R⁸)R⁹ wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H, (C₁₋₆)alkyl and (C₃₋₇)cycloalkyl; and
      R⁹ is in each instance independently selected from R⁷, —O—(C₁₋₆)alkyl, —(C₁₋₆)alkylene-R⁷, —(C₃₋₇)cycloalkyl-(C₁₋₆)alkyl, —C(═O)—R¹⁰, —C(═O)OR¹⁰ and —C(═O)N(H)R¹⁰;
      wherein R⁷ is as defined above;
      wherein the —(C₁₋₆)alkylene is optionally substituted with 1 or 2 substituents each independently selected from —OH, —(C₁₋₆)alkyl, halo, —(C₁₋₆)haloalkyl, (C₃₋₄cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂
      wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
      wherein R¹⁰ is in each instance independently selected from (C₁₋₆)alkyl, and Het, wherein said Het is optionally substituted with (C₁₋₆)alkyl;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
      wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R⁵ is selected from H, (C₁₋₆)alkyl, (C₃₋₇)cycloalkyl, (C₃₋₇)cycloalkyl-(C₁₋₆)alkyl- and Het; the (C₁₋₆)alkyl and Het each being optionally substituted with 1 to 4 substituents each independently selected from (C₁₋₆)alkyl, —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂ and —SO₂(C₁₋₆)alkyl; and
R⁶ is selected from (C₃₋₇)cycloalkyl and aryl;
   the (C₃₋₇)cycloalkyl and aryl each being optionally substituted with 1 to 5 substituents each independently selected from halo, (C₁₋₆)alkyl, (C₁₋₆)haloalkyl, (C₃₋₇)cycloalkyl, —OH, —SH, —O—(C₁₋₄)alkyl and —S—(C₁₋₄)alkyl;
      wherein Het is a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S; or a salt or ester thereof.

The compounds according to this invention generally show an inhibitory activity against HCV polymerase. In particular compounds according to this invention inhibit RNA synthesis by the RNA dependent RNA polymerase of HCV, especially of the enzyme NS5B encoded by HCV. Furthermore, compounds according to this invention show at least one of the following surprising advantages as compared to their respective non-fluorinated analog:
unexpectedly good activity in a cell-based HCV RNA replication assay; or
improved drug metabolism.

Another aspect of this invention provides compounds of formula (I) showing at least one of the following advantages as compared to their respective non-fluorinated analog:
unexpectedly good activity in a cell-based HCV RNA replication assay; and/or
improved phase I metabolic stability (HLM).

Another aspect of this invention provides a compound of formula (I), or a pharmaceutically acceptable salt or ester thereof, as a medicament.

Still another aspect of this invention provides a pharmaceutical composition comprising a therapeutically effective amount of a compound of formula (I) or a pharmaceutically acceptable salt or ester thereof; and one or more pharmaceutically acceptable carriers.

According to an embodiment of this aspect, the pharmaceutical composition according to this invention additionally comprises at least one other antiviral agent.

The invention also provides the use of a pharmaceutical composition as described hereinabove for the treatment of a hepatitis C viral infection in a mammal having or at risk of having the infection.

A further aspect of the invention involves a method of treating a hepatitis C viral infection in a mammal having or at risk of having the infection, the method comprising administering to the mammal a therapeutically effective amount of a compound of formula (I), a pharmaceutically acceptable salt or ester thereof, or a composition thereof as described hereinabove.

Another aspect of the invention involves a method of treating a hepatitis C viral infection in a mammal having or at risk of having the infection, the method comprising administering to the mammal a therapeutically effective amount of a combination of a compound of formula (I) or a pharmaceutically acceptable salt or ester thereof, and at least one other antiviral agent; or a composition thereof.

Also within the scope of this invention is the use of a compound of formula (I) as described herein, or a pharmaceutically acceptable salt or ester thereof, for the treatment of a hepatitis C viral infection in a mammal having or at risk of having the infection.

Another aspect of this invention provides the use of a compound of formula (I) as described herein, or a pharmaceutically acceptable salt or ester thereof, for the manufacture of a medicament for the treatment of a hepatitis C viral infection in a mammal having or at risk of having the infection.

An additional aspect of this invention refers to an article of manufacture comprising a composition effective to treat a hepatitis C viral infection; and packaging material comprising a label which indicates that the composition can be used to treat infection by the hepatitis C virus; wherein the composition comprises a compound of formula (I) according to this invention or a pharmaceutically acceptable salt or ester thereof.

Still another aspect of this invention relates to a method of inhibiting the replication of hepatitis C virus comprising exposing the virus to an effective amount of the compound of formula (I), or a salt or ester thereof, under conditions where replication of hepatitis C virus is inhibited.

Further included in the scope of the invention is the use of a compound of formula (I), or a salt or ester thereof, to inhibit the replication of hepatitis C virus.

### DETAILED DESCRIPTION OF THE INVENTION

### Definitions

As used herein, the following definitions apply unless otherwise noted:

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “(C₁₋ₙ)alkyl” as used herein, wherein n is an integer, either alone or in combination with another radical, is intended to mean acyclic, straight or branched chain alkyl radicals containing from 1 to n carbon atoms. “(C₁₋₆)alkyl” includes, but is not limited to, methyl, ethyl, propyl (n-propyl), butyl (n-butyl), 1-methylethyl (iso-propyl), 1-methylpropyl (sec-butyl), 2-methylpropyl (iso-butyl), 1,1-dimethylethyl (tert-butyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

The term “(C₁₋ₙ)alkylene” as used herein, wherein n is an integer, either alone or in combination with another radical, is intended to mean acyclic, straight or branched chain divalent alkyl radicals containing from 1 to n carbon atoms. “(C₁₋₆)alkylene” includes, but is not limited to, —CH₂—, —CH₂CH₂—,

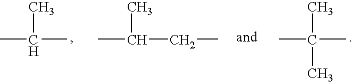

The term “(C₃₋ₘ)cycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl.

The term “(C₃₋ₘ)cycloalkyl-(C₁₋ₙ)alkyl-” as used herein, wherein n and m are both integers, either alone or in combination with another radical, is intended to mean an alkyl radical having 1 to n carbon atoms as defined above which is itself substituted with a cycloalkyl radical containing from 3 to m carbon atoms as defined above. Examples of (C₃₋₇)cycloalkyl-(C₁₋₆)alkyl- include, but are not limited to, cyclopropylmethyl, cyclobutylmethyl, cyclopentylmethyl, cyclohexylmethyl, 1-cyclopropylethyl, 2-cyclopropylethyl, 1-cyclobutylethyl, 2-cyclobutylethyl, 1-cyclopentylethyl, 2-cyclopentylethyl, 1-cyclohexylethyl and 2-cyclohexylethyl. When a (C₃₋ₘ)cycloalkyl-(C₁₋ₙ)alkyl- group is substituted, it is understood that substituents may be attached to either the cycloalkyl or the alkyl portion thereof or both, unless specified otherwise.

The term “aryl” as used herein, either alone or in combination with another radical, is intended to mean a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, indenyl, 1-naphthyl, 2-naphthyl, tetrahydronaphthyl and dihydronaphthyl.

The term “aryl-(C₁₋ₙ)alkyl-” as used herein, wherein n is an integer, either alone or in combination with another radical, is intended to mean an alkyl radical having 1 to n carbon atoms as defined above which is itself substituted with an aryl radical as defined above. Examples of aryl-(C₁₋ₙ)alkyl- include, but are not limited to, phenylmethyl (benzyl), 1-phenylethyl, 2-phenylethyl and phenylpropyl. When an aryl-(C₁₋ₙ)alkyl- group is substituted, it is understood that substituents may be attached to either the aryl or the alkyl portion thereof or both, unless specified otherwise.

The term “Het” as used herein, either alone or in combination with another radical, is intended to mean a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S, unless specified otherwise. When a Het group is substituted, it is understood that substituents may be attached to any carbon atom or heteroatom thereof which would otherwise bear a hydrogen atom, unless specified otherwise.

The term “Het-(C₁₋ₙ)alkyl-” as used herein and unless specified otherwise, wherein n is an integer, either alone or in combination with another radical, is intended to mean an alkyl radical having 1 to n carbon atoms as defined above which is itself substituted with a Het substituent as defined above. Examples of Het-(C₁₋ₙ)alkyl- include, but are not limited to, thienylmethyl, furylmethyl, piperidinylethyl, 2-pyridinylmethyl, 3-pyridinylmethyl, 4-pyridinylmethyl, quinolinylpropyl, and the like. When an Het-(C₁₋ₙ)alkyl- group is substituted, it is understood that substituents may be attached to either the Het or the alkyl portion thereof or both, unless specified otherwise.

The term “heteroatom” as used herein is intended to mean O, S or N.

The term “heterocycle” as used herein and unless specified otherwise, either alone or in combination with another radical, is intended to mean a 4- to 7-membered saturated, unsaturated or aromatic heterocycle containing from 1 to 4 heteroatoms each independently selected from O, N and S; or a monovalent radical derived by removal of a hydrogen atom therefrom. Examples of such heterocycles include, but are not limited to, azetidine, pyrrolidine, tetrahydrofuran, tetrahydrothiophene, thiazolidine, oxazolidine, pyrrole, thiophene, furan, pyrazole, imidazole, isoxazole, oxazole, isothiazole, thiazole, triazole, tetrazole, piperidine, piperazine, azepine, diazepine, pyran, 1,4-dioxane, 4-morpholine, 4-thiomorpholine, pyridine, pyridine-N-oxide, pyridazine, pyrazine and pyrimidine, and saturated, unsaturated and aromatic derivatives thereof.

The term “heteropolycycle” as used herein and unless specified otherwise, either alone or in combination with another radical, is intended to mean a heterocycle as defined above fused to one or more other cycle, including a carbocycle, a heterocycle or any other cycle; or a monovalent radical derived by removal of a hydrogen atom therefrom. Examples of such heteropolycycles include, but are not limited to, indole, isoindole, benzimidazole, benzothiophene, benzofuran, benzodioxole, benzothiazole, quinoline, isoquinoline, and naphthyridine.

The term “halo” as used herein is intended to mean a halogen substituent selected from fluoro, chloro, bromo or iodo.

The term “(C₁₋ₙ)haloalkyl” as used herein, wherein n is an integer, either alone or in combination with another radical, is intended to mean an alkyl radical having 1 to n carbon atoms as defined above wherein one or more hydrogen atoms are each replaced by a halo substituent. Examples of (C₁₋ₙ)haloalkyl include but are not limited to chloromethyl, chloroethyl, dichloroethyl, bromomethyl, bromoethyl, dibromoethyl, fluoromethyl, difluoromethyl, trifluoromethyl, fluoroethyl and difluoroethyl.

The terms “—O—(C₁₋ₙ)alkyl” or “(C₁₋ₙ)alkoxy” as used herein interchangeably, wherein n is an integer, either alone or in combination with another radical, is intended to mean an oxygen atom further bonded to an alkyl radical having 1 to n carbon atoms as defined above. Examples of —O—(C₁₋ₙ)alkyl include but are not limited to methoxy (CH₃O—), ethoxy (CH₃CH₂O—), propoxy (CH₃CH₂CH₂O—), 1-methylethoxy (iso-propoxy; (CH₃)₂CH—O—) and 1,1-dimethylethoxy (tert-butoxy; (CH₃)₃C—O—). When an —O—(C₁₋ₙ)alkyl radical is substituted, it is understood to be substituted on the (C₁₋ₙ)alkyl portion thereof.

The terms “—S—(C₁₋ₙ)alkyl” or “(C₁₋ₙ)alkylthio” as used herein interchangeably, wherein n is an integer, either alone or in combination with another radical, is intended to mean a sulfur atom further bonded to an alkyl radical having 1 to n carbon atoms as defined above. Examples of —S—(C₁₋ₙ)alkyl include but are not limited to methylthio (CH₃S—), ethylthio (CH₃CH₂S—), propylthio (CH₃CH₂CH₂S—), 1-methylethylthio (isopropylthio; (CH₃)₂CH—S—) and 1,1-dimethylethylthio (tert-butylthio; (CH₃)₃C—S—). When —S—(C₁₋ₙ)alkyl radical, or an oxidized derivative thereof, such as an —SO—(C₁₋ₙ)alkyl radical or an —SO₂—(C₁₋ₙ)alkyl radical, is substituted, each is understood to be substituted on the (C₁₋ₙ)alkyl portion thereof.

The term “oxo” as used herein is intended to mean an oxygen atom attached to a carbon atom as a substituent by a double bond (═O).

The term “thioxo” as used herein is intended to mean a sulfur atom attached to a carbon atom as a substituent by a double bond (═S).

The term “imino” as used herein is intended to mean a NH group attached to a carbon atom as a substituent by a double bond (═NH).

The term “cyano” or “CN” as used herein is intended to mean a nitrogen atom attached to a carbon atom by a triple bond (C≡N).

The term “COOH” as used herein is intended to mean a carboxyl group (—C(═O)—OH). It is well known to one skilled in the art that carboxyl groups may be substituted by functional group equivalents. Examples of such functional group equivalents contemplated in this invention include, but are not limited to, esters, amides, imides, boronic acids, phosphonic acids, phosphoric acids, tetrazoles, triazoles, N-acylsulfamides (RCONHSO₂NR₂), and N-acylsulfonamides (RCONHSO₂R).

The term “functional group equivalent” as used herein is intended to mean an atom or group that may replace another atom or group which has similar electronic, hybridization or bonding properties.

The following designation [symbol] is used in sub-formulas to indicate the bond which is connected to the rest of the molecule as defined.

The term “salt thereof” as used herein is intended to mean any acid and/or base addition salt of a compound according to the invention, including but not limited to a pharmaceutically acceptable salt thereof.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Berge et al., J. Pharm. Sci., 1977, 66, pp. 1-19, herein incorporated by reference.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “ester thereof” as used herein is intended to mean any ester of a compound according to the invention in which any of the —COOH substituents of the molecule is replaced by a —COOR substituent, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl, each of which being optionally further substituted.

The term “ester thereof” includes but is not limited to pharmaceutically acceptable esters thereof.

The term “pharmaceutically acceptable ester” as used herein is intended to mean esters of the compound according to the invention in which any of the COOH substituents of the molecule are replaced by a —COOR substituent, in which the R moiety of the ester is selected from alkyl (including, but not limited to, methyl, ethyl, propyl, 1-methylethyl, 1,1-dimethylethyl, butyl); alkoxyalkyl (including, but not limited to methoxymethyl); acyloxyalkyl (including, but not limited to acetoxymethyl); arylalkyl (including, but not limited to, benzyl); aryloxyalkyl (including, but not limited to, phenoxymethyl); and aryl (including, but not limited to phenyl) optionally substituted with halogen, (C₁₋₄)alkyl or (C₁₋₄)alkoxy. Other suitable esters can be found in Design of Prodrugs, Bundgaard, H. Ed. Elsevier (1985), herein incorporated by reference. Such pharmaceutically acceptable esters are usually hydrolyzed in vivo when injected into a mammal and transformed into the acid form of the compound according to the invention. With regard to the esters described above, unless otherwise specified, any alkyl moiety present preferably contains 1 to 16 carbon atoms, more preferably 1 to 6 carbon atoms. Any aryl moiety present in such esters preferably comprises a phenyl group. In particular the esters may be a (C₁₋₁₆)alkyl ester, an unsubstituted benzyl ester or a benzyl ester substituted with at least one halogen, (C₁₋₆)alkyl, (C₁₋₆)alkoxy, nitro or trifluoromethyl.

The term “mammal” as used herein is intended to encompass humans, as well as non-human mammals which are susceptible to infection by hepatitis C virus. Non-human mammals include but are not limited to domestic animals, such as cows, pigs, horses, dogs, cats, rabbits, rats and mice, and non-domestic animals.

The term “treatment” as used herein is intended to mean the administration of a compound or composition according to the present invention to alleviate or eliminate symptoms of the hepatitis C disease and/or to reduce viral load in a patient. The term “treatment” also encompasses the administration of a compound or composition according to the present invention post-exposure of the individual to the virus but before the appearance of symptoms of the disease, and/or prior to the detection of the virus in the blood, to prevent the appearance of symptoms of the disease and/or to prevent the virus from reaching detectable levels in the blood.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal.

The term “therapeutically effective amount” means an amount of a compound according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

### Preferred Embodiments

In the following preferred embodiments, groups and substituents of the compounds of formula (I):

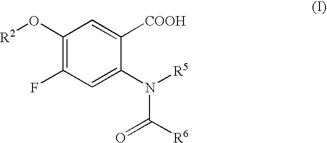

according to this invention are described in detail.

### R²:

R²-A: In one embodiment, R² is Het wherein Het is a 5- or 6-membered heterocycle containing 1 to 3 heteroatoms each independently selected from O, N and S, or a 9- or 10-membered bicyclic heteropolycycle containing 1 to 3 heteroatoms each independently selected from O, N and S; wherein Het is optionally substituted with 1 to 3 R²⁰ substituents, wherein R²⁰ is as defined herein.
R²-B: In another embodiment, R² is Het wherein Het is a 5- or 6-membered aromatic heterocycle containing 1 or 2 N heteroatoms, wherein Het is optionally substituted with 1 or 2 R²⁰ substituents, wherein R²⁰ is as defined herein.
R²-C: In another embodiment, R² is Het selected from the following formulas:

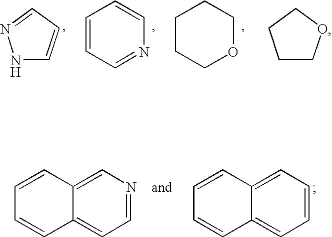

wherein Het is optionally substituted with 1 to 2 R²⁰ substituents, wherein R²⁰ is as defined herein.
R²-D: In another embodiment, R² is Het of the formula:

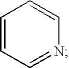

wherein Het is optionally substituted with 1 to 2 R²⁰ substituents, wherein R²⁰ is as defined herein.
R²-E: In another embodiment, R² is a group of the formula:

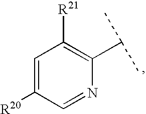

wherein R²¹ is defined as:
R²¹-A: In this embodiment, R²¹ is selected from H, halo, (C₁₋₆)alkyl, (C₁₋₆)haloalkyl and (C₃₋₇)cycloalkyl.
R²¹-B: In this embodiment, R²¹ is selected from halo, (C₁₋₆)haloalkyl and (C₃₋₇)cycloalkyl.
R²¹-C: In this embodiment, R²¹ is selected from Br, cyclopropyl, CF₃ and CHF₂.
R²¹-D: In this embodiment, R²¹ is CHF₂ or CF₃.
R²¹-E: In this embodiment, R²¹ is CF₃.
Any and each individual definition of R²¹ as set out herein may be combined with any and each individual definition of R²⁰, R⁵ and R⁶ as set out herein; and R²⁰ is as defined herein.
R²-F: In another embodiment, R² is a group of the formula:

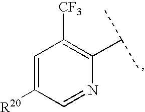

wherein R²⁰ is as defined herein.
R²-G: In another embodiment, R² is an aryl, optionally substituted with 1 to 3 R²⁰ substituents, wherein R²⁰ is as defined herein.
R²-H: In another embodiment, R² is a naphthyl or phenyl, optionally substituted with 1 or 2 R²⁰ substituents, wherein R²⁰ is as defined herein.
R²-I: In another embodiment, R² is a group of the formula:

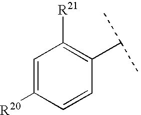

wherein R²¹ and R²⁰ are as defined herein.
R²-J: In another embodiment, R² is a group of the formula:

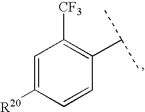

wherein R²⁰ is as defined herein.
R²-K: In another embodiment, R² is selected from the following group of formulas:

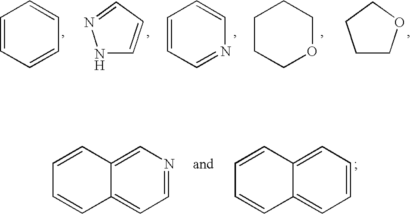

wherein R² is optionally substituted with 1 or 2 R²⁰ substituents, wherein R²⁰ is as defined herein.
R²-L: In another embodiment, R² is selected from the following group of formulas:

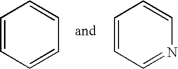

wherein R² is optionally substituted with 1 or 2 R²⁰ substituents, wherein R²⁰ is as defined herein.
R²-M: In another embodiment, R² is selected from the group of formulas:

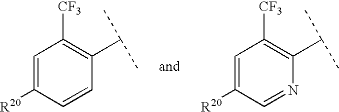

wherein R²⁰ is as defined herein.
R²-N: In another embodiment, R² is aryl or Het, optionally substituted with 1 to 5 R²⁰ substituents wherein R²⁰ is as defined herein.

Any and each individual definition of R² as set out herein may be combined with any and each individual definition of R²⁰, R⁵ and R⁶ as set out herein.
R²⁰-A: In one embodiment, R²⁰ is selected from:
   b) R⁷, wherein R⁷ is selected from H, (C₁₋₆)alkyl, (C₁₋₆)haloalkyl, (C₃₋₇)cycloalkyl and Het;
      wherein the Het is optionally substituted with 1 to 3 substituents each independently selected from:
      i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
      ii) (C₁₋₆)alkyl optionally substituted with —OH, or —O—(C₁₋₆)alkyl; and
      iii) aryl or Het, wherein each of the aryl and Het is optionally substituted with halo or (C₁₋₆)alkyl;
   c) —C(═O)—R⁷, —C(═O)—O—R⁷, —O—R⁷, —S—R⁷, —SO—R⁷, —SO₂—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is as defined above;
   d) aryl-(C₁₋₆)alkyl or Het-(C₁₋₆)alkyl,
      wherein each of the aryl and Het is optionally substituted with 1 to 3 substituents each independently selected from:
      i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
      ii) (C₁₋₆)alkyl optionally substituted with —OH or —O—(C₁₋₆)alkyl; and
      iii) aryl or Het, wherein each of the aryl and Het is optionally substituted with halo or (C₁₋₆)alkyl; and
   e) —N(R⁸)R⁹, —C(═O)—N(R⁸)R⁹, —SO₂—N(R⁸)R⁹, or —(C₁₋₆)alkylene-N(R⁸)R⁹, wherein
      R⁸ is in each instance independently selected from H and (C₁₋₆)alkyl; and
      R⁹ is in each instance independently selected from R⁷, —(C₃₋₇)cycloalkyl-(C₁₋₆)alkyl, —C(═O)—R¹⁰, —C(═O)OR¹⁰ and —C(═O)N(H)R¹⁰;
      wherein R⁷ is as defined above;
      wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
      wherein R¹⁹ is in each instance independently selected from (C₁₋₆)alkyl and Het, wherein said Het is optionally substituted with (C₁₋₆)alkyl; and
      wherein the (C₁₋₆)alkyl is optionally substituted with 1 or 2 substituents each independently selected from COOH, —NH₂, —NH(C₁₋₄)alkyl, and —N((C₁₋₄)alkyl)₂.
R²⁰-B: In one embodiment, R²⁰ is selected from:
   c) —C(═O)—Het, —(C₁₋₆)alkylene-O—Het, —(C₁₋₆)alkylene-S-Het;
      wherein the Het is optionally substituted with 1 to 2 substituents each independently selected from: (C₁₋₆)alkyl;
   d) Het-(C₁₋₆)alkyl,
      wherein the Het is optionally substituted with 1 to 2 substituents each independently selected from:
      i) halo, —OH, —NH₂, —NH(C₁₋₄)alkyl, —N((C₁₋₄)alkyl)₂, or —NH—C(═O)(C₁₋₄)alkyl; and
      ii) (C₁₋₆)alkyl —O—(C₁₋₆)alkyl; and
   e) —(C₁₋₆)alkylene-N(H)R⁹, wherein
      R⁹ is in each instance independently selected from Het, being optionally substituted with 1 or 2 substituents each independently selected from (C₁₋₆)alkyl, halo, O—(C₁₋₆)alkyl, —NH₂, —NH(C₁₋₄)alkyl, and —N((C₁₋₄)alkyl)₂.
R²⁰-C: In one embodiment, R²⁰ is selected from:
   c) —(C₁₋₆)alkylene-O—Het, —(C₁₋₆)alkylene-S-Het;
      wherein the Het is optionally substituted with 1 to 2 substituents each independently selected from (C₁₋₆)alkyl; and
      wherein Het is defined as:

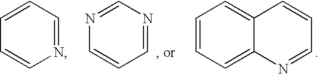

d) Het-(C₁₋₆)alkyl,
   wherein the Het is optionally substituted with 1 to 2 substituents each independently selected from:
   i) halo, —OH, —NH₂, —NH(C₁₋₄)alkyl, —N((C₁₋₄)alkyl)₂, or —NH—C(═O)(C₁₋₄)alkyl; and
   ii) (C₁₋₆)alkyl; and
   wherein Het is defined as:

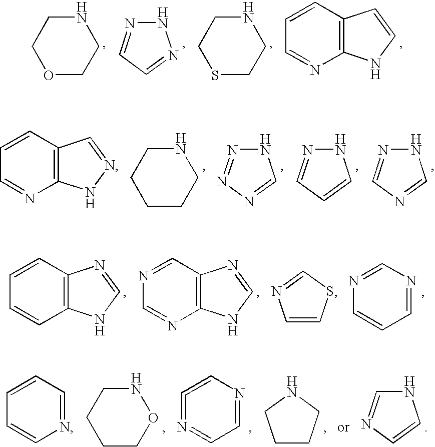

e) —(C₁₋₆)alkylene-N(H)R⁹, wherein
   R⁹ is in each instance independently selected from Het, being optionally substituted with 1 or 2 substituents each independently selected from (C₁₋₆)alkyl, halo, O—(C₁₋₆)alkyl, —NH₂, —NH(C₁₋₄)alkyl, and —N((C₁₋₄)alkyl)₂;
   and wherein Het is defined as:

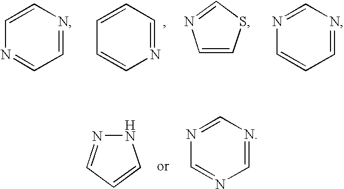

R²⁰-D: In one embodiment, R²⁰ is selected from:

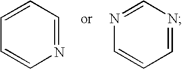

b) Het, wherein Het is defined as
c) —(C₁₋₆)alkylene-O—Het, —(C₁₋₆)alkylene-S-Het;
   wherein the Het is optionally substituted with 1 to 2 substituents each independently selected from (C₁₋₆)alkyl and (C₁₋₆)haloalkyl; and
   wherein Het is defined as:

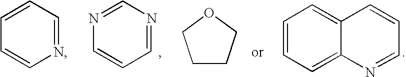

d) Het-(C₁₋₆)alkyl,
   wherein the Het is optionally substituted with 1 to 2 substituents each independently selected from:
   i) halo, —OH, —NH₂, —NH(C₁₋₄)alkyl, —N((C₁₋₄)alkyl)₂, or —NH—C(═O)(C₁₋₄)alkyl; and
   ii) (C₁₋₆)alkyl —O—(C₁₋₆)alkyl; and
   wherein Het is defined as:

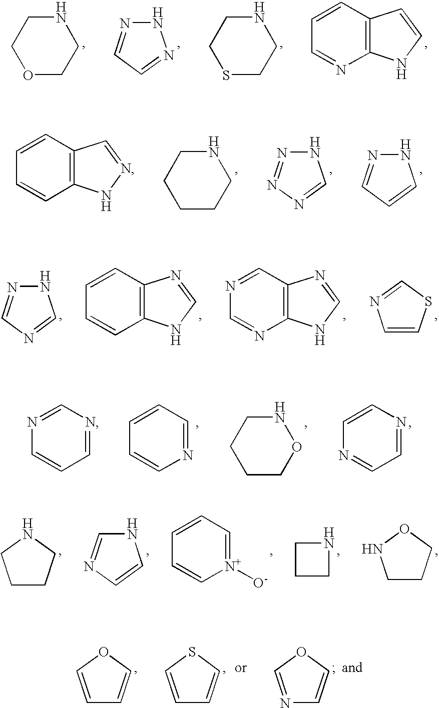

e) —(C₁₋₆)alkylene-N(H)R⁹, wherein
   R⁹ is in each instance independently selected from Het, being optionally substituted with 1 or 2 substituents each independently selected from (C₁₋₆)alkyl, halo, O—(C₁₋₆)alkyl, —NH₂, —NH(C₁₋₄)alkyl, and —N((C₁₋₄)alkyl)₂;
   and wherein Het is defined as:

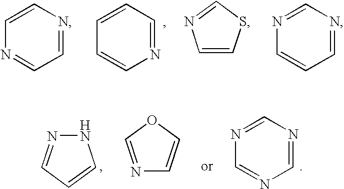

R²⁰-E: In one embodiment, R²⁰ is selected from:
   b) R⁷, wherein R⁷ is as defined as Het; wherein the Het is optionally substituted with 1 to 3 substituents each independently selected from:
      i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
      ii) (C₁₋₆)alkyl optionally substituted with —OH, or —O—(C₁₋₆)alkyl; and
      iii) Het
   c) —C(═O)—R⁷, —(C₁₋₆)alkylene-O—R⁷, —(C₁₋₆)alkylene-S—R⁷,
      wherein R⁷ is as defined above;
   d) Het-(C₁₋₆)alkyl,
      wherein each of the aryl and Het is optionally substituted with 1 to 3 substituents each independently selected from:
      i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
      ii) (C₁₋₆)alkyl optionally substituted with —OH or —O—(C₁₋₆)alkyl; and
      iii) aryl or Het, wherein each of the aryl and Het is optionally substituted with halo or (C₁₋₆)alkyl; and
   e) —(C₁₋₆)alkylene-N(R⁸)R⁹, wherein
      R⁸ is in each instance independently selected from H and (C₁₋₆)alkyl; and
      R⁹ is in each instance independently selected from R⁷, —(C₃₋₇)cycloalkyl-(C₁₋₆)alkyl, —C(═O)—R¹⁰, —C(═O)OR¹⁰ and —C(═O)N(H)R¹⁰;
      wherein R⁷ is as defined above;
      wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
      wherein R¹⁰ is in each instance independently selected from (C₁₋₆)alkyl and Het, wherein said Het is optionally substituted with (C₁₋₆)alkyl; and
R²⁰-F: In one embodiment, R²⁰ is selected from:
   c) —(C₁₋₆)alkylene-O—Het, —(C₁₋₆)alkylene-S-Het;
      wherein the Het is optionally substituted with 1 to 2 substituents each independently selected from —CH₃; and
      wherein Het is defined as:

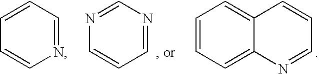

d) Het-(C₁₋₆)alkyl,
   wherein the Het is optionally substituted with 1 to 2 substituents each independently selected from:
   i) F, —NH₂, —NH(CH₂CH₃), —N(CH₃)₂; and
   ii) —CH₃, CH₂CH(CH₃)₂ and
   wherein Het is defined as:

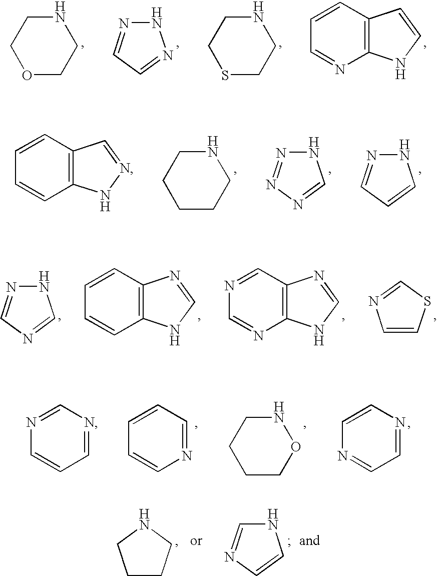

e) —(C₁₋₆)alkylene-N(H)R⁹, wherein
   R⁹ is in each instance independently selected from Het, being optionally substituted with 1 or 2 substituents each independently selected from —CH₃, Cl, Br and OCH₃;
   and wherein Het is defined as:

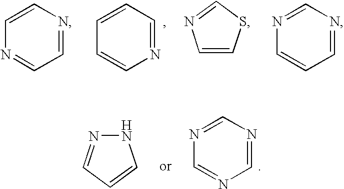

R²⁰-G: In still another embodiment, R²⁰ is selected from:
   c) —(C₁₋₆)alkylene-O—R⁷, —(C₁₋₆)alkylene-S—R⁷, wherein R⁷ is defined as:

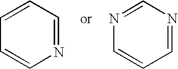

wherein R⁷ is optionally substituted with 1 to 2 substituents each independently selected from (C₁₋₆)alkyl;
d) Het-(C₁₋₆)alkyl,
   wherein the Het is selected from:

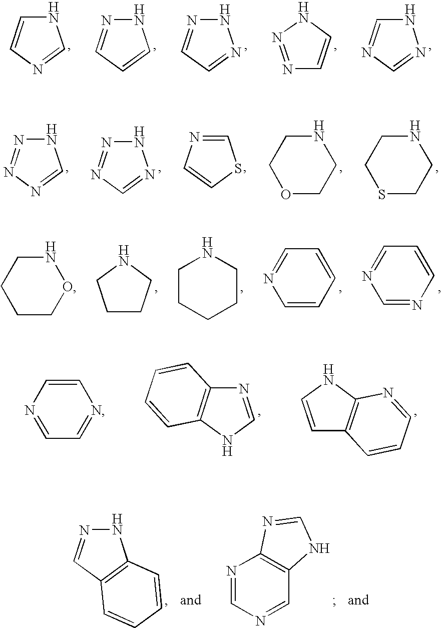

wherein the Het is optionally substituted with 1 to 2 substituents each independently selected from: halo, —NH₂, —NH(C₁₋₄)alkyl, and —N((C₁₋₄)alkyl)₂, (C₁₋₆)alkyl; and
e) —(C₁₋₆)alkylene-N(R⁹)R⁹, wherein
   R⁸ is in each instance H; and
   R⁹ is in each instance independently selected from:

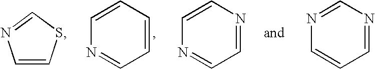

wherein R⁹ is optionally substituted with 1 or 2 substituents each independently selected from halo and (C₁₋₄)alkyl.
R²⁰-H: In still another embodiment, R²⁰ is selected from the group of formulas:

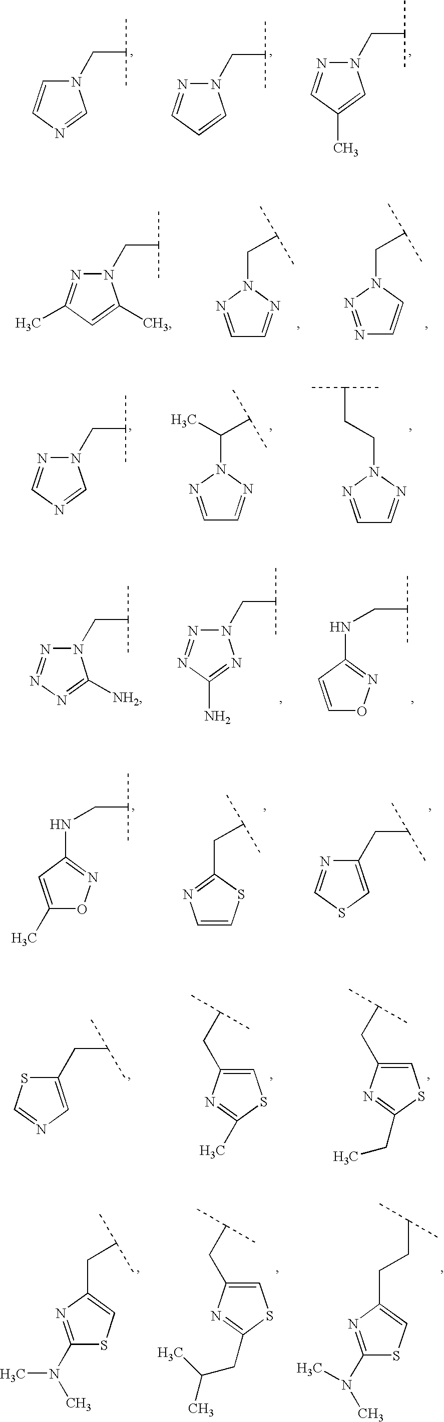

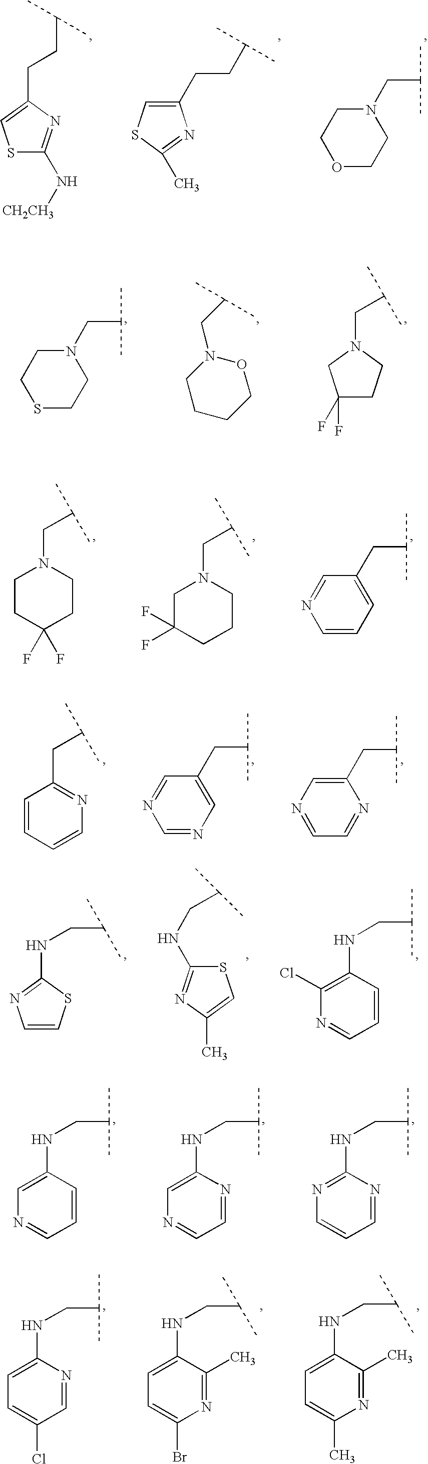

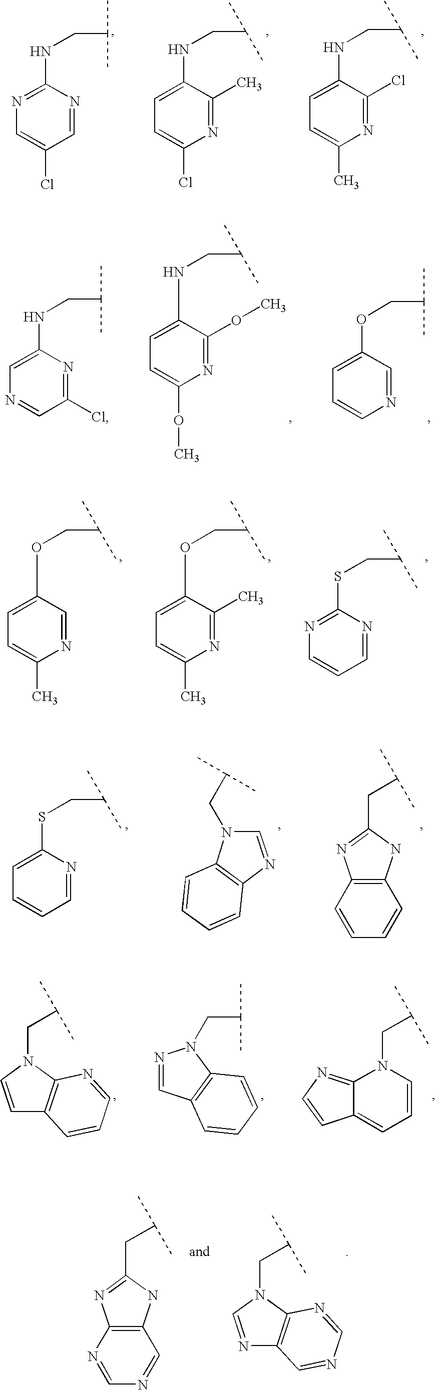

R²⁰-I: In one embodiment, R²⁰ is selected from:
   a) halo;
   b) R⁷, wherein R⁷ is selected from H, (C₁₋₆)alkyl, (C₁₋₆)haloalkyl, (C₃₋₇)cycloalkyl, aryl and Het;
      wherein each of the aryl and Het is optionally substituted with 1 to 3 substituents each independently selected from:
      i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
      ii) (C₁₋₆)alkyl optionally substituted with —OH, or —O—(C₁₋₆)alkyl; and
      iii) aryl or Het, wherein each of the aryl and Het is optionally substituted with halo or (C₁₋₆)alkyl;
   c) —C(═O)—R⁷, —C(═O)—O—R⁷, —O—R⁷, —S—R⁷, —SO—R⁷, —SO₂—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is as defined above;
   d) aryl-(C₁₋₆)alkyl or Het-(C₁₋₆)alkyl,
      wherein each of the aryl and Het is optionally substituted with 1 to 3 substituents each independently selected from:
      i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
      ii) (C₁₋₆)alkyl optionally substituted with —OH or —O—(C₁₋₆)alkyl; and
      iii) aryl or Het, wherein each of the aryl and Het is optionally substituted with halo or (C₁₋₆)alkyl; and
   e) —N(R⁹)R⁹, —C(═O)—N(R⁹)R⁹, —SO₂—N(R⁹)R⁹, or —(C₁₋₆)alkylene-N(R⁹)R⁹, wherein
      R⁸ is in each instance independently selected from H and (C₁₋₆)alkyl; and
      R⁹ is in each instance independently selected from R⁷, —(C₃₋₇)cycloalkyl-(C₁₋₆)alkyl, —C(═O)—R¹⁰, —C(═O)OR¹⁹ and —C(═O)N(H)R¹⁰;
      wherein R⁷ is as defined above;
      wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
      wherein R¹⁰ is in each instance independently selected from (C₁₋₆)alkyl, and Het, wherein said Het is optionally substituted with (C₁₋₆)alkyl; and
      wherein the (C₁₋₆)alkyl is optionally substituted with 1 or 2 substituents each independently selected from COOH, —NH₂, —NH(C₁₋₄)alkyl, and —N((C₁₋₄)alkyl)₂.
R²⁰-J: In one embodiment, R²⁰ is selected from:
   a) halo;
   b) R⁷, wherein R⁷ is selected from H, (C₁₋₆)alkyl, (C₁₋₆)haloalkyl, (C₃₋₇)cycloalkyl, aryl and Het;
      wherein the (C₁₋₆)alkyl and (C₃₋₇)cycloalkyl are optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂
      wherein each of the aryl and Het is optionally substituted with 1 to 3 substituents each independently selected from:
      i) halo, cyano, oxo, thioxo, imino, —OH, —O—(C₁₋₆)alkyl, —O—(C₁₋₆)haloalkyl, O—(C₃₋₇)cycloalkyl, (C₃₋₇)cycloalkyl, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl or Het, wherein each of the aryl and Het is optionally substituted with halo, (C₁₋₆)alkyl or —O—(C₁₋₆)alkyl;
   c) —C(═O)—R⁷, —C(═O)—O—R⁷, —O—R⁷, —S—R⁷, —SO—R⁷, —SO₂—R⁷, —(C₁₋₆)alkylene-R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
      wherein R⁷ is as defined above; and wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
   d) aryl-(C₁₋₆)alkyl or Het-(C₁₋₆)alkyl,
      wherein each of the aryl and Het is optionally substituted with 1 to 3 substituents each independently selected from:
      i) halo, cyano, oxo, thioxo, imino, —OH, —O—(C₁₋₆)alkyl, —O—(C₁₋₆)haloalkyl, O—(C₃₋₇)cycloalkyl, (C₃₋₇)cycloalkyl, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
      ii) (C₁₋₆)alkyl optionally substituted with —OH, —O—(C₁₋₆)haloalkyl, or —O—(C₁₋₆)alkyl; and
      iii) aryl or Het, wherein each of the aryl and Het is optionally substituted with halo, (C₁₋₆)alkyl or —O—(C₁₋₆)alkyl;
      wherein the —(C₁₋₆)alkyl portion of the aryl-(C₁₋₆)alkyl or Het-(C₁₋₆)alkyl is optionally substituted with 1 or 2 substituents each independently selected from —OH, —(C₁₋₆)alkyl, halo, —(C₁₋₆)haloalkyl, (C₃₋₇)cycloalkyl, O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂; and
   e) —N(R⁸)R⁹, —C(═O)—N(R⁸)R⁹, —SO₂—N(R⁸)R⁹, or —(C₁₋₆)alkylene-N(R⁸)R⁹ wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
      R⁸ is in each instance independently selected from H, (C₁₋₆)alkyl and (C₃₋₇)cycloalkyl; and
      R⁹ is in each instance independently selected from R⁷, —O— (C₁₋₆)alkyl, —(C₁₋₆)alkylene-R⁷, —(C₃₋₇)cycloalkyl-(C₁₋₆)alkyl, —C(═O)—R¹⁰, —C(═O)OR¹⁰ and —C(═O)N(H)R¹⁰;
      wherein R⁷ is as defined above;
      wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂
      wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
      wherein R¹⁰ is in each instance independently selected from (C₁₋₆)alkyl, and Het, wherein said Het is optionally substituted with (C₁₋₆)alkyl;
         or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
            wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl.

Any and each individual definition of R²⁰ as set out herein may be combined with any and each individual definition of R², R⁵ and R⁶ as set out herein.

### R⁵:

R⁵-A: In one embodiment, R⁵ is H or (C₁₋₆)alkyl, wherein the (C₁₋₆)alkyl is optionally substituted with 1 to 4 substituents each independently selected from —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂, and —SO₂(C₁₋₆)alkyl.
R⁵-B: In another embodiment, R⁵ is selected from (C₁₋₄)alkyl, wherein the (C₁₋₄)alkyl is optionally substituted with 1 or 2 substituents each independently selected from —OH and —COOH.
R⁵-C: In still another embodiment, R⁵ is selected from methyl, ethyl, propyl, 1-methylethyl,

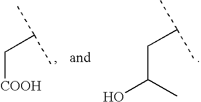

R⁵-D: In yet another embodiment, R⁵ is methyl, ethyl, propyl or 1-methylethyl.
R⁵-E: In a further embodiment, R⁵ is 1-methylethyl.
R⁵-F: In an alternative embodiment, R⁵ is Het optionally substituted with 1 to 4 substituents each independently selected from (C₁₋₆)alkyl, —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂, and —SO₂(C₁₋₆)alkyl.
R⁵-G: In another alternative embodiment, R⁵ is a 5- or 6-membered saturated heterocycle containing 1 to 3 heteroatoms each independently selected from O, N and S, the heterocycle being optionally substituted with 1 to 4 substituents each independently selected from (C₁₋₄)alkyl, —C(═O)—(C₁₋₄)alkyl, —C(═O)—O—(C₁₋₄)alkyl, —C(═O)—NH—(C₁₋₄)alkyl, —C(═O)—N((C₁₋₄)alkyl)₂, and —SO₂(C₁₋₄)alkyl.
R⁵-H: In yet another alternative embodiment, R⁵ is a 6-membered saturated heterocycle containing 1 or 2 heteroatoms each independently selected from O and N, the heterocycle being optionally substituted with 1 or 2 substituents each independently selected from CH₃, —C(═O)—CH₃, —C(═O)—O—CH₃, —C(═O)—O—C(CH₃)₃, —C(═O)—NH—CH₂CH₃ and —SO₂CH₃.
R⁵-I: In yet another alternative embodiment, R⁵ is

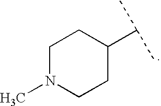

R⁵-J: In yet another alternative embodiment, R⁵ is (C₁₋₆)alkyl or (C₃₋₇)cycloalkyl.
R⁵-K: In yet another alternative embodiment, R⁵ is 1-methylethyl or cyclobutyl.
R⁵-L: In still another alternative embodiment, R⁵ is 1-methylethyl, cyclobutyl or

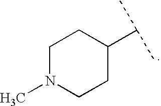

R⁵-M: In still another alternative embodiment, R⁵ is selected from H, (C₁₋₆)alkyl, (C₃₋₇)cycloalkyl, and Het; the (C₁₋₆)alkyl and Het each being optionally substituted with 1 to 4 substituents each independently selected from (C₁₋₆)alkyl, —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂, and —SO₂(C₁₋₆)alkyl.
R⁵-N: In still another alternative embodiment, R⁵ is selected from H, (C₁₋₆)alkyl, (C₃₋₇)cycloalkyl, (C₃₋₇)cycloalkyl-(C₁₋₆)alkyl and Het; the (C₁₋₆)alkyl and Het each being optionally substituted with 1 to 4 substituents each independently selected from (C₁₋₆)alkyl, —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂, and —SO₂(C₁₋₆)alkyl.

Any and each individual definition of R⁵ as set out herein may be combined with any and each individual definition of R², R²⁰ and R⁶ as set out herein.

### R⁶:

R⁶-A: In one embodiment, R⁶ is selected from (C₅₋₇)cycloalkyl, the (C₅₋₇)cycloalkyl being optionally substituted with 1 to 5 substituents each independently selected from halo, (C₁₋₆)alkyl, (C₁₋₆)haloalkyl, —OH, —SH, —O—(C₁₋₄)alkyl and —S—(C₁₋₄)alkyl.
R⁶-B: In another embodiment, R⁶ is cyclopentyl, cyclohexyl or cycloheptyl, the cyclopentyl, cyclohexyl and cycloheptyl each being optionally substituted with 1 to 3 substituents each independently selected from halo, —OH, (C₁₋₄)alkyl and (C₁₋₄)haloalkyl.
R⁶-C: In yet another embodiment, R⁶ is cyclohexyl optionally substituted with 1 to 3 substituents each independently selected from fluoro, (C₁₋₄)alkyl and (C₁₋₄haloalkyl.
R⁶-D: In still another embodiment, R⁶ is selected from:

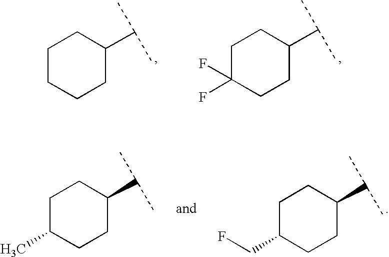

R⁶-E: In still another embodiment, R⁶ is

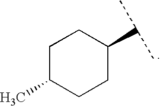

R⁶-F: In an alternative embodiment, R⁶ is aryl optionally substituted with 1 to 5 substituents each independently selected from halo, (C₁₋₆)alkyl, (C₁₋₆)haloalkyl, —OH, —SH, —O—(C₁₋₄)alkyl and —S—(C₁₋₄)alkyl.
R⁶-G: In another alternative embodiment, R⁶ is phenyl optionally substituted with 1 to 3 substituents each independently selected from halo, (C₁₋₄)alkyl, —OH, (C₁₋₄)haloalkyl and —O—(C₁₋₄)alkyl.
R⁶-H: In yet another alternative embodiment, R⁶ is phenyl optionally substituted with 1 to 3 substituents each independently selected from F, Cl, Br, —OH and —O—CH₃.
R⁶-I: In vet another embodiment. R⁶ is selected from:

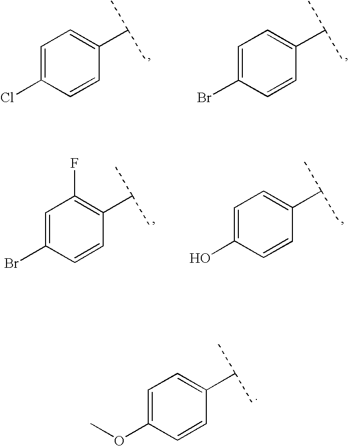

R⁶-J: In yet another embodiment, R⁶ is selected from:

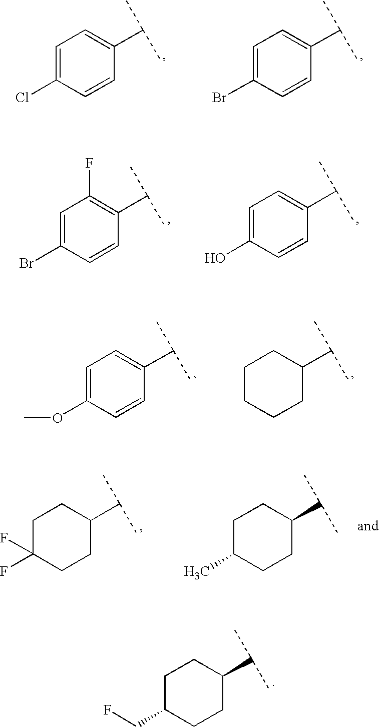

R⁶-K: In yet another embodiment, R⁶ is selected from (C₅₋₇)cycloalkyl and aryl; the (C₅₋₇)cycloalkyl and aryl each being optionally substituted with 1 to 5 substituents each independently selected from halo, (C₁₋₆)alkyl, (C₁₋₆)haloalkyl, —OH, —SH, —O—(C₁₋₄)alkyl and —S—(C₁₋₄)alkyl.
R⁶-L: In yet another embodiment, R⁶ is selected from (C₃₋₇)cycloalkyl and aryl; the (C₃₋₇)cycloalkyl and aryl each being optionally substituted with 1 to 5 substituents each independently selected from halo, (C₁₋₆)alkyl, (C₁₋₆)haloalkyl, (C₃₋₇)cycloalkyl, —OH, —SH, —O—(C₁₋₄)alkyl and —S—(C₁₋₄)alkyl.

Any and each individual definition of R⁶ as set out herein may be combined with any and each individual definition of R², R²⁰ and R⁵ as set out herein.

Examples of preferred subgeneric embodiments of the present invention are set forth in the following table, wherein each substituent group of each embodiment is defined according to the definitions set forth above:

| Embodiment | R² | R²⁰ | R⁵ | R⁶ |
|---|---|---|---|---|
| E-1 | R²-A | R²⁰-A | R⁵-B | R⁶-J |
| E-2 | R²-A | R²⁰-C | R⁵-E | R⁶-E |
| E-3 | R²-A | R²⁰-A | R⁵-J | R⁶-J |
| E-4 | R²-A | R²⁰-C | R⁵-K | R⁶-E |
| E-5 | R²-A | R²⁰-A | R⁵-B | R⁶-J |
| E-6 | R²-A | R²⁰-C | R⁵-E | R⁶-E |
| E-7 | R²-A | R²⁰-A | R⁵-J | R⁶-J |
| E-8 | R²-B | R²⁰-A | R⁵-B | R⁶-E |
| E-9 | R²-B | R²⁰-C | R⁵-D | R⁶-J |
| E-10 | R²-B | R²⁰-D | R⁵-E | R⁶-J |
| E-11 | R²-B | R²⁰-F | R⁵-J | R⁶-E |
| E-12 | R²-B | R²⁰-G | R⁵-E | R⁶-C |
| E-13 | R²-B | R²⁰-A | R⁵-E | R⁶-J |
| E-14 | R²-C | R²⁰-C | R⁵-E | R⁶-J |
| E-15 | R²-C | R²⁰-E | R⁵-J | R⁶-E |
| E-16 | R²-C | R²⁰-F | R⁵-K | R⁶-A |
| E-17 | R²-C | R²⁰-G | R⁵-L | R⁶-D |
| E-18 | R²-C | R²⁰-H | R⁵-A | R⁶-J |
| E-19 | R²-C | R²⁰-B | R⁵-B | R⁶-E |
| E-20 | R²-C | R²⁰-A | R⁵-F | R⁶-E |
| E-21 | R²-C | R²⁰-F | R⁵-J | R⁶-D |
| E-22 | R²-C | R²⁰-E | R⁵-E | R⁶-J |
| E-23 | R²-C | R²⁰-B | R⁵-J | R⁶-E |
| E-24 | R²-D | R²⁰-G | R⁵-E | R⁶-E |
| E-25 | R²-K | R²⁰-C | R⁵-E | R⁶-D |
| E-26 | R²-K | R²⁰-C | R⁵-J | R⁶-E |
| E-27 | R²-K | R²⁰-C | R⁵-J | R⁶-J |
| E-28 | R²-K | R²⁰-D | R⁵-E | R⁶-E |
| E-29 | R²-K | R²⁰-D | R⁵-J | R⁶-J |
| E-30 | R²-K | R²⁰-D | R⁵-K | R⁶-D |
| E-31 | R²-K | R²⁰-D | R⁵-K | R⁶-J |
| E-32 | R²-K | R²⁰-E | R⁵-E | R⁶-E |
| E-33 | R²-K | R²⁰-E | R⁵-E | R⁶-J |
| E-34 | R²-K | R²⁰-F | R⁵-E | R⁶-D |
| E-35 | R²-K | R²⁰-F | R⁵-E | R⁶-E |
| E-36 | R²-K | R²⁰-F | R⁵-K | R⁶-J |
| E-37 | R²-K | R²⁰-F | R⁵-L | R⁶-D |
| E-38 | R²-K | R²⁰-G | R⁵-E | R⁶-D |
| E-39 | R²-K | R²⁰-G | R⁵-E | R⁶-E |
| E-40 | R²-K | R²⁰-G | R⁵-J | R⁶-J |
| E-41 | R²-K | R²⁰-G | R⁵-J | R⁶-D |
| E-42 | R²-K | R²⁰-G | R⁵-K | R⁶-D |
| E-43 | R²-K | R²⁰-H | R⁵-L | R⁶-D |
| E-44 | R²-K | R²⁰-H | R⁵-L | R⁶-D |
| E-45 | R²-K | R²⁰-H | R⁵-E | R⁶-B |
| E-46 | R²-K | R²⁰-H | R⁵-B | R⁶-D |
| E-47 | R²-K | R²⁰-H | R⁵-K | R⁶-E |
| E-48 | R²-K | R²⁰-H | R⁵-L | R⁶-J |
| E-49 | R²-K | R²⁰-H | R⁵-E | R⁶-E |
| E-50 | R²-L | R²⁰-C | R⁵-E | R⁶-D |
| E-51 | R²-L | R²⁰-C | R⁵-J | R⁶-E |
| E-52 | R²-L | R²⁰-C | R⁵-J | R⁶-J |
| E-53 | R²-L | R²⁰-D | R⁵-E | R⁶-E |
| E-54 | R²-L | R²⁰-D | R⁵-J | R⁶-J |
| E-55 | R²-L | R²⁰-D | R⁵-K | R⁶-D |
| E-56 | R²-L | R²⁰-D | R⁵-K | R⁶-J |
| E-57 | R²-L | R²⁰-E | R⁵-E | R⁶-E |
| E-58 | R²-L | R²⁰-E | R⁵-E | R⁶-J |
| E-59 | R²-L | R²⁰-F | R⁵-E | R⁶-D |
| E-60 | R²-L | R²⁰-F | R⁵-E | R⁶-E |
| E-61 | R²-L | R²⁰-F | R⁵-K | R⁶-J |
| E-62 | R²-L | R²⁰-F | R⁵-L | R⁶-D |
| E-63 | R²-L | R²⁰-G | R⁵-E | R⁶-D |
| E-64 | R²-L | R²⁰-G | R⁵-E | R⁶-E |
| E-65 | R²-L | R²⁰-G | R⁵-J | R⁶-J |
| E-66 | R²-L | R²⁰-G | R⁵-J | R⁶-D |
| E-67 | R²-L | R²⁰-G | R⁵-K | R⁶-D |
| E-68 | R²-L | R²⁰-H | R⁵-L | R⁶-D |
| E-69 | R²-L | R²⁰-H | R⁵-L | R⁶-D |
| E-70 | R²-L | R²⁰-H | R⁵-E | R⁶-B |
| E-71 | R²-L | R²⁰-H | R⁵-B | R⁶-D |
| E-72 | R²-L | R²⁰-H | R⁵-K | R⁶-E |
| E-73 | R²-L | R²⁰-H | R⁵-L | R⁶-J |
| E-74 | R²-L | R²⁰-H | R⁵-E | R⁶-E |
| E-75 | R²-M | R²⁰-C | R⁵-E | R⁶-D |
| E-76 | R²-M | R²⁰-C | R⁵-J | R⁶-E |
| E-77 | R²-M | R²⁰-C | R⁵-J | R⁶-J |
| E-78 | R²-M | R²⁰-D | R⁵-E | R⁶-E |
| E-79 | R²-M | R²⁰-D | R⁵-J | R⁶-J |
| E-80 | R²-M | R²⁰-D | R⁵-K | R⁶-D |
| E-81 | R²-M | R²⁰-D | R⁵-K | R⁶-J |
| E-82 | R²-M | R²⁰-E | R⁵-E | R⁶-E |
| E-83 | R²-M | R²⁰-E | R⁵-E | R⁶-J |
| E-84 | R²-M | R²⁰-F | R⁵-E | R⁶-D |
| E-85 | R²-M | R²⁰-F | R⁵-E | R⁶-E |
| E-86 | R²-M | R²⁰-F | R⁵-K | R⁶-J |
| E-87 | R²-M | R²⁰-F | R⁵-L | R⁶-D |
| E-88 | R²-M | R²⁰-G | R⁵-E | R⁶-D |
| E-89 | R²-M | R²⁰-G | R⁵-E | R⁶-E |
| E-90 | R²-M | R²⁰-G | R⁵-J | R⁶-J |
| E-91 | R²-M | R²⁰-G | R⁵-J | R⁶-D |
| E-92 | R²-M | R²⁰-G | R⁵-K | R⁶-D |
| E-93 | R²-M | R²⁰-H | R⁵-L | R⁶-D |
| E-94 | R²-M | R²⁰-H | R⁵-L | R⁶-D |
| E-95 | R²-M | R²⁰-H | R⁵-E | R⁶-B |
| E-96 | R²-M | R²⁰-H | R⁵-BJ | R⁶-D |
| E-97 | R²-M | R²⁰-H | R⁵-K | R⁶-E |
| E-98 | R²-M | R²⁰-H | R⁵-L | R⁶-J |
| E-99 | R²-M | R²⁰-H | R⁵-E | R⁶-E |
| E-100 | R²-D | R²⁰-C | R⁵-K | R⁶-E |
| E-101 | R²-D | R²⁰-A | R⁵-B | R⁶-J |
| E-102 | R²-D | R²⁰-C | R⁵-E | R⁶-E |
| E-103 | R²-D | R²⁰-A | R⁵-J | R⁶-J |
| E-104 | R²-J | R²⁰-A | R⁵-B | R⁶-E |
| E-105 | R²-J | R²⁰-C | R⁵-D | R⁶-J |
| E-106 | R²-J | R²⁰-D | R⁵-E | R⁶-J |
| E-107 | R²-J | R²⁰-F | R⁵-J | R⁶-E |
| E-108 | R²-J | R²⁰-G | R⁵-E | R⁶-C |
| E-109 | R²-J | R²⁰-A | R⁵-E | R⁶-J |
| E-110 | R²-F | R²⁰-C | R⁵-E | R⁶-J |
| E-111 | R²-F | R²⁰-E | R⁵-J | R⁶-E |
| E-112 | R²-F | R²⁰-F | R⁵-K | R⁶-A |
| E-113 | R²-F | R²⁰-G | R⁵-L | R⁶-D |
| E-114 | R²-F | R²⁰-H | R⁵-A | R⁶-J |
| E-115 | R²-F | R²⁰-B | R⁵-B | R⁶-E |
| E-116 | R²-F | R²⁰-A | R⁵-F | R⁶-E |
| E-117 | R²-F | R²⁰-F | R⁵-J | R⁶-D |
| E-118 | R²-F | R²⁰-E | R⁵-E | R⁶-J |
| E-119 | R²-H | R²⁰-C | R⁵-E | R⁶-J |
| E-120 | R²-H | R²⁰-E | R⁵-J | R⁶-E |
| E-121 | R²-H | R²⁰-F | R⁵-K | R⁶-A |
| E-122 | R²-H | R²⁰-G | R⁵-L | R⁶-D |
| E-123 | R²-H | R²⁰-H | R⁵-A | R⁶-J |
| E-124 | R²-H | R²⁰-B | R⁵-B | R⁶-E |
| E-125 | R²-H | R²⁰-A | R⁵-F | R⁶-E |
| E-126 | R²-H | R²⁰-F | R⁵-J | R⁶-D |
| E-127 | R²-H | R²⁰-E | R⁵-E | R⁶-J |
| E-128 | R²-M | R²⁰-A | R⁵-A | R⁶-A |
| E-129 | R²-M | R²⁰-B | R⁵-B | R⁶-B |
| E-130 | R²-M | R²⁰-B | R⁵-C | R⁶-C |
| E-131 | R²-M | R²⁰-C | R⁵-D | R⁶-F |
| E-132 | R²-M | R²⁰-C | R⁵-F | R⁶-G |
| E-133 | R²-M | R²⁰-D | R⁵-G | R⁶-B |
| E-134 | R²-M | R²⁰-D | R⁵-H | R⁶-A |
| E-135 | R²-M | R²⁰-E | R⁵-I | R⁶-C |
| E-136 | R²-M | R²⁰-E | R⁵-J | R⁶-G |
| E-137 | R²-M | R²⁰-F | R⁵-E | R⁶-H |
| E-138 | R²-E | R²⁰-G | R⁵-C | R⁶-I |
| E-139 | R²-E | R²⁰-E | R⁵-E | R⁶-J |
| E-140 | R²-E | R²⁰-A | R⁵-A | R⁶-A |
| E-141 | R²-E | R²⁰-B | R⁵-B | R⁶-B |
| E-142 | R²-E | R²⁰-B | R⁵-C | R⁶-C |
| E-143 | R²-G | R²⁰-B | R⁵-B | R⁶-E |
| E-144 | R²-G | R²⁰-A | R⁵-F | R⁶-E |
| E-145 | R²-G | R²⁰-F | R⁵-J | R⁶-D |
| E-146 | R²-G | R²⁰-E | R⁵-E | R⁶-J |
| E-147 | R²-G | R²⁰-A | R⁵-A | R⁶-A |
| E-148 | R²-G | R²⁰-G | R⁵-M | R⁶-I |
| E-149 | R²-G | R²⁰-B | R⁵-L | R⁶-F |
| E-150 | R²-G | R²⁰-C | R⁵-J | R⁶-F |

A further preferred embodiment of the present invention the compound according to formula (I) wherein:
R² is aryl or Het, optionally substituted with R²⁰, wherein R²⁰ is 1 to 5 substituents each independently selected from:
a) halo;
b) R⁷, wherein R⁷ is selected from H, (C₁₋₆)alkyl, (C₁₋₆)haloalkyl, (C₃₋₇)cycloalkyl, aryl and Het;
   wherein each of the aryl and Het is optionally substituted with 1 to 3 substituents each independently selected from:
   i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
   ii) (C₁₋₆)alkyl optionally substituted with —OH, or —O—(C₁₋₆)alkyl; and
   iii) aryl or Het, wherein each of the aryl and Het is optionally substituted with halo or (C₁₋₆)alkyl;
c) —C(═O)—R⁷, —C(═O)—O—R⁷, —O—R⁷, —S—R⁷, —SO—R⁷, —SO₂—R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
   wherein R⁷ is as defined above;
d) aryl-(C₁₋₆)alkyl or Het-(C₁₋₆)alkyl,
   wherein each of the aryl and Het is optionally substituted with 1 to 3 substituents each independently selected from:
   i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
   ii) (C₁₋₆)alkyl optionally substituted with —OH or —O—(C₁₋₆)alkyl; and

iii) aryl or Het, wherein each of the aryl and Het is optionally substituted with halo or (C₁₋₆)alkyl; and
e) —N(R⁸)R⁹, —C(═O)—N(R⁸)R⁹, —SO₂—N(R⁸)R⁹, or —(C₁₋₆)alkylene-N(R⁸)R⁹, wherein
   R⁸ is in each instance independently selected from H and (C₁₋₆)alkyl; and
   R⁹ is in each instance independently selected from R⁷, —(C₃₋₇)cycloalkyl-(C₁₋₆)alkyl, —C(═O)—R¹⁰, —C(═O)OR¹⁰ and —C(═O)N(H)R¹⁰;
   wherein R⁷ is as defined above;
   wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
   wherein R¹⁰ is in each instance independently selected from (C₁₋₆)alkyl, and Het, wherein said Het is optionally substituted with (C₁₋₆)alkyl; and
   wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
R⁵ is selected from H, (C₁₋₆)alkyl, (C₃₋₇)cycloalkyl, and Het; the (C₁₋₆)alkyl and Het each being optionally substituted with 1 to 4 substituents each independently selected from (C₁₋₆)alkyl, —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂, and —SO₂(C₁₋₆)alkyl; and
R⁶ is selected from (C₅₋₇)cycloalkyl and aryl;
the (C₅₋₇)cycloalkyl and aryl each being optionally substituted with 1 to 5 substituents each independently selected from halo, (C₁₋₆)alkyl, (C₁₋₆)haloalkyl, —OH, —SH, —O—(C₁₋₄)alkyl and —S—(C₁₋₄)alkyl;
wherein Het is a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S;
   or a salt or ester thereof.

Examples of most preferred compounds according to this invention are each single compound listed in the following Tables 1, 2 and 3.

In general, all tautomeric and isomeric forms and mixtures thereof, for example, individual geometric isomers, stereoisomers, atropisomers, enantiomers, diastereomers, racemates, racemic or non-racemic mixtures of stereoisomers, mixtures of diastereomers, or mixtures of any of the foregoing forms of a chemical structure or compound is intended, unless the specific stereochemistry or isomeric form is specifically indicated in the compound name or structure.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of the present invention from this disclosure and the knowledge in the art.

Preparation of pure stereoisomers, e.g. Enantiomers and diastereomers, or mixtures of desired enantiomeric excess (ee) or enantiomeric purity, are accomplished by one or more of the many methods of (a) separation or resolution of enantiomers, or (b) enantioselective synthesis known to those of skill in the art, or a combination thereof. These resolution methods generally rely on chiral recognition and include, for example, chromatography using chiral stationary phases, enantioselective host-guest complexation, resolution or synthesis using chiral auxiliaries, enantioselective synthesis, enzymatic and nonenzymatic kinetic resolution, or spontaneous enantioselective crystallization. Such methods are disclosed generally in Chiral Separation Techniques: A Practical Approach (2nd Ed.), G. Subramanian (ed.), Wiley-VCH, 2000; T. E. Beesley and R. P. W. Scott, Chiral Chromatography, John Wiley & Sons, 1999; and Satinder Ahuja, Chiral Separations by Chromatography, Am. Chem. Soc., 2000, herein incorporated by reference. Furthermore, there are equally well-known methods for the quantitation of enantiomeric excess or purity, for example, GC, HPLC, CE, or NMR, and assignment of absolute configuration and conformation, for example, CD ORD, X-ray crystallography, or NMR.

The compounds according to the present invention are inhibitors of the hepatitis C virus NS5B RNA-dependent RNA polymerase and thus may be used to inhibit replication of hepatitis C viral RNA.

A compound according to the present invention may also be used as a laboratory reagent or a research reagent. For example, a compound of the present invention may be used as positive control to validate assays, including but not limited to surrogate cell-based assays and in vitro or in vivo viral replication assays.

Compounds according to the present invention may also be used as probes to study the hepatitis C virus NS5B polymerase, including but not limited to the mechanism of action of the polymerase, conformational changes undergone by the polymerase under various conditions and interactions with entities which bind to or otherwise interact with the polymerase.

Compounds of the invention used as probes may be labelled with a label which allows recognition either directly or indirectly of the compound such that it can be detected, measured and quantified. Labels contemplated for use with the compounds of the invention include, but are not limited to, fluorescent labels, chemiluminescent labels, colorimetric labels, enzymatic markers, radioactive isotopes, affinity tags and photoreactive groups.

Compounds of the invention used as probes may also be labelled with an affinity tag whose strong affinity for a receptor can be used to extract from a solution the entity to which the ligand is attached. Affinity tags include but are not limited to biotin or a derivative thereof, a histidine polypeptide, a polyarginine, an amylose sugar moiety or a defined epitope recognizable by a specific antibody.

Furthermore, compounds of the invention used as probes may be labelled with a photoreactive group which is transformed, upon activation by light, from an inert group to a reactive species, such as a free radical. Photoreactive groups include but are not limited to photoaffinity labels such as benzophenone and azide groups.

Furthermore, a compound according to the present invention may be used to treat or prevent viral contamination of materials and therefore reduce the risk of viral infection of laboratory or medical personnel or patients who come in contact with such materials (e.g. blood, tissue, surgical instruments and garments, laboratory instruments and garments, and blood collection apparatuses and materials).

### Pharmaceutical Composition

Compounds of the present invention may be administered to a mammal in need of treatment for hepatitis C viral infection as a pharmaceutical composition comprising a therapeutically effective amount of a compound according to the invention or a pharmaceutically acceptable salt or ester thereof; and one or more conventional non-toxic pharmaceutically-acceptable carriers, adjuvants or vehicles. The specific formulation of the composition is determined by the solubility and chemical nature of the compound, the chosen route of administration and standard pharmaceutical practice. The pharmaceutical composition according to the present invention may be administered orally or systemically.

For oral administration, the compound, or a pharmaceutically acceptable salt or ester thereof, can be formulated in any orally acceptable dosage form including but not limited to aqueous suspensions and solutions, capsules, powders, syrups, elixirs or tablets. For systemic administration, including but not limited to administration by subcutaneous, intracutaneous, intravenous, intramuscular, intra-articular, intrasynovial, intrasternal, intrathecal, and intralesional injection or infusion techniques, it is preferred to use a solution of the compound, or a pharmaceutically acceptable salt or ester thereof, in a pharmaceutically acceptable sterile aqueous vehicle.

Pharmaceutically acceptable carriers, adjuvants, vehicles, excipients and additives as well as methods of formulating pharmaceutical compositions for various modes of administration are well-known to those of skill in the art and are described in pharmaceutical texts such as Remington: The Science and Practice of Pharmacy, 21st Edition, Lippincott Williams & Wilkins, 2005; and L. V. Allen, N. G. Popovish and H. C. Ansel, Pharmaceutical Dosage Forms and Drug Delivery Systems, 8th ed., Lippincott Williams & Wilkins, 2004, herein incorporated by reference.

The dosage administered will vary depending upon known factors, including but not limited to the activity and pharmacodynamic characteristics of the specific compound employed and its mode, time and route of administration; the age, diet, gender, body weight and general health status of the recipient; the nature and extent of the symptoms; the severity and course of the infection; the kind of concurrent treatment; the frequency of treatment; the effect desired; and the judgment of the treating physician. In general, the compound is most desirably administered at a dosage level that will generally afford antivirally effective results without causing any harmful or deleterious side effects.

A daily dosage of active ingredient can be expected to be about 0.01 to about 200 milligrams per kilogram of body weight, with the preferred dose being about 0.1 to about 50 mg/kg. Typically, the pharmaceutical composition of this invention will be administered from about 1 to about 5 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

### Combination Therapy

Combination therapy is contemplated wherein a compound according to the invention, or a pharmaceutically acceptable salt or ester thereof, is co-administered with at least one additional antiviral agent. The additional agents may be combined with compounds of this invention to create a single dosage form. Alternatively these additional agents may be separately administered, concurrently or sequentially, as part of a multiple dosage form.

When the pharmaceutical composition of this invention comprises a combination of a compound according to the invention, or a pharmaceutically acceptable salt or ester thereof, and one or more additional antiviral agent, both the compound and the additional agent should be present at dosage levels of between about 10 to 100%, and more preferably between about 10 and 80% of the dosage normally administered in a monotherapy regimen. In the case of a synergistic interaction between the compound of the invention and the additional antiviral agent or agents, the dosage of any or all of the active agents in the combination may be reduced compared to the dosage normally administered in a monotherapy regimen.

Antiviral agents contemplated for use in such combination therapy include agents (compounds or biologicals) that are effective to inhibit the formation and/or replication of a virus in a mammal, including but not limited to agents that interfere with either host or viral mechanisms necessary for the formation and/or replication of a virus in a mammal. Such agents can be selected from another anti-HCV agent; an HIV inhibitor; an HAV inhibitor; and an HBV inhibitor.

Other anti-HCV agents include those agents that are effective for diminishing or preventing the progression of hepatitis C related symptoms or disease. Such agents include but are not limited to immunomodulatory agents, inhibitors of HCV NS3 protease, other inhibitors of HCV polymerase, inhibitors of another target in the HCV life cycle and other anti-HCV agents, including but not limited to ribavirin, amantadine, levovirin and viramidine.

Immunomodulatory agents include those agents (compounds or biologicals) that are effective to enhance or potentiate the immune system response in a mammal. Immunomodulatory agents include, but are not limited to, inosine monophosphate dehydrogenase inhibitors such as VX-497 (merimepodib, Vertex Pharmaceuticals), class I interferons, class II interferons, consensus interferons, asialo-interferons pegylated interferons and conjugated interferons, including but not limited to interferons conjugated with other proteins including but not limited to human albumin. Class I interferons are a group of interferons that all bind to receptor type I, including both naturally and synthetically produced class I interferons, while class II interferons all bind to receptor type II. Examples of class I interferons include, but are not limited to, α-, β-, δ-, ω-, and τ-interferons, while examples of class II interferons include, but are not limited to, γ-interferons.

Inhibitors of HCV NS3 protease include agents (compounds or biologicals) that are effective to inhibit the function of HCV NS3 protease in a mammal. Inhibitors of HCV NS3 protease include, but are not limited to, those compounds described in WO 99/07733, WO 99/07734, WO 00/09558, WO 00/09543, WO 00/59929, WO 03/064416, WO 03/064455, WO 03/064456, WO 2004/030670, WO 2004/037855, WO 2004/039833, WO 2004/101602, WO 2004/101605, WO 2004/103996, WO 2005/028501, WO 2005/070955, WO 2006/000085 (all by Boehringer Ingelheim), WO 02/060926, WO 03/053349, WO 03/099274, WO 03/099316, WO 2004/032827, WO 2004/043339, WO 2004/094452, WO 2005/046712, WO 2005/051410, WO 2005/054430 (all by BMS), WO 2004/072243, WO 2004/093798, WO 2004/113365, WO 2005/010029 (all by Enanta), WO 2005/037214 (Intermune), WO 01/77113, WO 01/81325, WO 02/08187, WO 02/08198, WO 02/08244, WO 02/08256, WO 02/48172, WO 03/062228, WO 03/062265, WO 2005/021584, WO 2005/030796, WO 2005/058821, WO 2005/051980, WO 2005/085197, WO 2005/085242, WO 2005/085275, WO 2005/087721, WO 2005/087725, WO 2005/087730, WO 2005/087731, WO 2005/107745 and WO 2005/113581 (all by Schering), WO 2006/119061, WO 2007/016441, WO 2007/015855, WO 2007/015787 (all by Merck); and the candidates VX-950, ITMN-191 and SCH-503034.

Inhibitors of HCV polymerase include agents (compounds or biologicals) that are effective to inhibit the function of an HCV polymerase. Such inhibitors include, but are not limited to, non-nucleoside and nucleoside inhibitors of HCV NS5B polymerase. Examples of inhibitors of HCV polymerase include but are not limited to those compounds described in: WO 02/04425, WO 03/007945, WO 03/010140, WO 03/010141, WO 2004/064925, WO 2004/065367, WO 2005/080388, WO 2006/007693, WO 2007/019674, WO2007/087717 (all by Boehringer Ingelheim), WO 01/47883 (Japan Tobacco), WO 03/000254 (Japan Tobacco), WO 03/026587 (BMS), WO 2004/087714 (IRBM), WO 2005/012288 (Genelabs), WO 2005/014543 (Japan Tobacco), WO 2005/049622 (Japan Tobacco), WO 2005/121132 (Shionogi), WO 2005/080399 (Japan Tobacco), WO 2006/052013 (Japan Tobacco), WO 2006/119646 (Virochem Pharma), WO 2007/039146 (SmithKline Beecham), WO 2005/021568 (Biota), WO 2006/094347 (Biota) and the candidates HCV 796 (ViroPharma/Wyeth), R-1626, R-7128 and R-1656 (Roche), VCH-759 (Virochem), NM 283 (Idenix/Novartis), GSK625433 (GSK), GS9190 (Gilead), MK-608 (Merck) and PF868554 (Pfizer).

Inhibitors of another target in the HCV life cycle include agents (compounds or biologicals) that are effective to inhibit the formation and/or replication of HCV other than by inhibiting the function of the HCV NS3 protease or HCV polymerase. Such agents may interfere with either host or HCV viral mechanisms necessary for the formation and/or replication of HCV. Inhibitors of another target in the HCV life cycle include, but are not limited to, entry inhibitors, agents that inhibit a target selected from a helicase, a NS2/3 protease and an internal ribosome entry site (IRES) and agents that interfere with the function of other viral targets including but not limited to an NS5A protein and an NS4B protein.

It can occur that a patient may be co-infected with hepatitis C virus and one or more other viruses, including but not limited to human immunodeficiency virus (HIV), hepatitis A virus (HAV) and hepatitis B virus (HBV). Thus also contemplated is combination therapy to treat such co-infections by co-administering a compound according to the present invention with at least one of an HIV inhibitor, an HAV inhibitor and an HBV inhibitor.

HIV inhibitors include agents (compounds or biologicals) that are effective to inhibit the formation and/or replication of HIV. This includes but is not limited to agents that interfere with either host or viral mechanisms necessary for the formation and/or replication of HIV in a mammal. HIV inhibitors include, but are not limited to:
NRTIs (nucleoside or nucleotide reverse transcriptase inhibitors; including but not limited to zidovudine, didanosine, zalcitabine, stavudine, lamivudine, emtricitabine, abacavir, and tenofovir);
NNRTIs (non-nucleoside reverse transcriptase inhibitors; including but not limited to nevirapine, delavirdine, efavirenz, capravirine, etravirine, rilpivirine and BILR 355);
protease inhibitors (including but not limited to ritonavir, tipranavir, saquinavir, nelfinavir, indinavir, amprenavir, fosamprenavir, atazanavir, lopinavir, VX-385 and TMC-114);
entry inhibitors including but not limited to CCR5 antagonists (including but not limited to maraviroc (UK-427,857) and TAK-652), CXCR4 antagonists (including but not limited to AMD-11070), fusion inhibitors (including but not limited to enfuvirtide (T-20)) and others (including but not limited to BMS-488043);
integrase inhibitors (including but not limited to MK-0518, c-1605, BMS-538158 and GS 9137);
TAT inhibitors;
maturation inhibitors (including but not limited to PA-457); and
immunomodulating agents (including but not limited to levamisole).

HAV inhibitors include agents (compounds or biologicals) that are effective to inhibit the formation and/or replication of HAV. This includes but is not limited to agents that interfere with either host or viral mechanisms necessary for the formation and/or replication of HAV in a mammal. HAV inhibitors include but are not limited to Hepatitis A vaccines.

HBV inhibitors include agents (compounds or biologicals) that are effective to inhibit the formation and/or replication of HBV in a mammal. This includes but is not limited to agents that interfere with either host or viral mechanisms necessary for the formation and/or replication of HBV in a mammal. HBV inhibitors include, but are not limited to, agents that inhibit the HBV viral DNA polymerase and HBV vaccines.

Therefore, according to one embodiment, the pharmaceutical composition of this invention additionally comprises a therapeutically effective amount of one or more antiviral agents.

A further embodiment provides the pharmaceutical composition of this invention wherein the one or more antiviral agent comprises at least one other anti-HCV agent.

According to a more specific embodiment of the pharmaceutical composition of this invention, the at least one other anti-HCV agent comprises at least one immunomodulatory agent.

According to another more specific embodiment of the pharmaceutical composition of this invention, the at least one other anti-HCV agent comprises at least one other inhibitor of HCV polymerase.

According to yet another more specific embodiment of the pharmaceutical composition of this invention, the at least one other anti-HCV agent comprises at least one inhibitor of HCV NS3 protease.

According to still another more specific embodiment of the pharmaceutical composition of this invention, the at least one other anti-HCV agent comprises at least one inhibitor of another target in the HCV life cycle.

### EXAMPLES

Other features of the present invention will become apparent from the following non-limiting examples which illustrate, by way of example, the principles of the invention. As is well known to a person skilled in the art, reactions are performed in an inert atmosphere (including but not limited to nitrogen or argon) where necessary to protect reaction components from air or moisture. Temperatures are given in degrees Celsius (° C.). Solution percentages and ratios express a volume to volume relationship, unless stated otherwise. Flash chromatography is carried out on silica gel (SiO₂) according to the procedure of W. C. Still et al., J. Org. Chem., (1978), 43, 2923. Mass spectral analyses are recorded using electrospray mass spectrometry. Purification on a combiflash is performed using an Isco Combiflash (column cartridge SiO₂). Preparative HPLC is carried out under standard conditions using a SunFire™ Prep C18 OBD 5 μM reverse phase column, 19×50 mm and a linear gradient (20 to 98%) employing 0.1% TFA/acetonitrile and 0.1% TFA/water as solvents. Compounds are isolated as TFA salts when applicable. Analytical HPLC is carried out under standard conditions using a Combiscreen™ ODS-AQ C18 reverse phase column, YMC, 50×4.6 mm i.d., 5 μM, 120 Å at 220 nM, elution with a linear gradient as described in the following table (Solvent A is 0.06% TFA in H₂O; solvent B is 0.06% TFA in CH₃CN):

| | Flow | Solvent | Solvent |
|---|---|---|---|
| Time (min) | (mL/min) | A (%) | B (%) |
| 0 | 3.0 | 95 | 5 |
| 0.5 | 3.0 | 95 | 5 |
| 6.0 | 3.0 | 50 | 50 |
| 10.5 | 3.5 | 0 | 100 |

Abbreviations or symbols used herein include:
Ac: acetyl;
AcOH: acetic acid;
Bn: benzyl (phenylmethyl);
BOC or Boc: tert-butyloxycarbonyl;
Bu: butyl;
n-BuLi: n-butyllithium;
n-BuOAc: n-butylacetate;
m-CPBA: meta-chloroperbenzoic acid;
DBU: 1,8-diazabicyclo[5.4.0]undec-7-ene;
DCE: dichloroethane;
DCM: dichloromethane;
DEAD: diethyl azodicarboxylate;
DIAD: diisopropyl azodicarboxylate;
DIPEA: diisopropylethylamine;
DMAP: 4-dimethylaminopyridine;
DMF: N,N-dimethylformamide;
DMSO: dimethylsulfoxide;
EC₅₀: 50% effective concentration;
Et: ethyl;
Et₃N: triethylamine;
Et₂O: diethyl ether;
EtOAc: ethyl acetate;
EtOH: ethanol;
Hex: hexane;
HPLC: high performance liquid chromatography;
IC₅₀: 50% inhibitory concentration;
ⁱPr or i-Pr: 1-methylethyl (iso-propyl);
LDA: lithium diisoproylamide;
Me: methyl;
MeCN: acetonitrile;
MeI: iodomethane;
MeOH: methanol;
MS: mass spectrometry;
NADPH: Nicotinamide adenine dinucleotide phosphate (reduced form);
NaHB(OAc)₃: sodium triactoxyborohydride;
NaHMDS: sodium hexamethyldisilazane;
NIS: N-iodosuccinamide;
NMR: nuclear magnetic resonance spectroscopy;
Ph: phenyl;
Pr: propyl;
RT: room temperature (approximately 18° C. to 25° C.);
tert-butyl or t-butyl: 1,1-dimethylethyl;
TBABr: tetrabutylammonium bromide;
TBAF: tetrabutylammonium fluoride;
TFA: trifluoroacetic acid;
THF: tetrahydrofuran;
TLC: thin layer chromatography.

### Example 1A

### Preparation of Intermediate 1a9

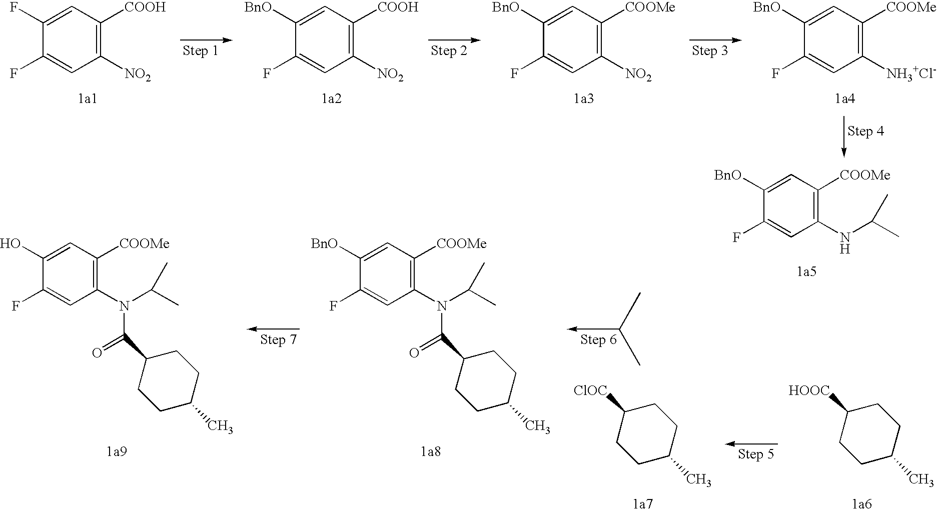

### Step 1:

4,5-Difluoro-2-nitrobenzene lal (73 g, 359 mmol) is diluted in anhydrous THF (2 L) under argon. Benzyl alcohol (80.8 mL, 800 mmol) is added and the mixture is chilled to 0° C. Sodiumbis(trimethylsilyl)amide (1.0 M in THF, 800 mL, 800 mmol) is added dropwise. After stirring for one hour, the mixture is partitioned between saturated aqueous NH₄Cl and EtOAc. The organic phase is collected and dried over sodium sulfate. The mixture is filtered and concentrated. The resulting solid 1a2 is washed with cold EtOAc and dried.

### Step 2:

Carboxylic acid 1a2 (112.8 g, 384 mmol) is diluted in anhydrous DMF (2 L). Potassium carbonate (108.1 g, 775 mmol) is added and the mixture is chilled to 0° C. Iodomethane (110 g, 775 mmol) is added dropwise and after 2 hours the reaction is quenched by the addition of saturated aqueous ammonium chloride. The aqueous solution is extracted with ethyl acetate (×2). The combined organic extracts are then washed with water and brine before being dried with MgSO₄. Removal of solvent results in methyl ester 1a3.

### Step 3a:

The nitro intermediate 1a3 (63.8 g, 212 mmol) is diluted in THF (1 L). Aqueous hydrochloric acid (1 M, 500 mL) is added followed by tin powder (55 g, 467 mmol). The mixture is stirred for 2 hours at RT. The reaction mixture is then diluted in EtOAc and pH of the mixture is adjusted to 7 by the addition of 1 N NaOH. The organic phase is separated then washed with water and brine. The organic phase is then dried over NaSO₄ and solvent is removed to afford aniline.

### Step 3b:

The aniline (97.1 g, 377 mmol) is combined with anhydrous Et₂O (1 L) and then is treated by the slow addition of HCl (2 M in ether, 2 L). The resulting hydrochloride salt 1a4 is collected by filtration and washed with excess ether.

### Step 4:

Reference: Abdel-Magid, A. F.; Carson, K. G.; Harris, B. D.; Maryanoff, C. A.; Shah, R. D. J. Org. Chem. 1996, 61, 3849.

The aniline hydrochloride salt 1a4 (105.4 g, 358 mmol) is combined with anhydrous DCM (2.8 L). 2-Methoxypropene (103.3 g, 1430 mmol) is added followed by sodium triacetoxyborohydride (151.8 g, 716 mmol). The mixture is stirred overnight at RT, then diluted in EtOAc and washed with saturated aqueous NaHCO₃ and brine. The organic phase is dried over Na₂SO₄, filtered, then concentrated under reduced pressure. The resulting solid is recrystallized from EtOAc/Hex to afford isopropylaniline 1a5.

### Step 5:

To a mixture of compound 1a6 (43.4 g, 305 mmol) and anhydrous CH₂Cl₂ (400 mL) under an Ar atmosphere at RT is added (COCl)₂ (53.2 mL, 610 mmol) in CH₂Cl₂ (305 mL) dropwise over 1 hour at RT. The mixture is stirred for 1 hour at RT and anhydrous DMF (1 mL) is added dropwise. The mixture is stirred overnight at RT and concentrated under reduced pressure. The residue is diluted with pentane and filtered. The filtrate is twice concentrated under reduced pressure, diluted with pentane and filtered, then concentrated under reduced pressure to provide acid chloride 1a7.

### Step 6:

The i-Pr-aniline 1a5 (41.1 g, 138 mmol) is combined with anhydrous pyridine (60 mL) and anhydrous DCM (60 mL) under argon. The acid chloride 1a7 (34 mL, 211 mmol) is added followed by DMAP (3.5 g, 28 mmol) and the mixture is heated to 60° C. and stirred overnight. The mixture is then allowed to cool before being diluted in EtOAc. The organic phase is washed with aqueous 2 M HCl (×2), NaHCO₃ (×2) and brine, then dried over NaSO₄. The solvent is removed under reduced pressure. The resulting oil is treated with DCM/Heptane to obtain solid 1a8.

### Step 7:

Benzyl ether 1a8 (20.0 g, 45.3 mmol) is dissolved in a 1:1 mixture of MeOH and EtOAc (500 mL) in a Parr Hydrogenator™. 10% Pd(OH)₂/C (2 g) is added and the vessel is pressurized with 30 psi of H₂ and agitated overnight. The mixture is filtered through a pad of celite, then concentrated in vacuo to afford phenol 1a9.

### Example 2A

### Preparation of Intermediate 2a4

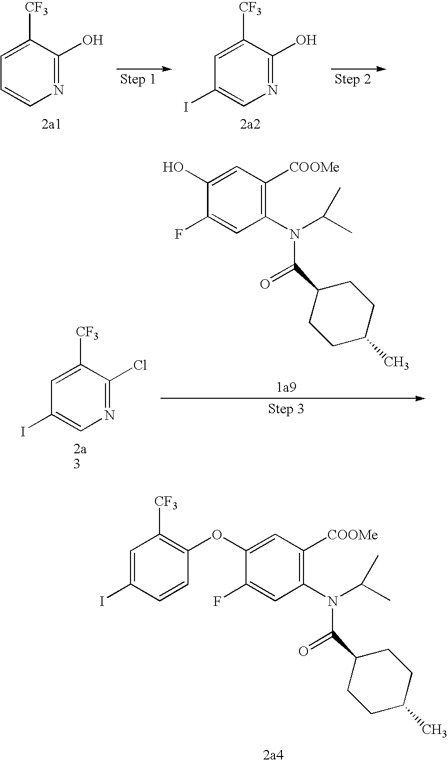

### Step 1:

To a mixture of 2-hydroxy-3-trifluoromethylpyridine 2a1 (39.01 g, 239 mmol) and anhydrous DMF (800 mL) under Ar is added N-iodosuccinimide (4.89 g, 244 mmol) and anhydrous K₂CO₃ (33.72 g, 244 mmol). The mixture is allowed to stir at 60° C. for about 3 hours. The mixture is cooled to ambient temperature, filtered and concentrated under reduced pressure. The residue is dissolved in DCM (1 L) and the organic phase is washed with brine. The aqueous phase is adjusted to pH 4 by the addition of 2M HCl, then extracted with DCM (1 L). The combined organic extracts are washed with brine (2 L) and dried over Na₂SO₄. The mixture is concentrated to ˜300 mL and cooled overnight in a fridge. The precipitated solid is removed by filtration and dried to provide iodide 2a2.

### Step 2:

A mixture of compound 2a2 (115.7 g, 400 mmol) and PhPOCl₂ (668.6 g, 343 mmol) under N₂ is stirred at 136° C. overnight, then cooled to RT and added slowly to 3 L of crushed ice. The aqueous mixture is adjusted to pH 6 and filtered. The aqueous filtrate is extracted with DCM (3 L) then the organic phase is washed with saturated NaHCO₃ and brine, dried over Na₂SO₄, filtered and concentrated to provide chloropyridine 2a3.

### Step 3:

To a mixture of phenol 1a9 (16.6 g, 47.2 mmol) in DMSO (220 mL) under an Ar atmosphere is added anhydrous K₂CO₃ (17.3 g, 125 mmol). Chloropyridine 2a3 (14.4 g, 56.7 mmol) is added and the mixture is heated to 100° C. and stirred for about 4 hours. The mixture is diluted in EtOAc (500 mL), and then washed with saturated aqueous NH₄Cl (500 mL×2) and brine (200 mL). The organic phase is dried over Na₂SO₄, filtered and concentrated under reduced pressure. Purification is performed by flash chromatography (10% EtOAc in Hex) to afford ether 2a4.

### Example 3A

### Preparation of Intermediate 3a4 & Compound 1001

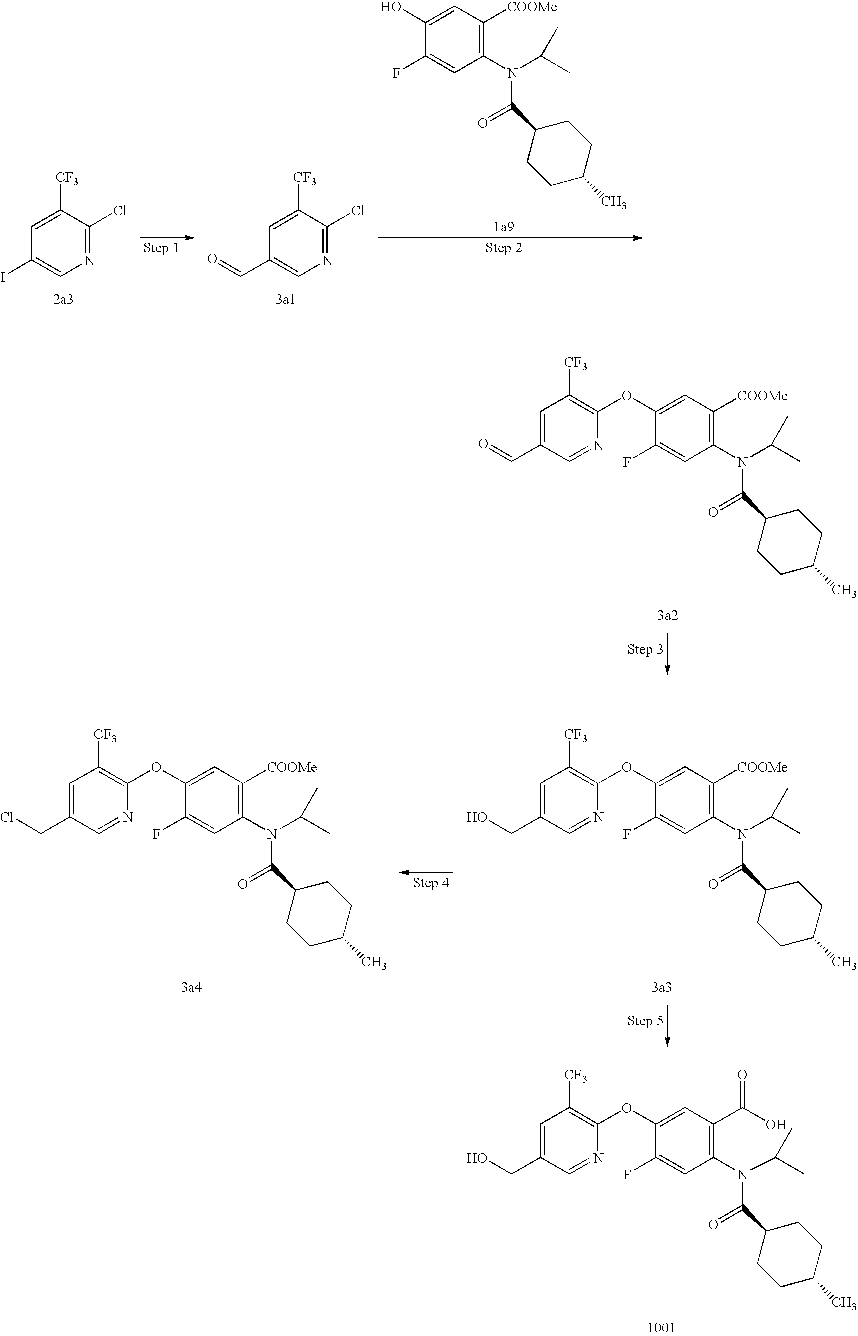

### Step 1:

Iodide 2a3 (10 g, 32.5 mmol) is combined with a 1:3 mixture of anhydrous THF and anhydrous toluene (100 mL) under an Ar atmosphere. The mixture is cooled to −78° C. then n-BuLi (1.6 M in hexanes, 24 mL, 38.4 mmol) is added slowly by syringe over 40 minutes. Stirring is continued for about 1 hour before ethylformate (3.2 mL, 39.7 mmol) in THF (10 mL) is added over a period of about 40 minutes. The mixture is stirred for 1 hour before being quenched by the addition of 2 M HCl. The mixture is partitioned between EtOAc and saturated aqueous NaHCO₃. The organic phase is washed with brine and dried over Na₂SO₄. The mixture is filtered and concentrated under reduced pressure. Purification is performed by flash chromatography where the silica gel is pre-treated with 3% NEt₃ in hexanes then eluted with 1:1 EtOAc/Hex to isolate aldehyde 3a2.

### Step 2:

Aldehyde 3a2 is coupled with 1a9 using S_{N}Ar reaction conditions described in example 2A step 3.

### Step 3:

The aldehyde 3a2 (8.9 g, 16 mmol) is combined with methanol (50 mL) in a round bottom flask equipped with a stirrer. Sodium borohydride (1.22 g, 32 mmol) is added and the mixture is stirred under Ar at RT for about 4 hours. The mixture is diluted with EtOAc (300 mL), and washed with 1N HCl (200 mL), saturated aqueous NaHCO₃ (200 mL) and brine (100 mL). The organic phase is dried over Na₂SO₄, filtered and the solvent is removed to provide alcohol 3a3 that is used without purification in the next step.

### Step 4:

The crude alcohol 3a3 (10.65 g, 16.1 mmol) is combined with anhydrous DCM (200 mL) and anhydrous DMF (4 mL) under an Ar atmosphere. Thionyl chloride (3.83 mL, 32.2 mmol) is added to the mixture which is then stirred for about 4 hours at RT. The mixture is diluted with EtOAc (600 mL) and then washed with 1N HCl (100 mL), saturated aqueous NaHCO₃ (100 mL) and brine (100 ml). The organic phase is dried over Na₂SO₄, filtered and the solvent is removed. The residue is subjected to flash chromatography (silica, 95:5 to 8:2 Hex/EtOAc) to afford benzyl chloride 3a4. This is further purified by crystallization form hexane/DCM.

### Step 5:

Saponification of 3a3 provides compound 1001 using conditions analogous to example 4A step 1(b).

### Example 4A

### Method A

### Preparation of Compound 1002

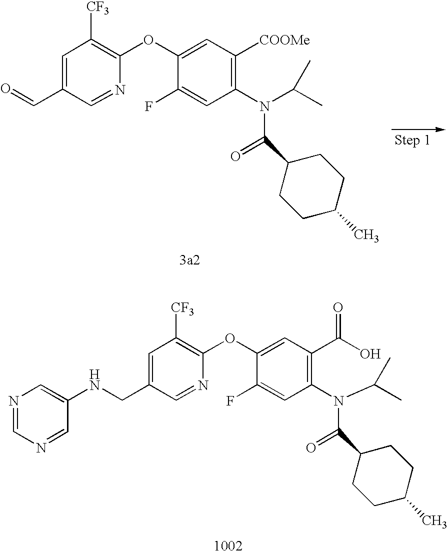

### Step 1:

(a) Aldehyde 3a2 (50 mg, 0.10 mmol) is combined with aminopyrazine (38 mg, 0.40 mmol) in DMF (0.5 mL). To the mixture is added HCl (4.0 M in dioxane, 50 μL, 0.20 mmol) followed by NaCNBH₃ (14 mg, 0.23 mmol) and the mixture is stirred at RT for about 1 hour. (b) Methanol (1 mL) and MeCN (0.5 mL) are added, followed by NaOH (2.5 N, 250 μL, 1.0 mmol). The mixture is stirred for about 2 hours at 50° C. before being acidified with AcOH and injected onto the preparative HPLC to isolate compound 1002.

### Example 5A

### Method B

### Preparation of Compound 1006

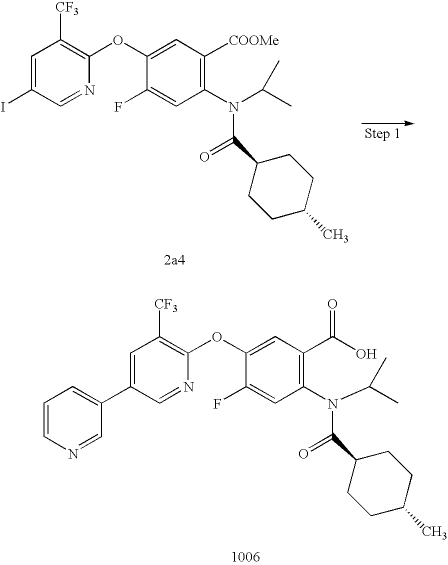

### Step 1:

To a mixture of iodide 2a4 (45 mg, 0.07 mmol), 3-pyridylboronic acid (22 mg, 0.18 mmol), tetrakis(triphenylphosphino) palladium (0) (17 mg, 0.01 mmol) and degassed DMF (2 mL) is added 2 M aqueous Na₂CO₃ (0.14 mL, 0.29 mmol). The mixture is heated to 100° C. and stirred for about 1 hour. The mixture is then allowed to cool at ambient temperature and water (0.3 mL), MeOH (0.3 mL) and aqueous NaOH (10 M, 0.15 mL, 1.5 mmol) are added. The mixture is acidified by the addition of TFA and the mixture is filtered and injected onto a preparative HPLC to isolate compound 1006.

### Example 6A

### Preparation of Compound 1008 & 1009

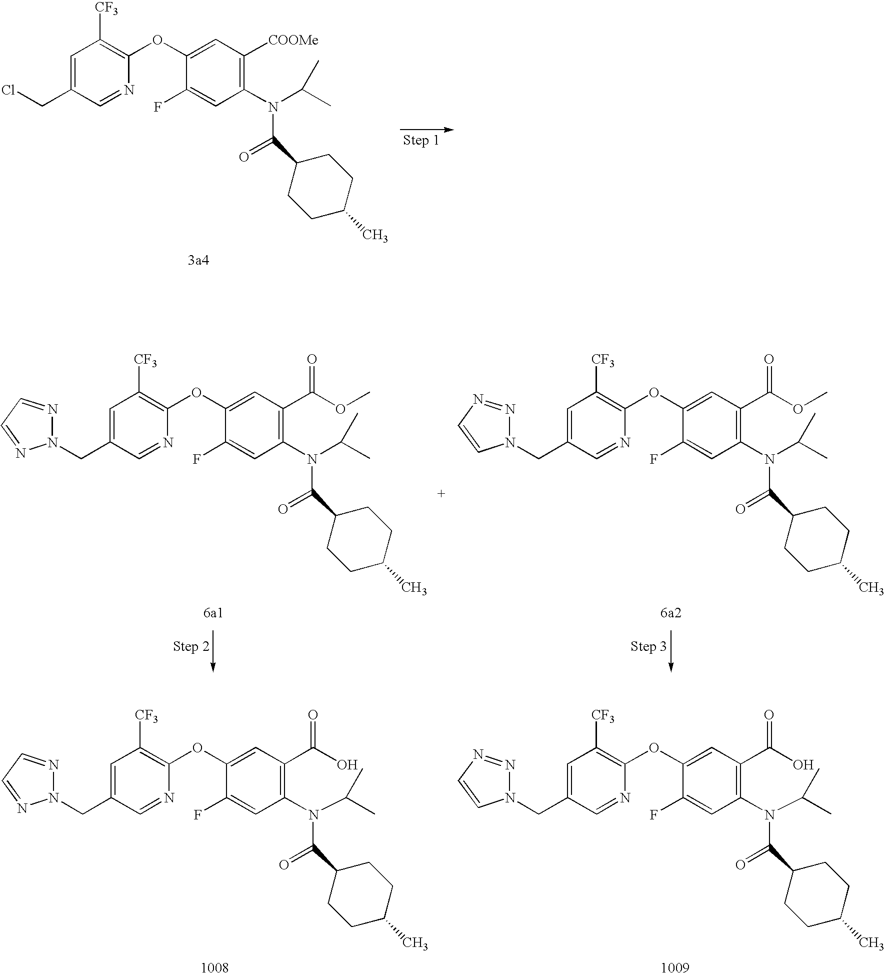

### Step 1:

Triazole (17 μL, 0.30 mmol) is added to a chilled (0° C.) mixture of NaH (60% dispersion in mineral oil, 11 mg, 0.28 mmol) in DMF (1 mL). After bubbling ceases, the mixture is transferred via cannula into a vessel containing benzyl chloride 3a4 (110 mg, 0.20 mmol) in DMF (1 mL+0.5 mL wash). The mixture is stirred for about 1 hour at 0° C. before being allowed to warm to RT and stirring continues for 5 hours. The reaction mixture is diluted in EtOAc and washed with 0.5 N aqueous KHSO₄, saturated aqueous NaHCO₃ and brine. The organic phase is dried with MgSO₄ and filtered. Silica gel is added to the solution and then the mixture is concentrated. The dry packed compound on silica is purified by combiflash to afford the isomeric benzylic triazoles 6a1 and 6a2.

### Step 2:

Ester 6a1 (53 mg, 0.09 mmol) is combined with THF (1 mL) and MeOH (0.2 mL). Sodium hydroxide (10 N, 90 μL, 0.90 mmol) is added and the mixture is stirred at RT overnight. The mixture is acidified with TFA (83 μL, 1.08 mmol) then concentrated. The residue is taken-up in DMSO and injected onto the preparative HPLC for purification to provide compound 1008.

### Step 3:

Starting with benzylic triazole 6a2 and following the protocol described in step 2, compound 1009 is generated.

### Example 7A

### Preparation of Compound 1011

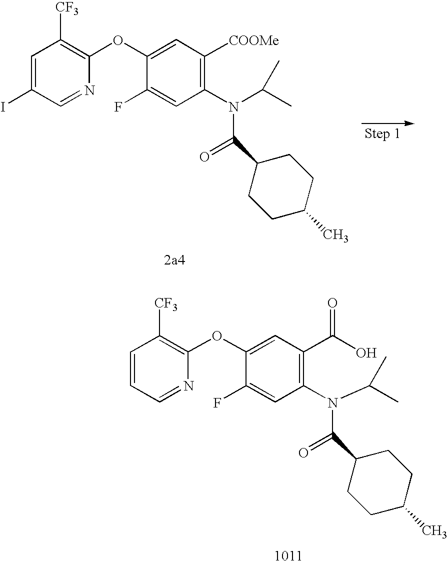

### Step 1:

Iodide 2a4 (45 mg, 0.07 mmol) is combined with 10% Pd/C (12 mg) in MeOH (3 mL). The vessel is purged with H₂ then is stirred under 1 atm of H₂ overnight. The mixture is filtered through celite then concentrated under reduced pressure. To a mixture of the residue in DMSO (2 mL), MeOH (1 mL) and water (75 μL) is added NaOH (10 N, 75 μL, 0.75 mmol). The mixture is stirred at RT overnight. The mixture is acidified with TFA (83 μL, 1.08 mmol) then concentrated. The residue is taken-up in DMSO and injected onto the preparative HPLC for purification to provide compound 1011.

### Example 8

### Method C

### Preparation of Compound 1012

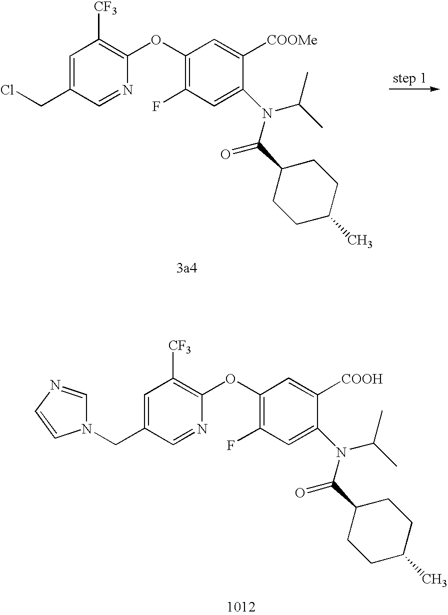

A mixture of intermediate 3a4 (110 mg, 0.2 mmol), imidazole (20 mg, 0.3 mmol), Cs₂CO₃ (100 mg, 0.30 mmol), KI (6 mg, 0.04 mmol) and MgSO₄ (70 mg, 0.58 mmol) in DMF (2 mL) is agitated on a J-Kem® orbital shaker (300 rpm) at 70° C. overnight. The mixture is cooled to ambient temperature, filtered and washed with DMSO (0.5 mL). Aqueous NaOH (5 N, 0.4 mL, 2.0 mmol) is added and the mixture is stirred at RT for 2 hours. The mixture is acidified with AcOH, then is purified by preparative HPLC to isolate compound 1012.

### Example 8

Method D

### Preparation of Compound 1017

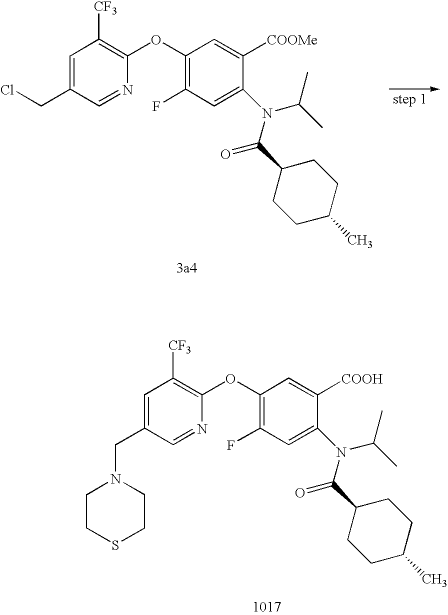

Step 1:

A mixture of intermediate 3a4 (110 mg, 0.3 mmol), thiomorpholine (30 mg, 0.3 mmol) and Et₃N (42 μL, 0.3 mmol) in THF (2 mL) is agitated on a J-Kem® orbital shaker (300 rpm) at 70° C. overnight. The mixture is concentrated under reduced pressure using a Savant™ speed-vac then taken up in DMSO (1 mL). Aqueous NaOH (5 N, 0.4 mL, 2.0 mmol) is added and the mixture is stirred at RT for about 2 hours. The mixture is acidified with AcOH and purified by preparative HPLC to isolate compound 1017.

### Example 9

### Preparation of Compound 1041

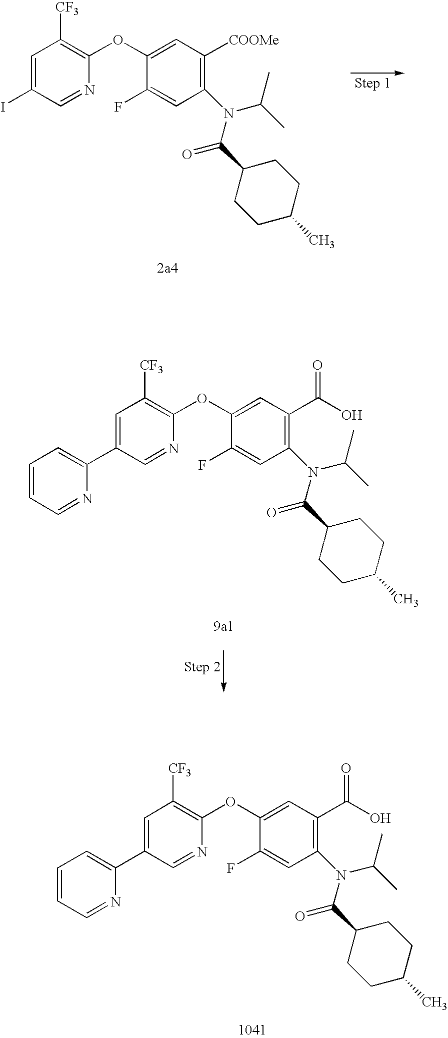

### Step 1:

To a mixture of iodide 2a4 (45 mg, 0.07 mmol), 2-tributylstannylpyridine (66 mg, 0.18 mmol), (Ph₃P)₄Pd (21 mg, 0.02 mmol) and degassed DMF (2 mL) is added 2 M aqueous Na₂CO₃ (0.14 mL, 0.29 mmol). The mixture is heated to 100° C. and is stirred overnight. The mixture is then allowed to cool before being diluted in EtOAc and washed with water and brine. The organic phase is then dried with MgSO₄, filtered and concentrated under reduced pressure. Purification by flash chromatography (20% EtOAc in Hex) affords biheteroaryl 9a1.

### Step 2:

To a mixture of ester 9a1 (20 mg, 0.03 mmol) with DMSO (2 mL), water (0.5 mL) and MeOH (1 mL) is added aqueous NaOH (10 N, 75 μL, 0.75 mmol). The mixture is stirred for about 1 hour at 50° C. before being acidified with TFA. The mixture is filtered and injected onto a preparative HPLC to isolate compound 1041.

### Example 10

### Preparation of Compound 1042

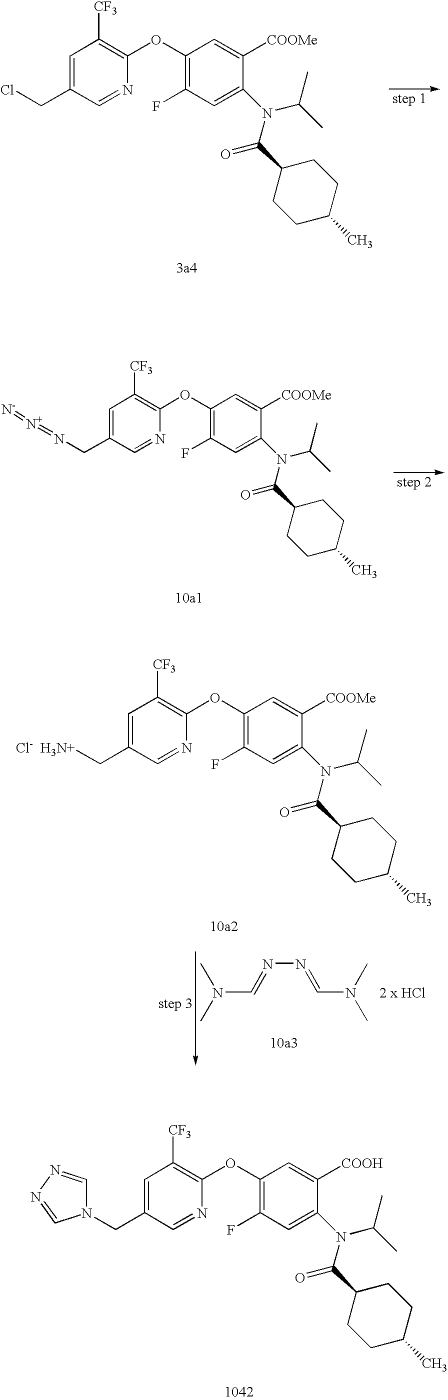

### Step 1:

Benzylchloride 3a4 (1.00 g, 1.8 mmol) is combined with NaN₃ (143 mg, 2.2 mmol) and KI (30 mg, 0.18 mmol) in anhydrous DMSO (15 mL). The mixture is heated to 65° C. and is stirred for about 1 hour. The mixture is diluted in EtOAc and washed with water and brine. The organic phase is dried with MgSO₄, filtered and concentrated under reduced pressure to provide azide 10a1 which is utilized without further purification.

### Step 2:

Azide 10a1 (0.95 g, 1.7 mmol) is combined with 10% Pd/C (95 mg) in MeOH (25 mL). The mixture is purged with H₂ then is stirred at RT overnight under 1 atm of H₂. The mixture is filtered through celite and then concentrated under reduced pressure. The residue is diluted in ether and then treated with HCl (1.0 N in ether, 10 mL). Solvent is removed in vacuo to afford HCl salt 10a2.

### Step 3:

Reference 1: Bartlett, R. K.; Humphrey, I. R. J. Chem. Soc. (C) 1967, 1664.

Reference 2: Robins M. J. J. Org. Chem. 2001, 66, 8204.

Amine hydrochloride salt 10a2 (75 mg, 0.13 mmol) is combined with azine 10a3 (114 mg, 0.53 mmol, prepared according to ref. 1) in anhydrous pyridine (2 mL). Chlorotrimethylsilane (85 μL, 0.66 mmol) is added and the mixture is heated to 100° C. and is stirred overnight. After the mixture cools to RT, DMSO (1 mL), MeOH, (1 mL) and water (0.5 mL) are added followed by NaOH (10 N, 200 μL, 2.0 mmol). The mixture is stirred overnight before being acidified with TFA, is partially concentrated and then injected onto the preparative HPLC to isolate 1042.

### Example 11A

### Method D

### Preparation of Compound 1062

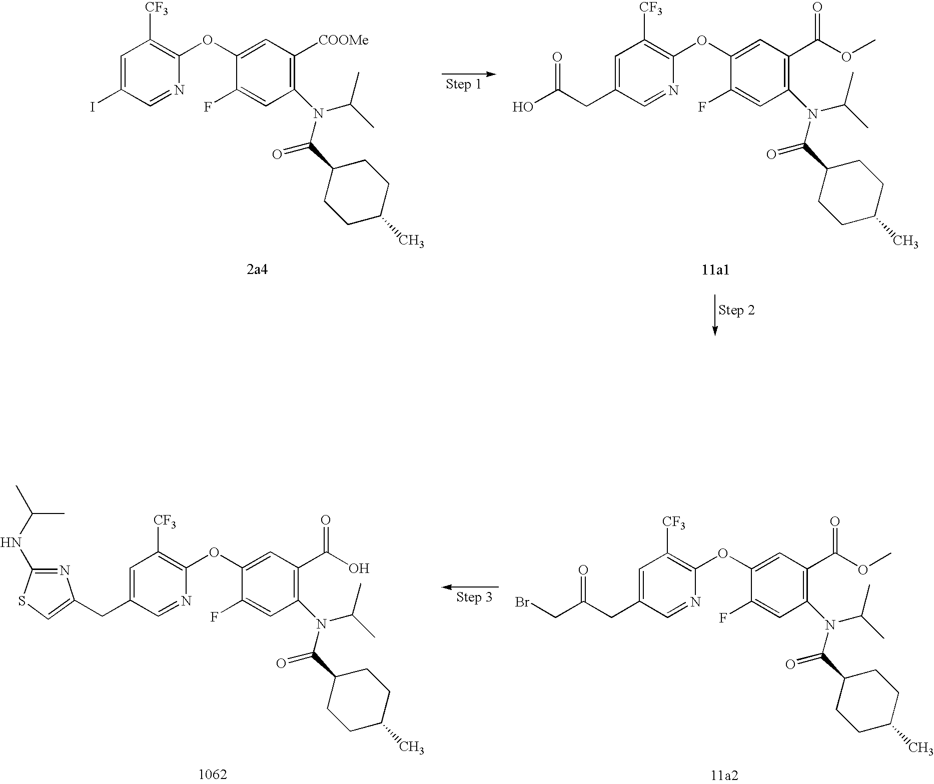

### Step 1:

Reference: Hennessy, E. J.; Buchwald, S. L. Org. Lett. 2002, 4, 269.

Iodide 2a4 (1.00 g, 1.6 mmol) is combined with dibenzylmalonate (1.8 mL, 7.2 mmol), CuI (109 mg, 0.57 mmol), 2-phenylphenol (97 mg, 0.57 mmol) and cesium carbonate (1.99 g, 6.1 mmol) in anhydrous THF (15 mL) and the mixture is degassed with Ar for 15 minutes. The reaction mixture is sealed and heated to 75° C. and is stirred for 16 h. Another portion of CuI (109 mg) and 2-phenylphenol (97 mg) are added and heating is continued for an additional 20 hours. The reaction mixture is taken-up in EtOAc and the solution is washed with NH₄Cl and brine. The organic phase is then dried with MgSO₄, filtered and concentrated under reduced pressure. The residue is diluted in EtOH (25 mL) and 10% Pd/C (175 mg) is added. Hydrogen is bubbled through the mixture for 10 minutes and then the mixture is stirred overnight under 1 atm of H₂. The reaction mixture is then filtered and concentrated in vacuo. Purification by flash chromatography (1:1 EtOAc/Hex) affords acid 11a1.

### Step 2:

To a mixture of acid 11a1 (105 mg, 0.19 mmol) and DMF (15 μL) in DCM (5 mL) is added (COCl)₂ (2.0 M in DCM, 140 μL, 0.28 mmol). The mixture is stirred for about 1 hour at RT before being concentrated in vacuo. DCM is added to the residue and the mixture is treated with CH₂N₂ (0.35 M solution in ether, 1.6 mL, 1.1 mmol) and then is stirred for 1 hour at RT. The mixture is concentrated in vacuo once again and THF (5 mL) is added. The mixture is chilled to 0° C. and aqueous HBr (48% solution, 200 μL) is added. After stirring for 20 minutes, the mixture is diluted in EtOAc and washed with water, saturated aqueous NaHCO₃ and brine. The organic phase is then dried with MgSO₄, filtered and concentrated under reduced pressure. Bromoketone 11a2 is utilized without further purification.

### Step 3:

Bromoketone 11a2 (40 mg, 0.06 mmol) is combined with isopropylthiourea (8 mg, 0.07 mmol) in i-PrOH (1 mL). The mixture is heated to 80° C. and is stirred for 1 hour before being cooled to RT and 2.5 N NaOH (150 μL, 0.38 mmol) is added. The mixture is stirred for about 4 hours at RT before being acidified with AcOH and injected onto the preparative HPLC to isolate 1062.

### Example 12A

### Method E

### Preparation of Compound 1044

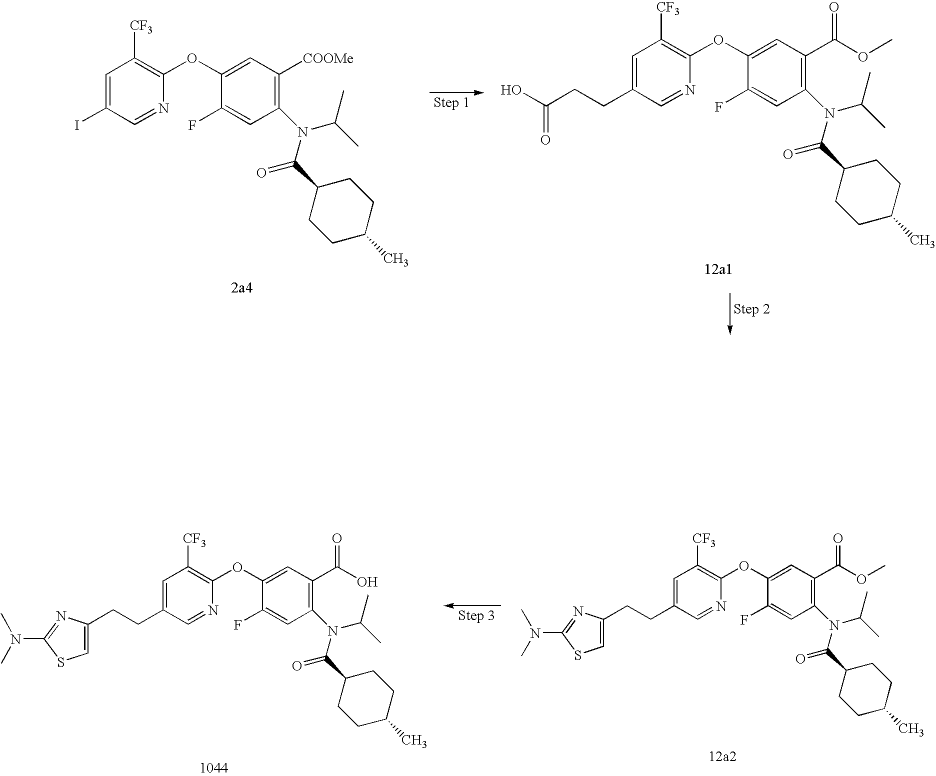

### Step 1a:

Iodide 2a4 (520 mg, 0.84 mmol) is combined with benzylacrylate (1.50 g, 9.3 mmol), triethylamine (5 mL) and Pd(OAc)₂ (50 mg, 0.22 mmol) in MeCN (20 mL). The vessel is sealed, heated to 60° C. and is stirred for 6 hours. The mixture is concentrated under reduced pressure and then the residue is subjected to flash chromatography (30 to 50% EtOAc in Hex) to afford the benzyl acrylate intermediate.

### Step 1b:

The benzylacrylate intermediate is combined with EtOH (20 mL) and 10% Pd/C (50 mg). The vessel is purged with H₂ and the mixture is stirred under 1 atm of H₂ for about 30 minutes. The mixture is filtered through a pad of celite then concentrated in vacuo to provide acid 12a1.

### Step 2a:

To a mixture of acid 12a1 (495 mg, 0.87 mmol) and DMF (30 μL) in DCM (20 mL) is added (COCl)₂ (2.0 M in DCM, 1.04 mL, 2.1 mmol). The mixture is stirred for about 1 hour at RT before being concentrated in vacuo. DCM (10 mL) is added to the residue and the mixture is treated with CH₂N₂ (0.9 M solution in ether, 5.7 mL, 5.0 mmol) then is stirred for about 30 minutes at RT. The mixture is concentrated in vacuo once again and THF (8 mL) is added. The mixture is chilled to 0° C. and aqueous HBr (48% solution, 0.4 mL) is added. After stirring for 20 minutes, the mixture is diluted in EtOAc and washed with water, saturated aqueous NaHCO₃ and brine. The organic phase is then dried with MgSO₄, filtered and concentrated under reduced pressure to afford the crude bromoketone intermediate.

### Step 2b:

The bromoketone intermediate is combined with 1,1-dimethylthiourea (187 mg, 1.8 mmol) in i-PrOH (15 mL). The mixture is heated to 80° C. and is stirred for about 1 hour. The reaction mixture is concentrated in vacuo and the resulting residue is subjected to flash chromatography to afford thiazole 12a2.

### Step 3:

Saponification under the conditions described in example 9 step 2 convert ester 12a2 to compound 1044.

### Example 13A

### Preparation of Compound 1045

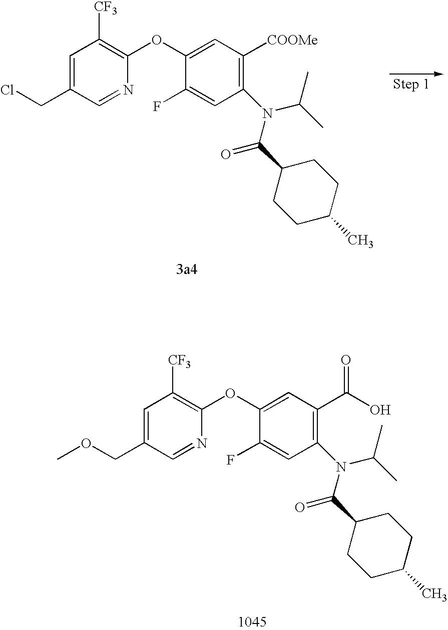

### Step 1:

Sodium methoxide (25% in MeOH, 17 μL, 0.08 mmol) is added to a mixture of benzyl chloride 3a4 (40 mg, 0.07 mmol) in MeOH (5 mL) then is stirred at ambient temperature for 16 hours. DMSO (1 mL) is added to the mixture followed by NaOH (2.5 N, 240 μL, 0.6 mmol), the resulting mixture is then stirred for 1 hour at ambient temperature. The mixture is acidified with AcOH, concentrated to 2 mL under reduced pressure, then injected into a preparative HPLC to isolated compound 1045.

### Example 14A

### Method F

### Preparation of Compound 1046

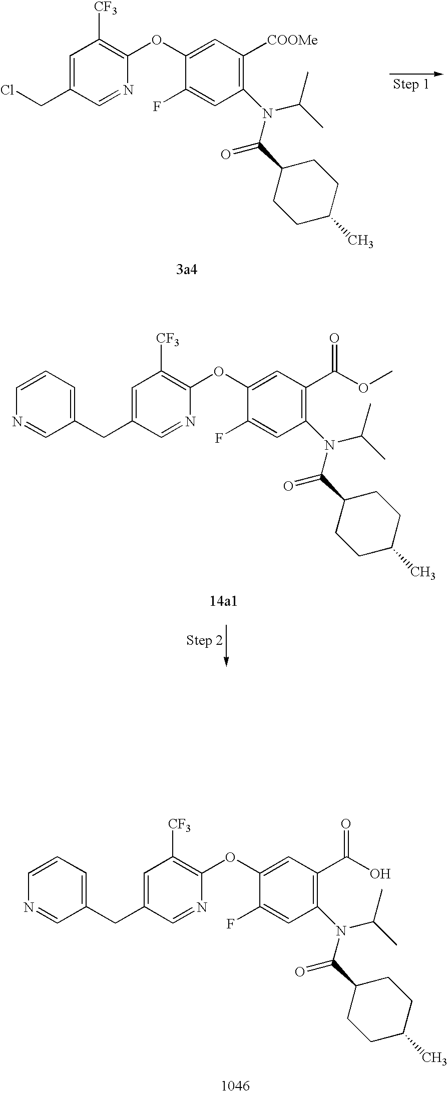

### Step 1:

Protocol adapted from: Nobre, S. M.; Monteiro, A. L. Tet. Lett. 2004, 45, 8225.

Triphenylphosphine (10 mg, 0.04 mmol), Pd(OAc)₂ (4.5 mg, 0.02 mmol), powdered K₃PO₄ (81 mg, 0.38 mmol), 3-pyridylboronic acid (35 mg, 0.28 mmol) and benzylchloride 3a4 (50 mg, 0.09 mmol) are combined in degassed (N₂) DMF (2.5 mL). The mixture is heated with stirring at 120° C. for 15 minutes in a microwave oven. The mixture is diluted in EtOAc (50 ml) then washed with 10% aqueous citric acid, water, saturated aqueous NaHCO₃ and brine. The organic phase is dried with MgSO₄ then filtered. Silica gel is added to the solution then the solvent is removed under reduced pressure. The silica gel dry packed compound is purified by combiflash (40 to 100% EtOAc/Hex gradient) to isolate compound 14a1.

### Step 2:

To a mixture of the ester 14a1 (33 mg, 0.06 mmol) dissolved in THF (3 mL)/MeOH (1 mL)/water (0.3 mL) is added NaOH (10 N, 200 μL, 2.0 mmol). The mixture is stirred at ambient temperature overnight. The mixture is carefully concentrated then partitioned between ether/hex (10 ml) and saturated NaHCO₃ (5 mL). The aqueous layer is extracted with ether. The aqueous layer is separated, acidified with TFA, then extracted with EtOAc (50 ml). The organic extract is washed with water and brine, dried with MgSO₄ then filtered. The solvent is removed under reduced pressure to provide compound 1046.

### Example 15A

### Method G

### Preparation of Compound 1051

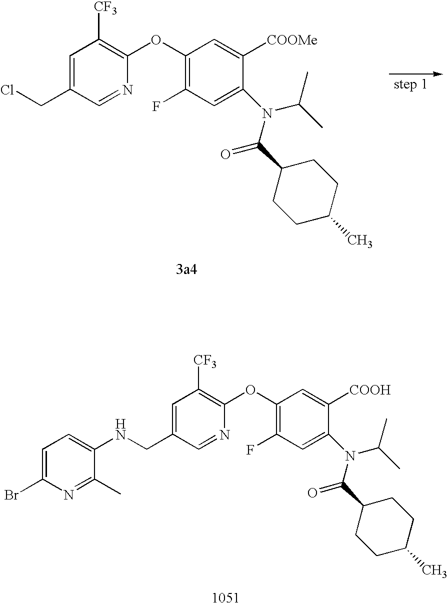

### Step 1:

Benzylchloride 3a4 (50 mg, 0.09 mmol) is combined with 2-methyl-3-amino-6-bromopyridine (30 mg, 1.6 mmol) and Et₃N (30 μL, 0.18 mmol) in DMF (1 mL). The mixture is heated to 110° C. and is stirred for 2 days. Tetrahydrofuran (2 mL), MeOH (1 mL) are added followed by aqueous NaOH (1 N, 2 mL, 2.0 mmol) then the mixture is further stirred at RT for about 14 hours. The mixture is acidified with AcOH and purified by preparative HPLC to isolate compound 1051.

### Example 16A

### Preparation of Compound 1054

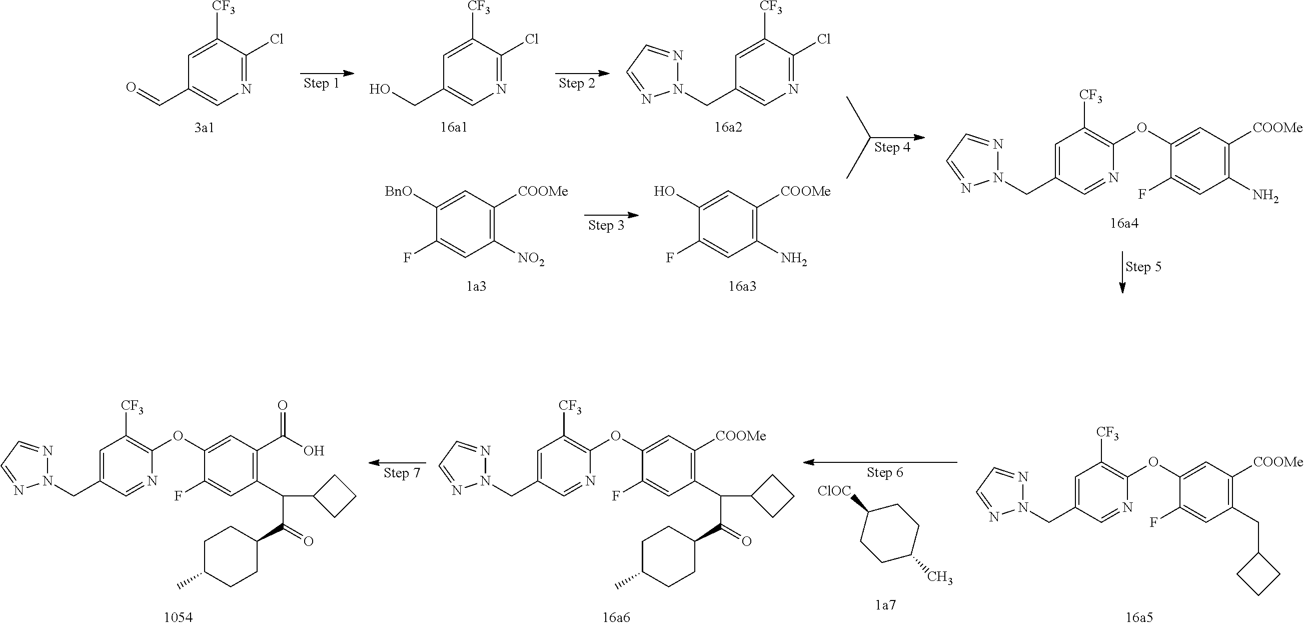

### Step 1:

A mixture of aldehyde 3a1 (19 g, 81 mmol) in methanol (225 mL) is chilled to 0° C. Sodium borohydride (4.1 g, 109 mmol) is added portion-wise and the mixture is stirred at 0° C. for 1.5 hours. Another portion of NaBH₄ (1 g) is added and the mixture is stirred another 30 minutes. The reaction is quenched by the addition of NaHSO₄ (5% aqueous) then diluted in EtOAc (500 mL). The organic phase is separated then washed with water (500 mL) and brine. The organic phase is dried over Na₂SO₄, filtered then concentrated under reduced pressure. The residue is subjected to flash chromatography (1:1 EtOAc/Hex) to isolate alcohol 16a1.

### Step 2:

Alcohol 16a1 (10.5 g, 48 mmol) is combined with triazole (3.42 g, 48 mmol) and triphenylphosphine (14.3 g, 54 mmol) in anhydrous THF (500 mL). The mixture is chilled to 0° C. and DIAD (10.6 mL, 54 mmol) is added drop-wise. Stirring is continued at 0° C. for about 1 hour before the mixture is allowed to warm to ambient temperature. The mixture is then stirred overnight. The mixture is diluted in EtOAc and washed with water (500 mL) and brine (500 mL) before being dried of Na₂SO₄. The solvents are removed under reduced pressure and the residue is subjected to flash chromatography (1:3 EtOAc/Hex) to afford benzylic triazole 16a2.

### Step 3:

Benzylether 1a3 (56.7 g, 186 mmol) is combined with MeOH (300 mL) and EtOAc (300 mL) in a Parr™ Bomb. The solution is degassed with Ar then Pearlman's catalyst (6 g) is added and the bomb charged with 30 psi of H₂ and is stirred at RT overnight. The mixture is filtered and the solvent is removed in vacuo. The residue is triturated with hexane to afford phenol 16a3.

### Step 4:

The S_{N}Ar coupling of phenol 16a3 with chloropyridine 16a2 to produce intermediate 16a4 is performed as described in example 2 step 3.

### Step 5:

Reference: Apodacca, R.; Xiao, W. Org. Lett. 2001, 3, 1745

To a mixture of aniline 16a4 (52 mg, 0.13 mmol) in THF (1.5 mL) is added cyclobutanone (19 μL, 0.25 mmol) followed by Bu₂SnCl₂ (2 mg, 0.01 mmol). The mixture is stirred for 5 minutes at ambient temperature before phenylsilane (17 μL, 0.14 mmol) is added. The mixture is heated to 70° C. and is then stirred for 4 hours before the mixture is diluted with saturated aqueous NaHCO₃ then extracted with EtOAc (×3). The combined organic extracts are washed with brine then dried over MgSO₄, filtered and concentrated. The residue is subjected to flash chromatography to provide cyclobutylaniline 16a5.

### Step 6:

To a mixture of cyclobutylaniline 16a5 (43 mg, 0.10 mmol) in anhydrous DCE (1.5 mL) is added acid chloride 1a7 (90 mg, 0.56 mmol), DMAP (18 mg, 0.15 mmol) and anhydrous pyridine (40 μL, 1.2 mmol). The mixture is heated in a microwave oven at 175° C. for about 15 minutes. The mixture is diluted with saturated aqueous NaHCO₃ then extracted with EtOAc (×2). The combined organic extracts are washed with brine then dried over MgSO₄, filtered and concentrated. Crude 16a6 is utilized in the next step without further purification.

### Step 7:

Ester 16a6 (45 mg, 0.08 mmol) is combined with THF (1 mL) and MeOH (0.5 mL) and water (0.5 mL). Sodium hydroxide (10 N, 76 μL, 0.76 mmol) is added and the mixture is stirred at RT overnight. The mixture is acidified with AcOH (83 μL, 1.08 mmol) then concentrated. The residue is taken-up in MeCN and water and then injected onto a preparative HPLC for purification to isolate compound 1054.

### Example 17A

### Method H

### Preparation of Compound 1055

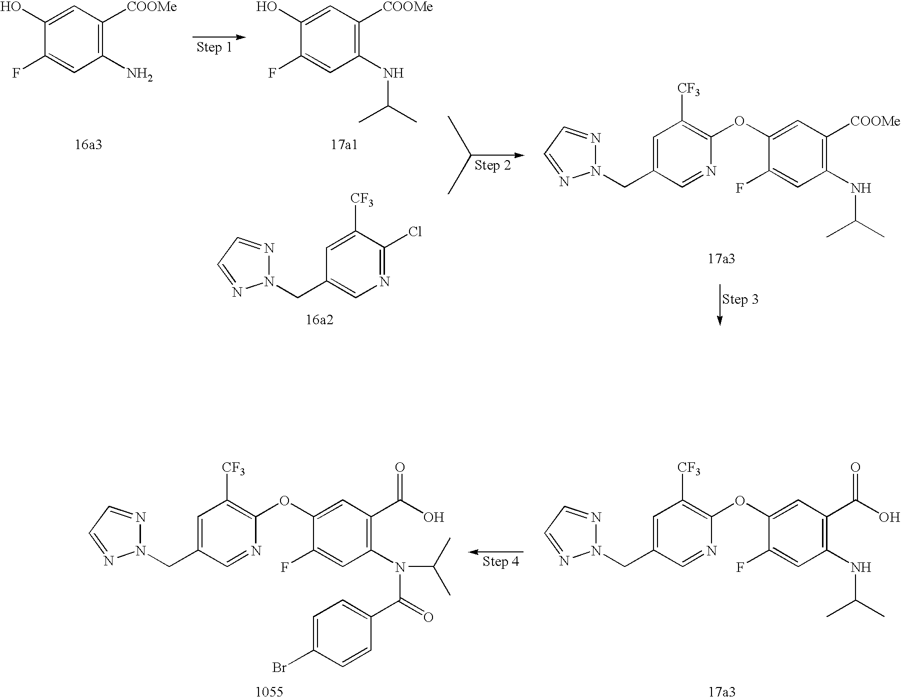

### Step 1:

To a mixture of aniline 16a3 (5.00 g, 27 mmol) and DCM (200 mL) is added HCl (1.0 M in ether, 27 mL, 27 mmol). After stirring for 5 minutes at ambient temperature, 2-methoxypropene (3.8 mL, 40 mmol) is added followed by sodium triacetoxyborohydride (11.4 g, 54 mmol) and the mixture is stirred for about 2 hours. The reaction mixture is diluted in EtOAc and washed with saturated aqueous NaHCO₃ and brine. The organic phase is dried with MgSO₄ then filtered. Silica gel is added to the solution then the solvent is removed under reduced pressure. The silica gel dry packed compound is purified by combiflash (5 to 30% EtOAc/Hex gradient) to isolate i-Pr-aniline 17a1.

### Step 2:

The S_{N}Ar coupling of phenol 17a1 with chloropyridine 16a2 to produce intermediate 17a2 is performed as described in example 2A step 3.

### Step 3:

Saponification of ester 17a2 to acid 17a3 is performed as described in example 16A step 7.

### Step 4:

To a mixture of anthranilic acid 17a3 (25 mg, 0.06 mmol) in anhydrous DCE (2 mL) is added 4-bromobenzoylchloride (18 mg, 0.08 mmol) and anhydrous pyridine (14 μL, 0.17 mmol). The mixture is heated in a microwave oven at 125° C. for about 20 minutes. The mixture is acidified with TFA then injected onto a preparative HPLC to isolate compound 1055.

### Example 18A

### Preparation of Compound 1057

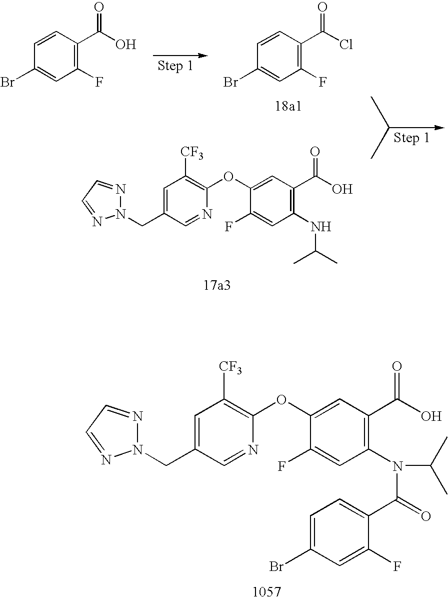

### Step 1:

To a mixture of 4-bromo-2-fluorobenzoic acid (75 mg, 0.34 mmol) and DMF (5 μL) in DCM (2 mL) is added oxalyl chloride (30 μL, 0.34 mmol). The mixture is stirred for about 1 hour at RT then is concentrated in vacuo to afford crude acid chloride 18a1 which is utilized without further purification.

### Step 2:

Coupling of acid chloride 18a1 to anthranilic acid 17a3 is performed as described in example 17A step 4.

### Example 19A

### Method I

### Preparation of Compound 1058

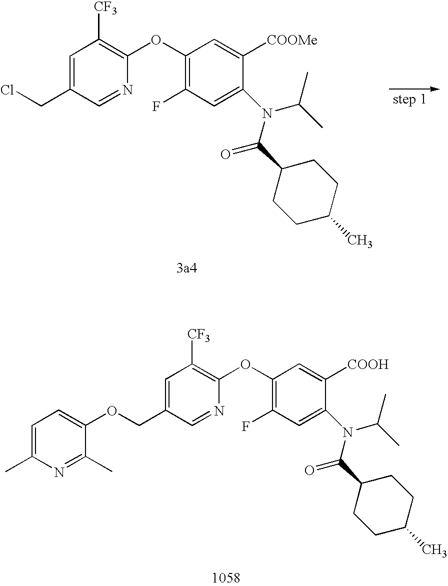

### Step 1:

To a mixture of 3-hydroxy-2,6-dimethylpyridine (20 mg, 0.17 mmol) in DMF (1.4 mL) is added NaH (95%, 5 mg, 0.21 mmol). The mixture is stirred for about 15 minutes before benzylchloride 3a4 (75 mg, 0.14 mmol) is added. The mixture is stirred at RT overnight. Methanol (0.5 mL) and LiOH (60 mg, 1.4 mmol) are added and the mixture is further stirred at RT overnight. The mixture is acidified with AcOH and purified by preparative HPLC to isolate compound 1058.

### Example 20

### Preparation of Compound 1059

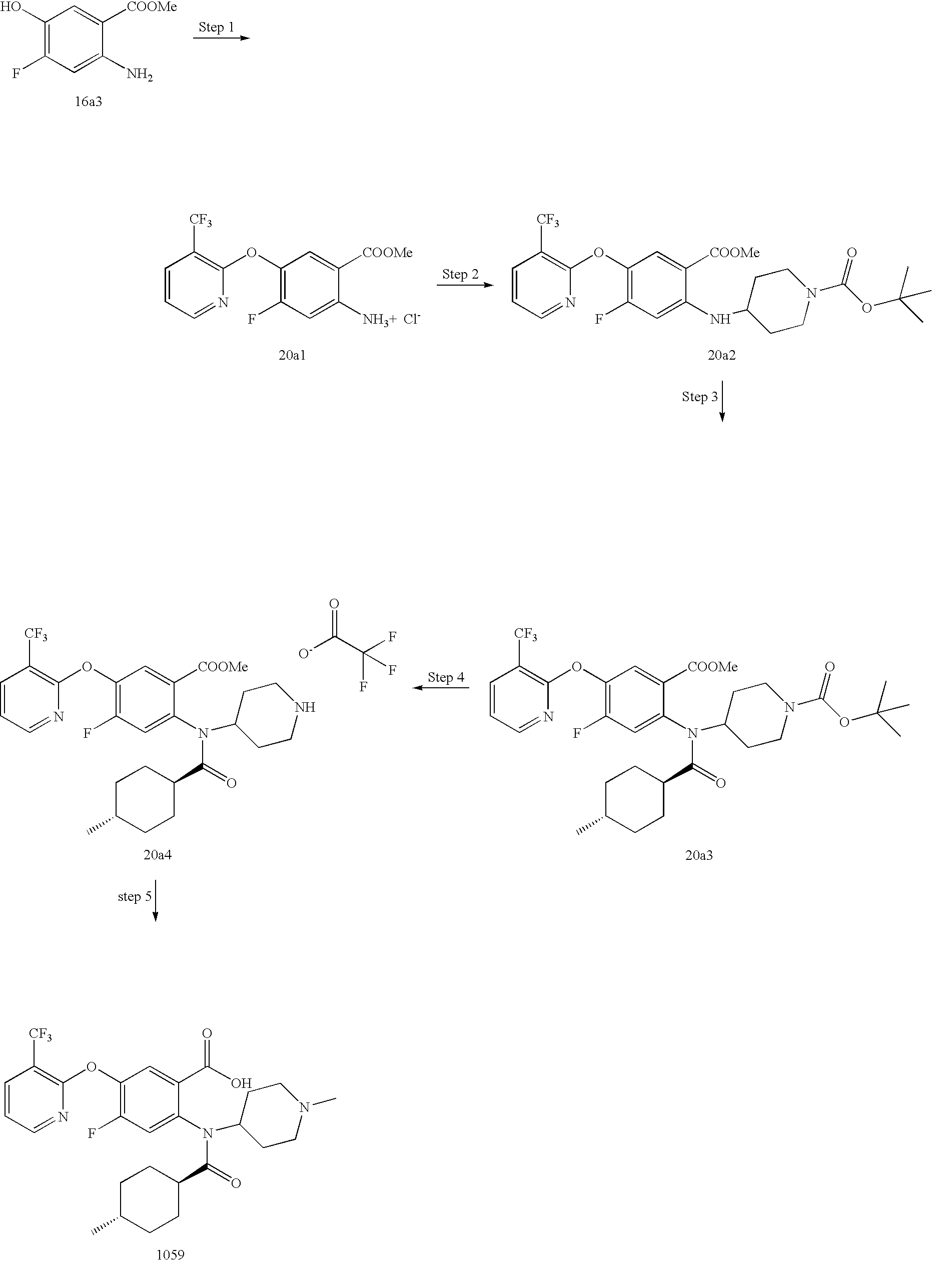

### Step 1:

To a mixture of phenol 16a3 (740 mg, 4.0 mmol) and 2-fluoro-3-trifluoromethylpyridine (990 mg, 6.0 mmol) in anhydrous DMSO (8 mL) is added powdered potassium carbonate (1.7 g, 12 mmol). The mixture is stirred at 90° C. for about 2 hours. The mixture is allowed to cool to ambient temperature then is taken in EtOAc (50 mL) and washed with 10% aqueous citric acid, water, saturated aqueous NaHCO₃ and brine. The organic phase is dried with MgSO₄ and filtered, then concentrated under reduced pressure. The crude residue is diluted in EtOAc and HCl (1 N solution in ether, 5 mL, 5.0 mmol) is added. Solid HCl salt 20a1 is collected by filtration and washed with ether/hexanes (1:2 mixture).

### Step 2:

The reductive amination procedure described in example 16A step 5 is used to convert aniline 20a1 to 20a2 using

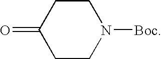

### Step 3:

Compound 20a2 is first saponified then the aniline is acylated with acid chloride 1a7 under conditions described in example 17A steps 3 & 4. The resulting carboxylic acid is treated with diazomethane in ether to recover ester 20a3.

### Step 4:

To a mixture of Boc-piperidine 20a3 (119 mg, 0.19 mmol) in DCM (2 mL) is added TFA (2.5 mL). The mixture is stirred for about 2 hours at ambient temperature then is concentrated under reduced pressure. Crude TFA salt 20a4 is utilized in the next step without further purification.

### Step 5:

To a mixture of piperidine TFA salt 20a4 (60 mg, 0.09 mmol) in EtOH (2 mL) is added formaldehyde (37% aqueous, 42 μL, 0.52 mmol), sodium cyanoborohydride (35 mg, 0.55 mmol) and AcOH (100 μL). The mixture is stirred at ambient temperature overnight. DMSO (2 mL) is added to the mixture followed by aqueous 5 N LiOH (0.5 mL, 2.5 mmol). The mixture is stirred at ambient temperature overnight. The mixture is acidified with TFA then injected onto a preparative HPLC to isolate compound 1059.

### Example 21A

### Preparation of Compound 1060

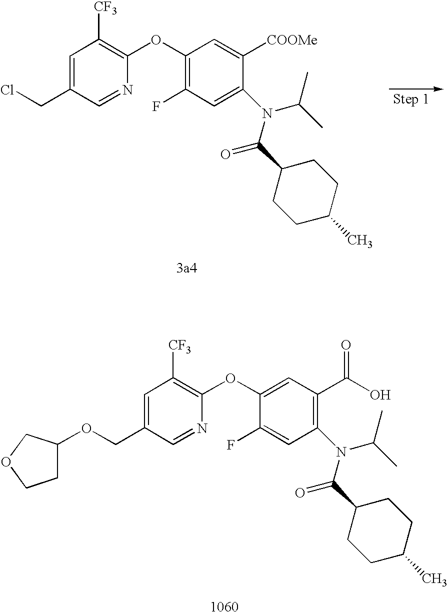

### Step 1:

To a mixture of 3-hydroxytetrahydrofuran (17 mg, 0.19 mmol) in DMF (1 mL) is added NaH (95%, 5 mg, 0.22 mmol). The mixture is stirred for 15 minutes before benzylchloride 3a4 (35 mg, 0.06 mmol) in DMF (1 mL) is added. The mixture is stirred at RT overnight. Methanol (0.5 mL), water (0.3 mL) and NaOH (10 N, 0.3 mL, 3.0 mmol) are added and the mixture is further stirred at RT for about 2.5 hours. The mixture is acidified with TFA, partially concentrated, diluted with DMSO (1 mL), then purified by preparative HPLC to isolate compound 1060.

### Example 22A

### Preparation of Compound 1070

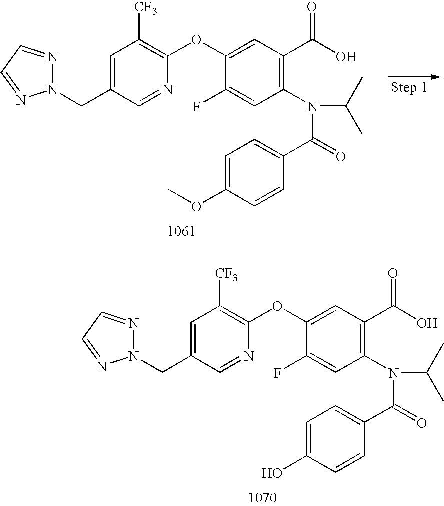

### Step 1:

To compound 1061 (prepared by Method H) (50 mg, 0.09 mmol) in anhydrous DCM (2 mL) is added BBr₃ (1.0 M solution in DCM, 435 μL, 0.43 mL). The mixture is stirred at ambient temperature for 2 hours. The mixture is concentrated under reduced pressure, then the residue is diluted in DMSO and injected onto a preparative HPLC to isolate compound 1070.

### Example 23A

### Method J

### Preparation of Compound 1082

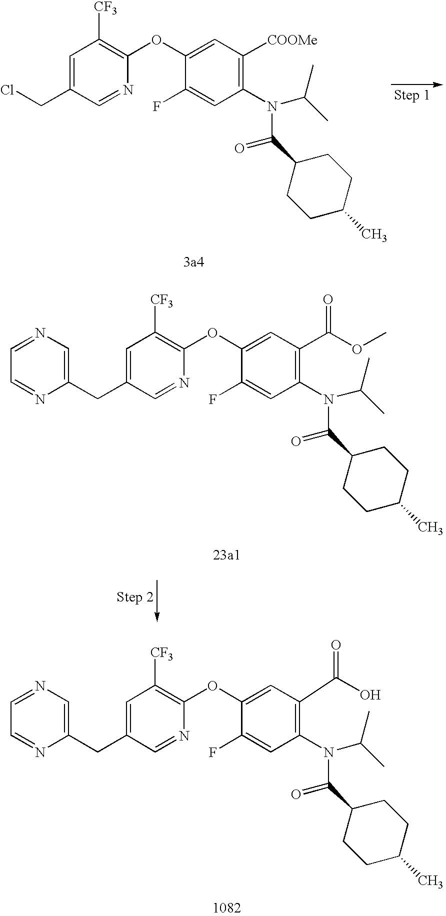

### Step 1:

To a degassed (N₂) mixture of benzyl chloride 3a4 (527 mg, 1.0 mmol) in anhydrous DMF is added 2-tributylstannylpyrazine (738 mg, 2.0 mmol) and tetrakis(triphenylphosphino) palladium (0) (116 mg, 0.1 mmol). The mixture is heated in a microwave at 120° C. for 20 minutes. The mixture is diluted in EtOAc/Ether (100/50 ml) and washed with water and brine. The organic phase is dried with MgSO₄, filtered, the solvent is removed under reduced pressure. The residue is subjected to flash chromatography (15% i-PrOH/Hex then 2:1 EtOAc/Hex) to isolate 23a1.

### Step 2:

To a mixture of ester 23a1 (435 mg, 0.76 mmol) in THF (10 mL), MeOH (2 mL) and water (1 mL) is added NaOH (10N, 533 μL, 5.3 mmol). The mixture is stirred overnight at ambient temperature. More NaOH is added (10N, 354 μL, 3.5 mmol) and stirring is continued for about 6 hours. The mixture is partitioned between water (25 mL) and ether (50 mL). The aqueous phase is diluted with 0.5 N KHSO₄ then extracted with EtOAc (125 mL). The organic phase is then washed with water and brine. The organic phase is dried with MgSO₄, filtered and the solvent is removed under reduced pressure. The residue is subjected to preparative HPLC to isolate compound 1082.

### Example 23A

### Method K

### Preparation of Compound 1079

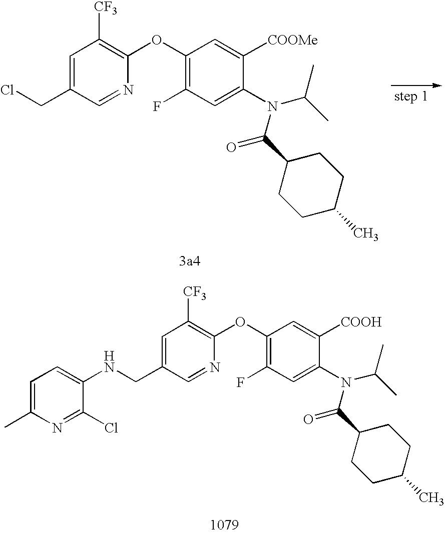

### Step 1:

Benzylchloride 3a4 (50 mg, 0.09 mmol) is combined with 3-amino-2-chloro-6-methylpyridine (20 mg, 1.4 mmol) and DIPEA (30 μL, 0.18 mmol) in DMF (1 mL). The mixture is heated to reflux and is stirred overnight. Tetrahydrofuran (1 mL), and MeOH (1 mL) are added followed by aqueous NaOH (1 N, 1 mL, 2.0 mmol) then the mixture is stirred at RT for about 14 hours. The mixture is acidified with AcOH and purified by preparative HPLC to isolate compound 1079.

### Example 24A

### Preparation of Compound 1097

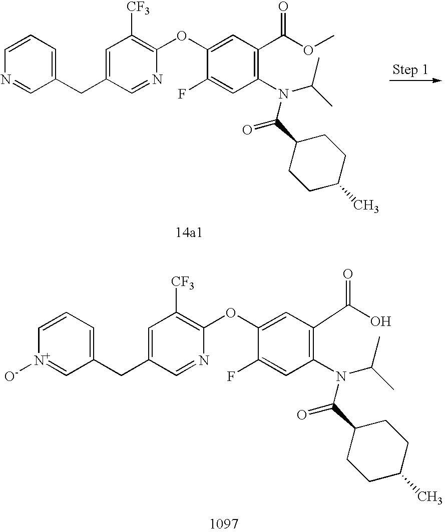

### Step 1:

To a mixture of ester 14a1 (83 mg, 0.14 mmol) and anhydrous DCM is added m-CPBA (50 mg, 0.25 mmol). The mixture is stirred at ambient temperature under N₂ overnight. The mixture is diluted in EtOAc (50 ml) then washed with water, 10% aqueous sodium thiosulphate, water, 1N NaOH, water and brine. The combined organic extracts are dried over MgSO₄, filtered and concentrated under reduced pressure. The residue is then diluted in DMSO (2 mL), MeOH (1 mL) and water (0.2 mL) before aqueous NaOH (10 N, 150 μL, 1.5 mmol) is added. The mixture is stirred at ambient temperature overnight before being acidified with TFA, concentrated then injected onto the preparative HPLC to isolate compound 1097.

### Example 25A

### Preparation of Compound 1101

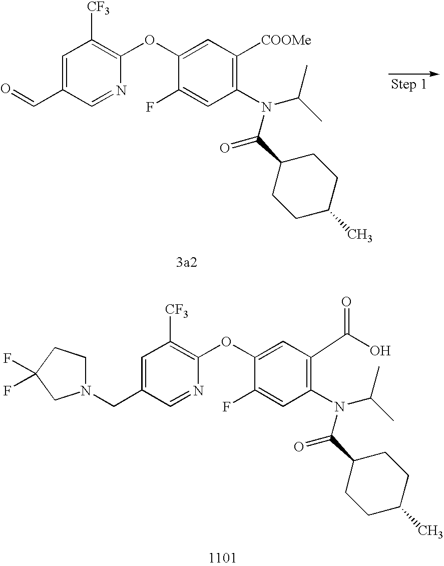

### Step 1:

Aldehyde 3a2 (31 mg, 0.06 mmol) is combined with 3,3-difluoropyrrolidine hydrochloride (13 mg, 0.09 mmol) in DCM (1 mL). To the mixture is added NaHB(OAc)₃ (14 mg, 0.23 mmol) and the mixture is stirred at RT overnight. The mixture is diluted in saturated aqueous NaHCO₃ then extracted with EtOAc (×3). The combined organic extracts are dried over MgSO₄, filtered and concentrated under reduced pressure. To the residue is added THF (1.5 mL), MeOH (0.75 mL) and water (0.75 mL) followed by NaOH (10 N, 60 μL, 0.6 mmol). The mixture is stirred for 3 days at RT before being acidified with AcOH, partially concentrated then injected onto the preparative HPLC to isolate compound 1101.

### Example 26A

### Preparation of Compound 1102

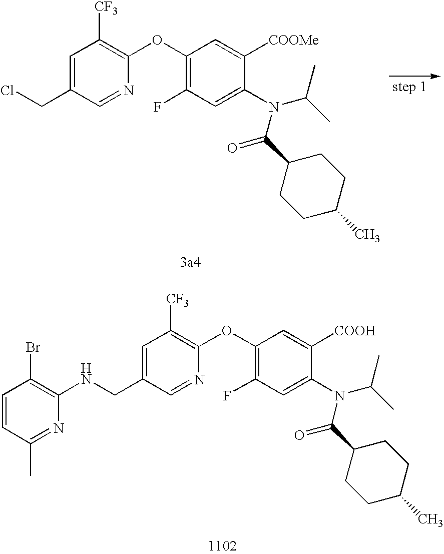

### Step 1:

Benzylchloride 3a4 (59 mg, 0.11 mmol) is combined with 2-amino-3-bromo-6-methylpyridine (30 mg, 0.16 mmol), TBABr (7 mg, 0.02 mmol) and DIPEA (40 μL, 0.22 mmol) in DMF (1 mL). The mixture is heated to 110° C. and is stirred for 1 day. Methanol (0.5 mL) is added followed by aqueous NaOH (1 N, 2 mL, 2.0 mmol) then the mixture is stirred at RT for 73 hours. The mixture is acidified with AcOH (2 mL) then the volatiles are removed in vacuo. The residue is taken-up in AcOH (2.5 mL) then injected onto the preparative HPLC to isolate compound 1102.

### Example 27A

### Preparation of Compound 1105

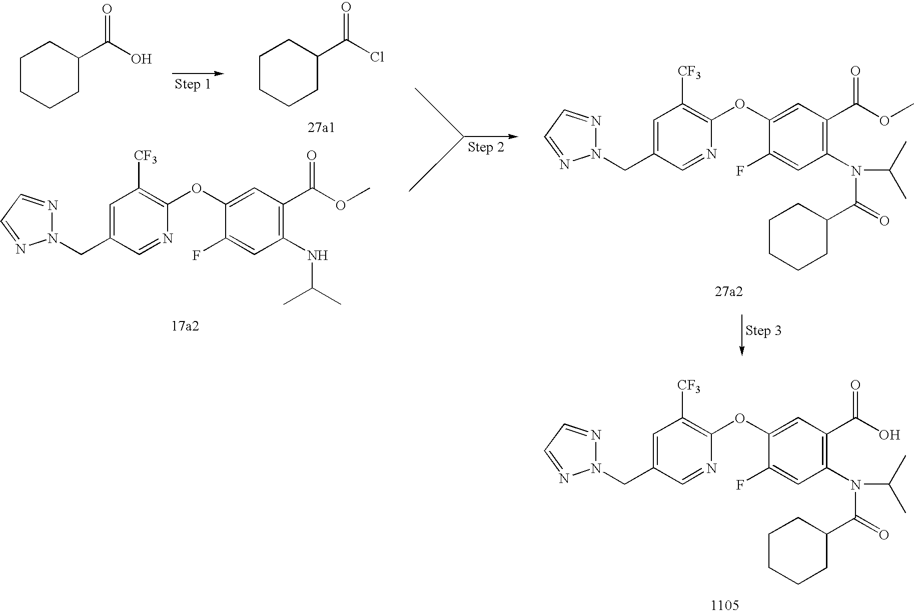

### Step 1:

Cyclohexane carbonyl chloride 27a1 is prepared from cyclohexane carboxylic acid as described in example 1A step 5.

### Step 2:

To a mixture of aniline 17a2 (80 mg, 0.18 mmol) in anhydrous DCE (2 mL) is added cyclohexane carbonyl chloride 27a1 (181 mg, 1.2 mmol) and anhydrous pyridine (171 μL, 2.1 mmol). The mixture is heated in a microwave oven at 170° C. for 30 minutes. The mixture is diluted in EtOAc then washed with saturated aqueous NaHCO₃ and brine. The organic phase is dried with Na₂SO₄ and filtered. Silica gel is added to the solution and then it is concentrated in vacuo. The silica gel dry packed compound is subjected to flash chromatography (30 to 90% EtOAc in Hex) to afford compound 27a2.

### Step 3:

To a mixture of ester 27a2 (43 mg, 0.08 mmol) in DMSO (0.5 mL) and THF (1.5 mL) is added NaOH (5 N, 153 μL, 0.76 mmol). The mixture is heated to 50° C. and is stirred for about 1 hour. Acetic acid (0.5 mL) and MeCN (1 mL) is added and the mixture is injected onto a preparative HPLC to isolate compound 1105.

### Example 28A

### Method L

### Preparation of Compound 1108

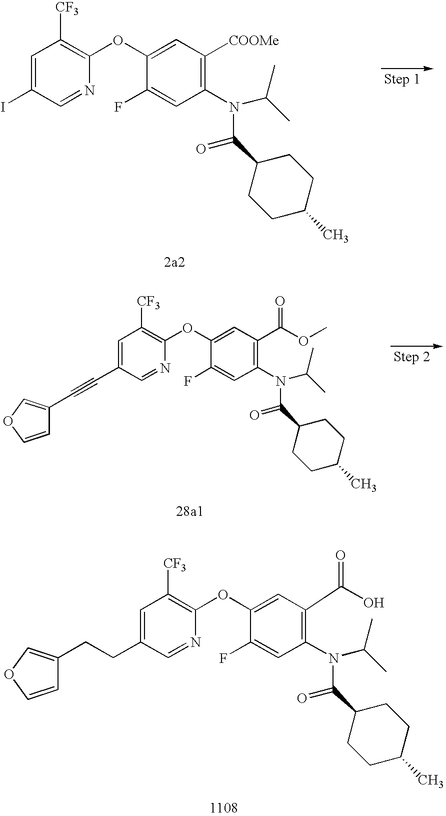

### Step 1:

Iodide 2a2 (65 mg, 0.10 mmol) is combined with furan-3-ylethynyltrimethylsilane (30 mg, 0.15 mmol), cuprous iodide (2 mg, 0.01 mmol), triethylamine (70 μL, 0.52 mmol), TBAF (1.0 M in THF, 110 μL, 0.11 mmol) and (PPh₃)₄Pd (12 mg, 0.01 mmol) in anhydrous DMF (1 mL). The mixture is heated in a microwave at 120° C. for 10 minutes. The crude reaction mixture is loaded directly onto a silica gel cartridge and purified on a combiflash to obtain alkyne 28a1.

### Step 2:

To a mixture of ester 28a1 (35 mg, 0.06 mmol) in EtOH (3 mL) is added 10% Pd/C (35 mg). Hydrogen is bubbled through the mixture for 5 minutes before the mixture is stirred for about 2 hours under 1 atm of H₂. The mixture is filtered through celite and concentrated. The crude product is saponified under conditions described in example 14A step 2 to afford compound 1108.

### Example 29A

### Preparation of Compound 1111

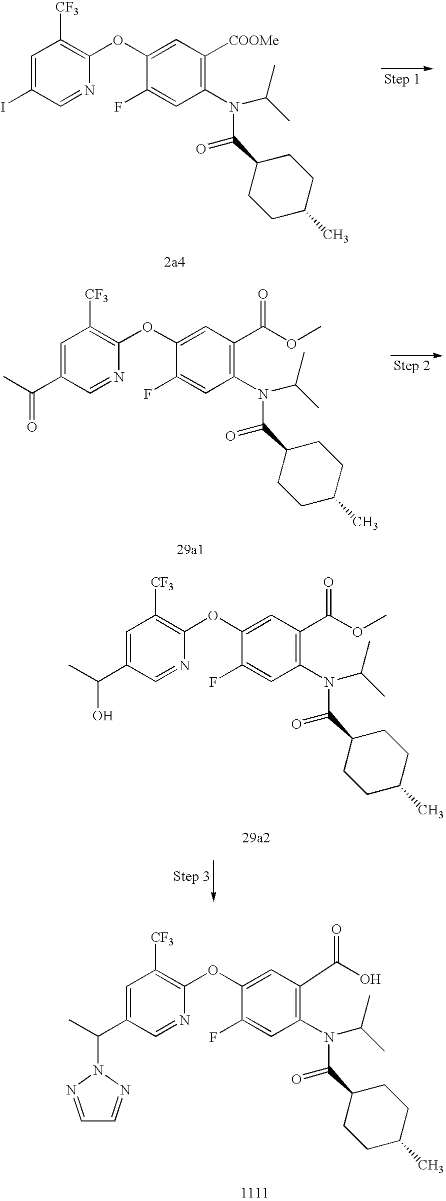

### Step 1:

Iodide 2a4 (338 mg, 0.54 mmol) is combined with ethylvinylether (520 μL, 5.4 mmol), Pd(OAc)₂ (12 mg, 0.05 mmol), PPh₃ (29 mg, 0.11 mmol) and K₂CO₃ (83 mg, 0.6 mmol) in DMF (2 mL). The mixture is heated to 200° C. in the microwave for 2 minutes. After the mixture cools to ambient temperature, HCl (4.0 M in dioxane, 1 mL) is added and the mixture is stirred for 1 hour at ambient temperature. The mixture is poured into saturated aqueous NaHCO₃ and extracted with DCM (×3). The combined organic extracts are washed with brine then dried over MgSO₄, filtered and concentrated under reduced pressure. Crude 29a1 is utilized without further purification.

### Step 2:

To a mixture of methylketone 29a1 (292 mg, 0.54 mol) in DCM (10 mL) is added NaBH₄ (103 mg, 2.7 mmol). The mixture is stirred at ambient temperature overnight. The mixture is poured into saturated aqueous NH₄Cl and extracted with DCM (×3). The combined organic extracts are washed with brine then dried over MgSO₄, filtered and concentrated under reduced pressure. Purification by flash chromatography (5 to 70% EtOAc in Hex) affords alcohol 29a2.

### Step 3:

A Mitsunobu reaction as described in example 16A step 2 followed by saponification as described in example 14A step 2 provides compound 1111.

### Example 30A

### Preparation of Compound 1114

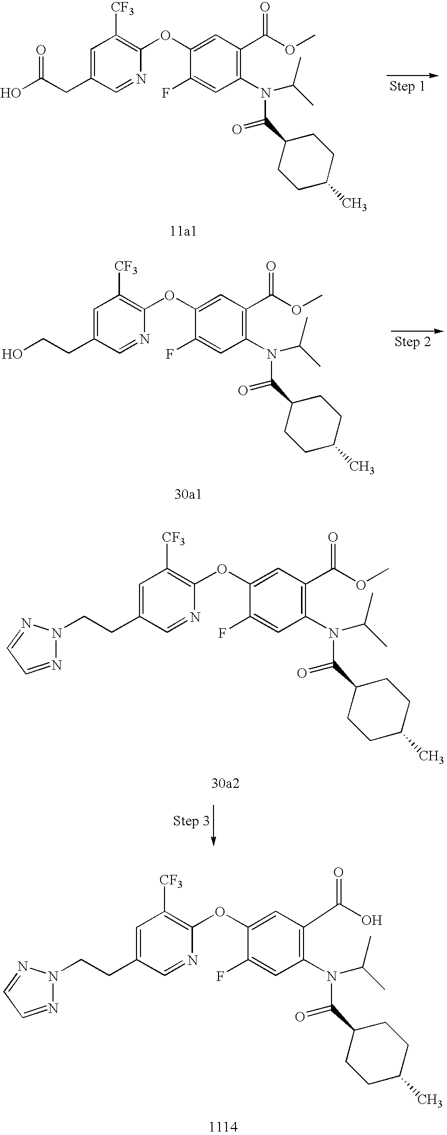

### Step 1:

To acid 11a1 (500 mg, 0.90 mmol) while stirring at 0° C. in THF (5 mL) is added BH₃-THF complex (1.0 M solution in THF, 2.25 mL, 2.25 mmol). The solution is allowed to warm to ambient temperature then is further stirred overnight. The mixture is quenched by pouring into water. The aqueous mixture is extracted with EtOAc (×2) then the combined organic extracts are washed with brine, dried over Na₂SO₄, filtered and concentrated under reduced pressure. Alcohol 30a1 is utilized without further purification.

### Step 2:

A Mitsunobu reaction as described in example 16A step 2 converts alcohol 30a1 to benzylic triazole 30a2.

### Step 3:

A saponification as performed in example 14A step 2 converts ester 30a2 to compound 1114.

### Example 31A

### Preparation of Compound 1115

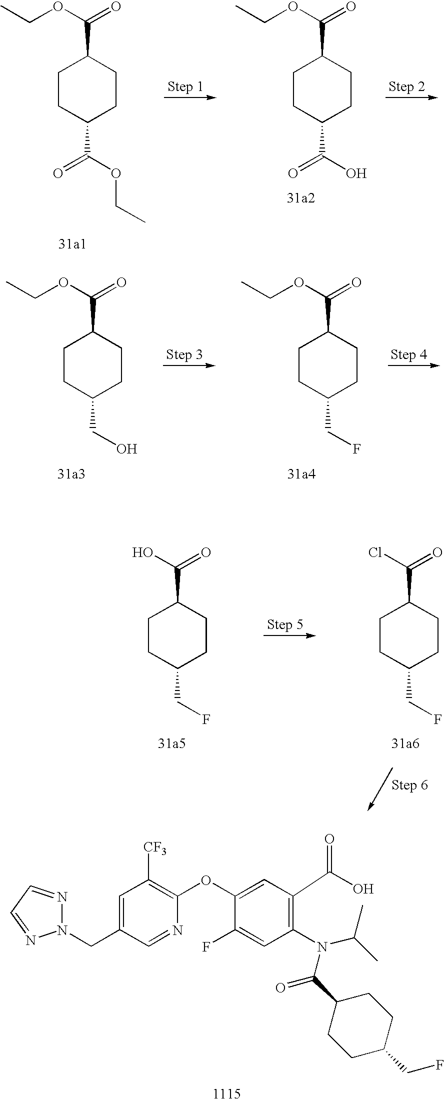

### Step 1:

To a mixture of dimethyl-trans-cyclohexanedicarboxylate 31a1 (30.0 g, 150 mmol) in MeOH (750 mL) is added NaOH (6.0 g, 150 mmol) in water (40 mL). The mixture is stirred at ambient temperature for 1 day before being partially concentrated under reduced pressure. After dilution in water, the mixture is extracted with EtOAc (×3) to separate unreacted 31a1. The pH of the aqueous phase is adjusted to 1 using 1 N HCl then extracted with EtOAc (×3). The combined organic extracts are dried over Na₂SO₄, filtered and concentrated under reduced pressure. Acid 31a2 is utilized without further purification.

### Step 2:

A mixture of acid 31a2 (24.7 g, 123 mmol), anhydrous THF (1.2 L) and NEt₃ (18.5 mL, 133 mmol) is chilled to −5° C. Ethylchloroformate (12.7 mL, 133 mmol) is added slowly maintaining the temperature between −5 and 0° C. After 1 hour, the mixture is filtered then added via cannula to a mixture of NaBH₄ (10.1 g, 266 mmol) in water (400 mL) at 10° C. The reaction is then quenched by adjusting to the pH of the mixture to 1 with 1 N HCl. The mixture is partitioned with EtOAc. The organic phase is dried over Na₂SO₄, filtered and concentrated under reduced pressure. Purification by flash chromatography (13 to 33% EtOAc in Hex) affords alcohol 31a3.

### Step 3:

To a mixture of alcohol 31a3 (5.67 g, 32.9 mmol) in DCM (300 mL) is added diethylaminosulfurtrifluoride (4.7 mL, 36 mmol). The mixture is stirred for 4 hours at RT before being filtered through a pad of silica gel (washed with 1:1 Hex/DCM). The filtered mixture is concentrated under reduced pressure then is subjected to flash chromatography to isolate fluoro-derivative 31a4.

### Step 4:

To a mixture of ester 31a4 (2.32 g, 13.3 mmol) in THF (60 mL) and water (50 mL) is added lithium hydroxide monohydrate (0.67 g, 16 mmol). The mixture is stirred for 6 hours at RT before the pH of the mixture is adjusted to 1 with 1 N HCl. The mixture is partitioned with EtOAc and the organic phase is separated then dried over Na₂SO₄, filtered and concentrated under reduced pressure. Purification by flash chromatography (25 to 50% EtOAc in Hex) affords acid 31a5.

### Step 5:

The acid 31a5 is converted to acid chloride 31a6 using the conditions described in example 1A step 5.

### Step 6:

The coupling of 31a6 and aniline 17a3 to produce 1115 is performed as shown in Method H.

### Example 32A

### Preparation of Compound 1116

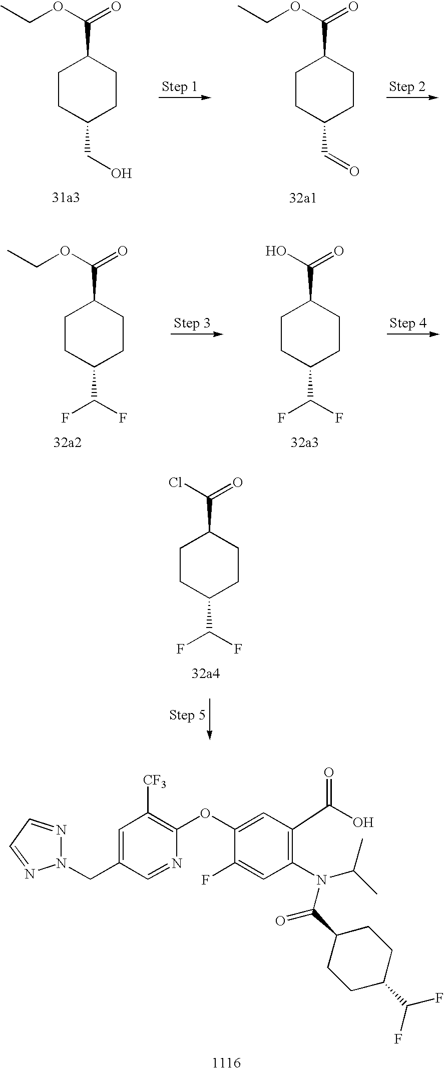

### Step 1:

The alcohol 31a3 (7.10 g, 41.2 mmol) is combined with NEt₃ (17.2 mL, 124 mmol) in DMSO (200 mL). Sulfur trioxide pyridine complex (16.40 g, 103.1 mmol, 2.5 eq) is added portion-wise and the resulting mixture is stirred at RT for 4 h. The reaction is quenched with water and the mixture is partitioned between EtOAc and water. The organic layer is dried over Na₂SO₄, filtered and concentrated under reduced pressure. The residue is subjected to flash chromatography separation (Hexanes:EtOAc 10:1 to 5:1) to isolate aldehyde 32a1.

### Step 2:

To a mixture of aldehyde 32a1 (4.00 g, 23.5 mmol) in DCM (110 mL) is added diethylaminosulfurtrifluoride (3.4 mL, 26 mmol). The mixture is stirred for about 5 hours at RT before being filtered through a pad of silica gel (washed with DCM). The filtered mixture is concentrated under reduced pressure then is subjected to flash chromatography (2:1 EtOAc/Hex) to isolate difluoro-derivative 32a2.

### Step 3:

Ester 32a2 is saponified under the conditions reported in example 31A step 4 to provide acid 32a3.

### Step 4:

The acid 32a3 is converted to acid chloride 32a4 using the conditions described in example 1A step 5.

### Step 5:

The coupling of 32a4 and aniline 17a3 to produce 1116 is performed as shown in Method H.

### Example 33A

### Method M

### Preparation of Compound 1117

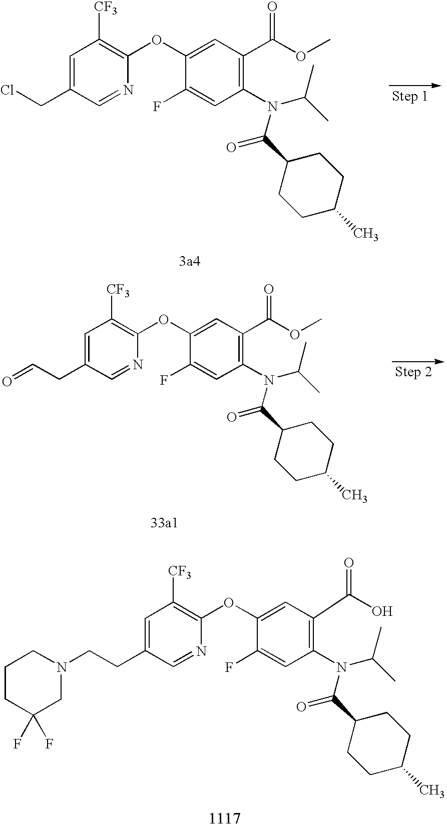

### Step 1:

Reference: Baillargeon, V. P.; Stille, J. K. J. Am. Chem. Soc. 1986, 108, 452.

To a mixture of benzylchloride 3a4 (1.0 g, 1.8 mmol) in THF (10 mL) is added (Ph₃P)₄Pd (636 mg, 0.55 mmol). The vessel is purged with CO. The mixture is warmed to 50° C. and, while CO is bubbled directly into the reaction mixture, Bu₃SnH (543 μL, 2.0 mmol) in THF (60 mL) is added over 2 hours using a syringe pump. Stirring is continued for another 18 hours at 50° C. The reaction mixture is concentrated under reduced pressure and the residue is subjected to flash chromatography (10 to 100% EtOAc/Hex) to afford aldehyde 33a1.

### Step 2:

To a mixture of aldehyde 33a1 (40 mg, 0.07 mmol) and 3,3-difluoropiperidine hydrochloride (47 mg, 0.30 mmol) in DCM (1 mL) is added NaHB(OAc)₃ (31 mg, 0.15 mmol). The mixture is stirred at RT for about 18 hours. Tetrahydrofuran (2 mL), MeOH (1 mL), NaOH (1 N, 1 mL, 1.0 mmol) and LiOH—H₂O (15 mg, 0.35 mmol) are added and the mixture is stirred for 3 hours. The mixture is concentrated, taken-up in AcOH (2.5 mL), filtered then injected onto a preparative HPLC to isolate compound 1117.

### Example 34A

### Preparation of Compound 1118

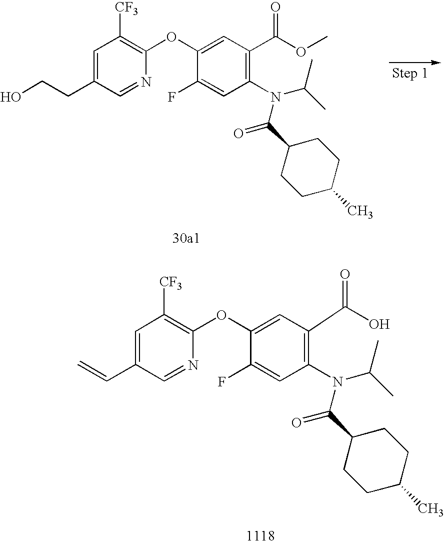

### Step 1:

Compound 1118 is isolated from alcohol 30a1 using the Mitsunobu conditions described in example 30A step 2.

### Example 35A

### Preparation of Compound 1119

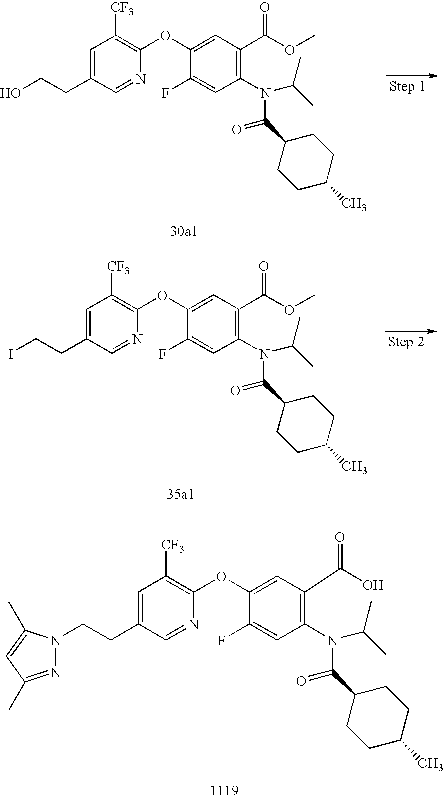

### Step 1:

Alcohol 30a1 (76 mg, 0.14 mmol) is combined with Ph₃P (44 mg, 0.17 mmol) and imidazole (14 mg, 0.21 mmol) in DCM (1 mL). The mixture is chilled to 0° C. and I₂ (43 mg, 0.17 mmol) is added. The mixture is allowed to warm to RT and is stirred overnight. The reaction mixture is concentrated and the residue is subjected to flash chromatography (5 to 50% EtOAc/Hex) to isolate iodide 35a1.

### Step 2:

To a mixture of iodide 35a1 (30 mg, 0.05 mmol) in DMF (0.5 mL) is added 3,5-dimethylpyrrazole (5 mg, 0.06 mmol) and DIPEA (12 μL, 0.07 mmol). The mixture is warmed to 70° C. and is stirred for 2 hours. Tetrahydrofuran (1 mL), MeOH (0.5 mL), NaOH (1 N, 1 mL, 1.0 mmol) and LiOH—H₂O (10 mg, 0.25 mmol) are added and the mixture is stirred at RT for 3 hours. The mixture is concentrated, taken-up in AcOH (2.5 mL), filtered then injected onto a preparative HPLC to isolate compound 1119.

### Example 36A

### Preparation of Compound 1120

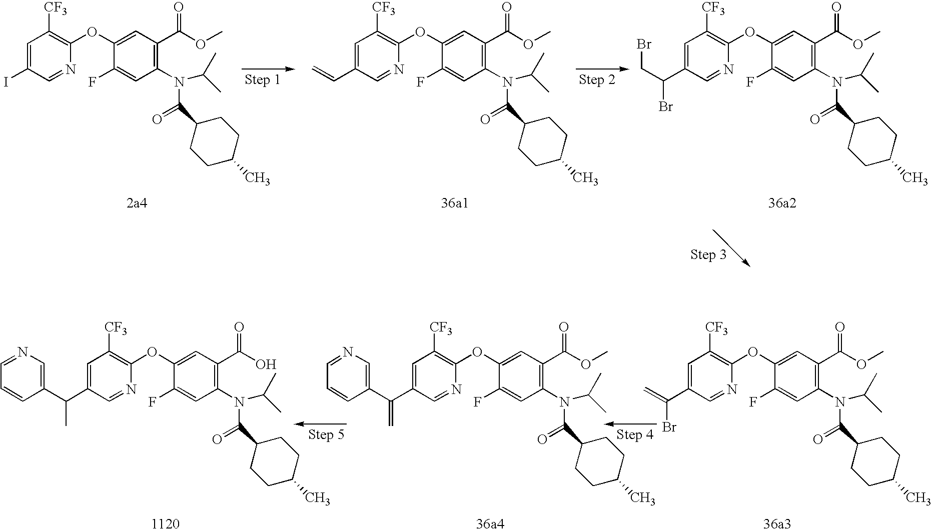

### Step 1:

To a degassed (Ar) mixture of iodide 2a4 (15 g, 24 mmol) in anhydrous THF (300 mL) is added (Ph₃P)₂PdCl₂ (0.84 g, 1.2 mmol) and tributylvinyltin (9.2 g, 29 mmol). The mixture is heated to 70° C. and is stirred overnight. The mixture is concentrated under reduced pressure and the residue is subjected to flash chromatography (5 to 15% EtOAc/Hex) to afford intermediate 36a1.

### Step 2:

To a mixture of alkene 36a1 (350 mg, 0.67 mmol) in anhydrous CHCl₃ (3 mL) is added Br₂ (124 μL, 2.4 mmol). The mixture is stirred for about 2.5 hours at RT before being diluted in EtOAc and washed with water, saturated aqueous NaHCO₃, water and brine. The organic phase is dried with MgSO₄, filtered and concentrated under reduced pressure to afford dibromide 36a2 that is utilized without further purification.

### Step 3:

To a mixture of dibromide 36a2 (400 mg, 0.59 mmol) in anhydrous MeCN (20 mL) is added DBU (132 μL, 0.88 mmol). The mixture is stirred for 15 minutes at RT before being diluted in EtOAc and washed with 10% aqueous citric acid, water, saturated aqueous NaHCO₃, water and brine. The organic phase is dried with MgSO₄, filtered and concentrated under reduced pressure to afford vinylbromide 36a3 that is utilized without further purification.

### Step 4:

Vinylbromide 36a3 is coupled to 3-pyridylboronic acid to form 36a4 using the protocol described in example 14A step 1.

### Step 5a:

Alkene 36a4 is hydrogenated using the protocol described in example 28A step 2.

### Step 5b:

To a mixture of the hydrogenated product (42 mg, 0.07 mmol) in DMSO (1 mL) and water (0.1 mL) is added aqueous NaOH (1 N, 350 μL, 0.35 mmol). The mixture is stirred overnight at RT before being acidified with TFA then injected onto a preparative HPLC to isolate compound 1120.

### Example 37A

### Preparation of Compound 1121

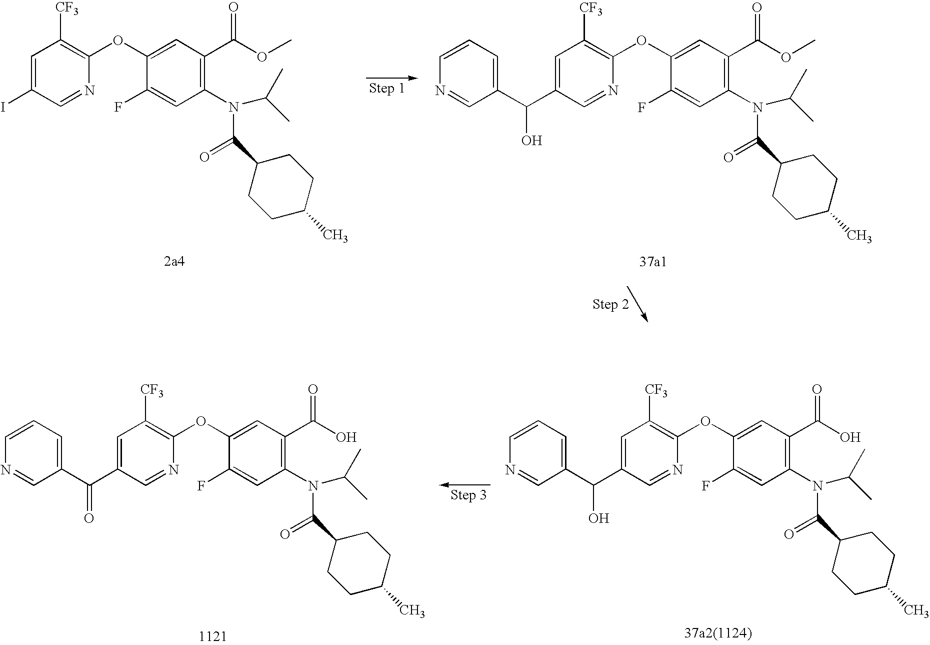

### Step 1:

Reference: Bérillon, L.; Leprêtre, A.; Turck, A.; Ple, N.; Quéguiner, G.; Cahiez, G.; Knochel, P. Synlett 1998, 1359.

To a mixture of iodide 2a4 (250 mg, 0.40 mmol) in anhydrous THF (8 mL) cooled to −40° C. is added i-Pr—MgCl (2 M in THF, 220 μL, 0.44 mmol). The mixture is stirred for 30 minutes at −40° C. before 3-pyridylcarboxaldehyde (57 μL, 0.60 mmol) in THF (0.2 mL) is added. The mixture is stirred for about 2 hours at −40° C. before being allowed to warm to RT. The mixture is diluted in EtOAc and washed with brine. The organic phase is dried with MgSO₄ and filtered. Silica gel is added and the solvent is removed under reduced pressure. The silica gel dry-packed compound is purified by flash chromatography to afford alcohol 37a1.

### Step 2:

Ester 37a1 is saponified to acid 37a2 using the protocol described in example 36A step 5b.

### Step 3:

To a mixture of intermediate 37a2 (64 mg, 0.11 mmol) in DCM (2 mL) is added MnO₂ (190 mg, 2.2 mmol). The mixture is stirred overnight at RT before being filtered through celite (washed with DMC and EtOAc). The filtrate is concentrated under reduced pressure and the residue is taken up in DMSO then injected onto a preparative HPLC to isolate compound 1121.

### Example 38A

### Preparation of Compound 1123

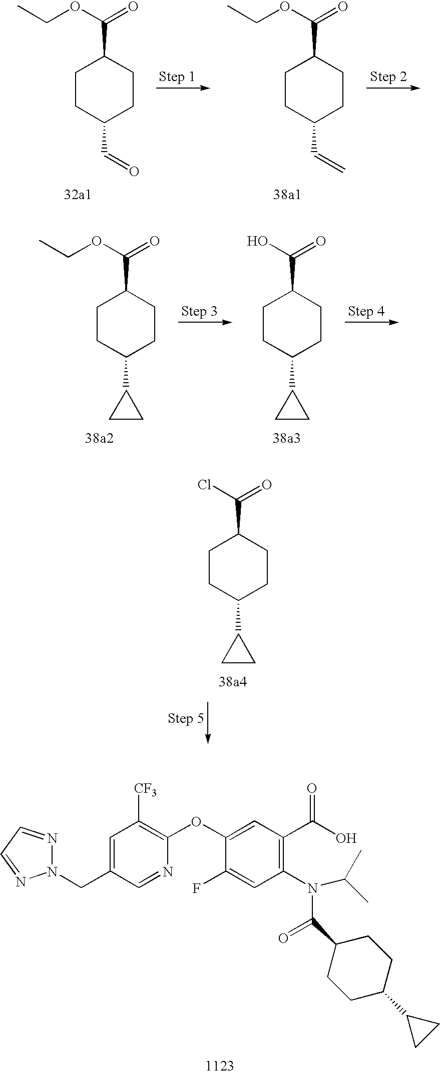

### Step 1:

To a mixture of CH₃P⁺Ph₃Br⁻ (9.9 g, 28 mmol) in anhydrousTHF (200 mL) is added n-BuLi (2.5 M in hexanes, 11.1 mL, 28 mmol). The mixture is stirred for 20 minutes at RT before being chilled to 10° C. Aldehyde 32a1 (4.7 g, 28 mmol) in THF (50 mL) is added. The mixture is allowed to warm to RT and is stirred for 4 hours. The mixture is filtered (washing with THF) and the filtrate is concentrated under reduced pressure. The residue is subjected to flash chromatography (1:8 EtOAc/Hex) to isolate alkene 38a1.

### Step 2:

To a mixture of alkene 38a1 (3.3 g, 20 mmol) in anhydrous DCE (100 mL) cooled to 0° C. is added ZnEt₂ (1.0 M in hexanes, 59 mL, 59 mmol) followed by CH₂ICl (8.6 mL, 118 mmol). The mixture is stirred for about 2 hours at 0° C. then a further 3 hours at RT. The reaction is quenched by the addition of saturated aqueous NH₄Cl and the resulting mixture is partitioned between DCM and water. The organic phase is dried over Na₂SO₄, filtered and concentrated under reduced pressure. The residue is diluted in EtOAc and filtered through a pad of silica gel (washed with EtOAc). The filtrate is concentrated to afford cyclopropane 38a2.

### Step 3:

Ester 38a2 is saponified under the conditions reported in example 31A step 4 to provide acid 38a3.

### Step 4:

The acid 38a3 is converted to acid chloride 38a4 using the conditions described in example 1A step 5.

### Step 5:

The coupling of 38a4 and aniline 17a3 and saponification to produce 1123 is performed as shown in example 27A steps 1 and 3.

### Example 39A

### Method N

### Preparation of Compound 2001

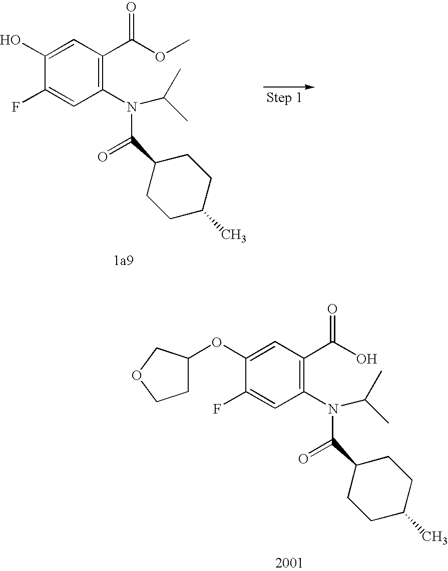

### Step 1a:

To a mixture of phenol 1a9 (30 mg, 0.09 mmol) in THF (1 mL) at RT is added 3-hydroxytetrahydrofuran (12 μL, 0.14 mmol), PPh₃ (35 mg, 0.14 mmol) and DEAD (24 μL, 0.14 mmol). The mixture is stirred for 30 minutes at RT before silica gel is added and the solvent is removed under reduced pressure. The silica gel dry packed compound is purified by combiflash (10 to 60% EtOAc/Hex) to isolate a THF-ether intermediate.

### Step 1b:

The ester intermediate is saponified using the conditions described in example 36A step 5b to produce compound 2001.

### Example 40

### Method O

### Preparation of Compound 2002

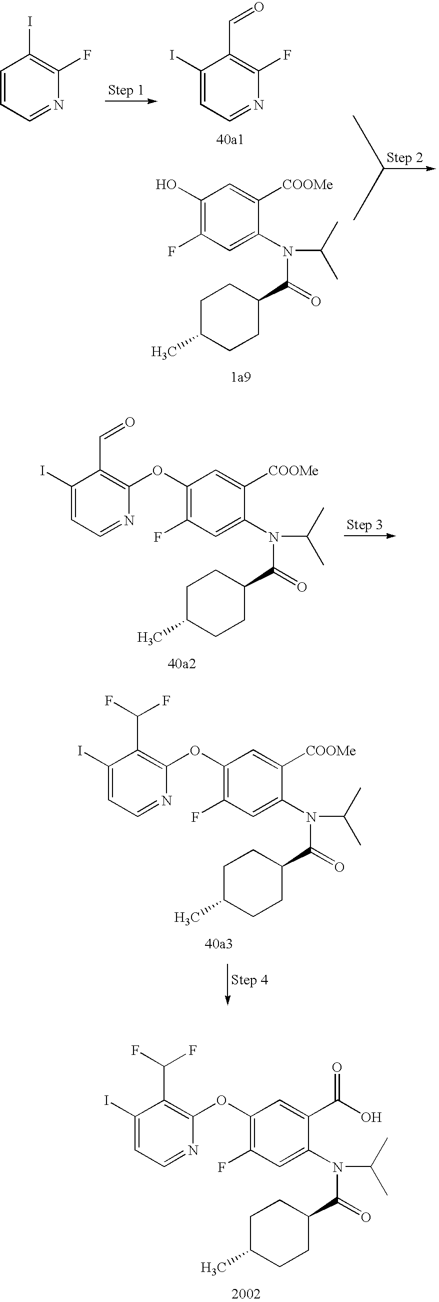

### Step 1:

Reference: Rocca, P.; Cochennec, C.; Marsais, F.; Thomas-dit-Dumont, L.; Mallet, M.; Godard, A.; Queguiner, G. J. Org. Chem. 1993, 58, 7832-7838

LDA is prepared by the drop-wise addition of BuLi (1.6 M, 0.89 mL, 1.4 mmol) to a mixture of diisopropylamine (0.21 mL, 1.5 mmol) in THF (10 mL) at 0° C. The LDA mixture is cooled to −78° C. then slowly added to a mixture of 2-fluoro-3-iodopyridine (300 mg, 1.35 mmol) in THF (5 mL) over 5 minutes. The mixture is stirred for about 1.5 hours at −78° C. then ethylformate (0.12 mL, 1.5 mmol) in THF (1.0 mL) is added. Stirring continues as the mixture is allowed to slowly warm to −50° C. over a period of about 1 hour at which time the reaction is poured into H₂O. The aqueous mixture is extracted (3×) with Et₂O, then the combined organic extracts are washed with brine, dried over MgSO₄, filtered and concentrated under reduced pressure. Purification by flash chromatography (15 to 20% Et₂O in Hex) affords aldehyde 40a1.

### Step 2:

The S_{N}Ar coupling of phenol 1a9 with fluoropyridine 40a1 to produce intermediate 40a2 is performed as described in example 2A step 3.

### Step 3:

To a mixture of compound 40a2 (345 mg, 0.59 mmol) and DCM (1 mL) is added Deoxofluor™ (0.5 mL, 2.7 mmol). The mixture is warmed to 50° C. and is stirred for about 45 minutes before being carefully quenched with saturated aqueous NaHCO₃. The aqueous mixture is extracted (3×) with EtOA,c then the combined organic extracts are dried over MgSO₄, filtered and concentrated under reduced pressure. Purification by flash chromatography affords the difluoromethyl derivative 40a3.

### Step 4:

Compound 40a3 is saponified under conditions described in example 14A step 2 to provide 2002.

### Example 41

### Preparation of Compound 2003

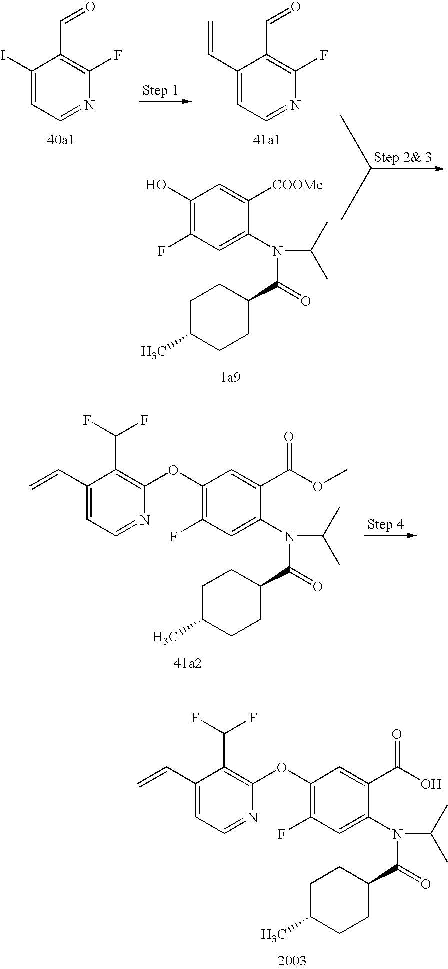

### Step 1:

Aldehyde 40a1 (52 mg, 0.21 mmol) is combined with 4,4,5,5-tetramethyl-2-vinyl-1,2-dioxoborolane (53 μL, 0.31 mmol) and tetrakis(triphenylphosphino)palladium (0) (24 mg, 0.02 mmol) in DMF (2 mL). Aqueous Na₂CO₃ (2.0 M, 0.4 mL, 0.83 mmol) is added then the mixture is heated at 120° C. for 10 minutes. The mixture is diluted in water then extracted (3×) with EtOAc. The combined organic extracts are dried over MgSO₄, filtered and concentrated under reduced pressure. Purification by flash chromatography affords the alkene derivative 41a1.

### Step 2, 3 & 4:

Steps 2, 3 & 4 from example 40 provide compound 2003.

### Example 42

### Method P

### Preparation of Compound 2004

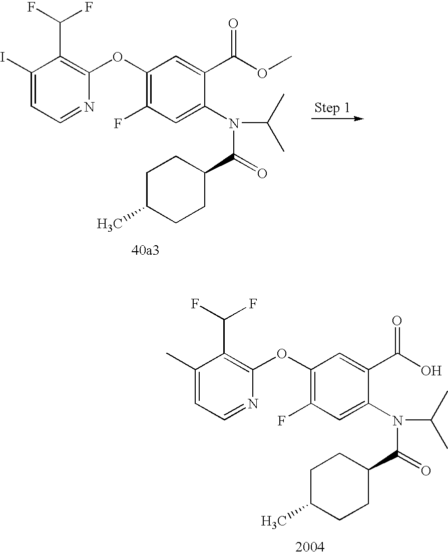

### Step 1:

Reference: Walker, S. D.; Barder, T. E.; Martinelli, J. R.; Buchwald, S. L. Angew. Chem. Int. Ed. 2004, 43, 1871.

Iodide 40a3 (16 mg, 0.03 mmol) is combined with methane boronic acid (2 mg, 0.04 mmol) and bis(tri-tert-butylbutylphosphino)palladium (0) (1 mg, 0.003 mmol) in DMF (1 mL). Aqueous Na₂CO₃ (2.0 M, 30 μL, 0.05 mmol) is added then the mixture is heated at 150° C. for about 12 minutes. The mixture is diluted in EtOAc then washed with water and brine. The organic phase is dried over MgSO₄, filtered and concentrated under reduced pressure.

The crude product is saponified under conditions described in example 14A step 2 to provide compound 2004.

### Example 43A

### Preparation of Compounds 2009 & 2010

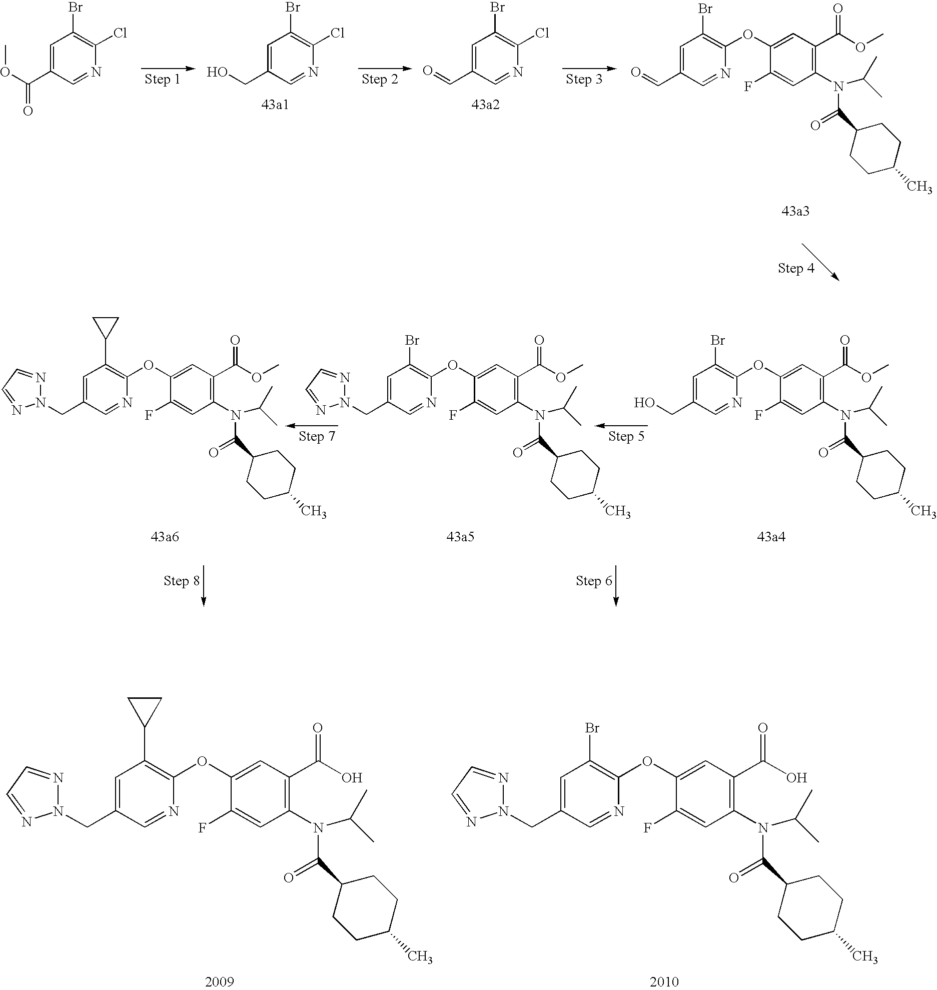

### Step 1:

To a mixture of methyl 5-bromo-6-chloronicotinate (542 mg, 2.2 mmol) in ether (10 mL) chilled to 0° C. is added LiAlH₄ (99 mg, 2.6 mmol). The mixture is allowed to warm to ambient temperature and is stirred overnight. The mixture is poured into saturated aqueous NaHCO₃ and extracted with EtOAc. The organic phase is washed with saturated aqueous NaHCO₃ and brine then dried over Na₂SO₄, filtered and concentrated under reduced pressure. Purification by flash chromatography (50 to 75% EtOAc in Hex) affords alcohol 43a1.

### Step 2:

To a mixture of alcohol 43a1 (352 mg, 1.6 mmol) in DCM (10 mL) chilled to 0° C. is added Dess-Martin periodinane (738 mg, 1.7 mmol). The mixture is stirred at 0° C. for 15 minutes. The mixture is poured into saturated aqueous NaHCO₃ and extracted with EtOAc. The organic phase is washed with saturated aqueous NaHCO₃ and brine then dried over Na₂SO₄, filtered and concentrated under reduced pressure. Purification by flash chromatography (0 to 10% EtOAc in Hex) affords aldehyde 43a2.

### Step 3:

Aldehyde 43a2 (260 mg, 1.0 mmol) is combined with phenol 1a9 (403 mg, 1.2 mmol) and cesium carbonate (442 mg, 1.4 mmol) in DMSO (3 mL). The mixture is heated to 50° C. and is stirred for about 2 hours. The mixture is poured into saturated aqueous NaHCO₃ and extracted with EtOAc. The organic phase is washed with saturated aqueous NaHCO₃ and brine then dried over Na₂SO₄, filtered and concentrated under reduced pressure. Purification by flash chromatography (20 to 90% EtOAc in Hex) affords aldehyde 43a3.

### Step 4:

Reduction of aldehyde 43a3 to alcohol 43a4 is performed as described in example 3A step 3.

### Step 5:

A Mitsunobu reaction as described in example 16A step 2 converts alcohol 43a4 to benzylic triazole 43a5.

### Step 6:

To a mixture of ester 43a5 (40 mg, 0.07 mmol) in THF (0.5 mL) and DMSO (0.2 mL) is added aqueous NaOH (5 M, 150 μL, 0.75 mmol). The mixture is warmed to 50° C. and is stirred for 1 hour. The mixture is acidified with AcOH (0.5 mL) then injected onto a preparative HPLC to isolate compound 2010.

### Step 7:

Bromide 43a5 (100 mg, 0.17 mmol) is combined with tricyclopropylbismuth (90 mg, 0.27 mmol) and K₂CO₃ (47 mg, 0.34 mmol) in DMF (3 mL) in a screw-cap sealed vial. The vial is sparged with Ar for 10 minutes before (Ph₃P)₄Pd (20 mg, 0.02 mmol) is added. The mixture is heated to 100° C. and is stirred for about 2 hours. The mixture is diluted in EtOAc (90 mL) and washed with water (50 mL×3) and brine (50 mL) then dried over Na₂SO₄, filtered and concentrated under reduced pressure. Purification by flash chromatography (20 to 80% EtOAc in Hex) affords cyclopropyl derivative 43a6.

### Step 8:

A saponification as performed in step 6 converts ester 43a6 to compound 2009.

### Example 44

### Preparation of Compounds 2011 & 2012

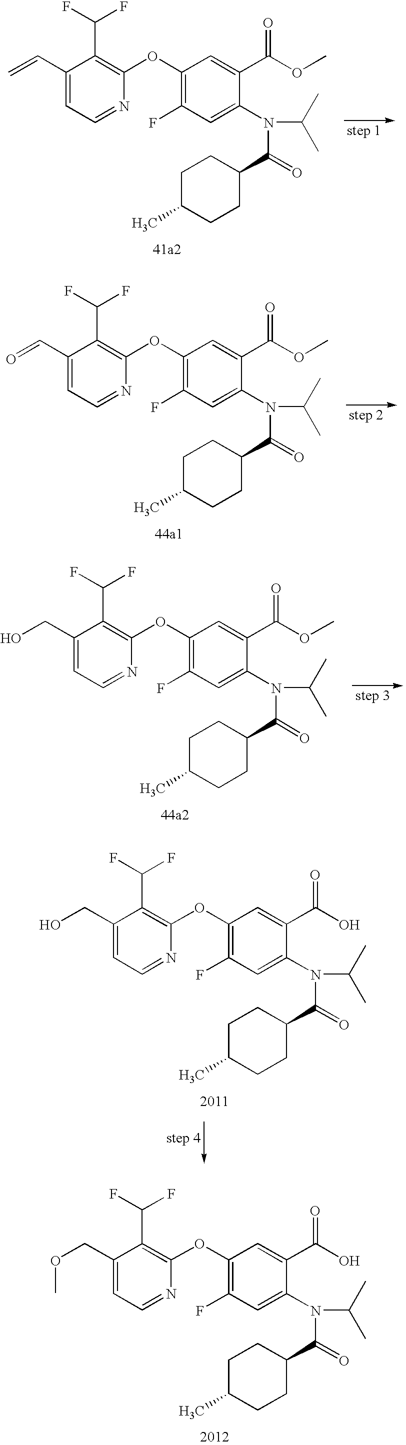

### Step 1:

To a mixture of alkene 41a2 (270 mg, 0.53 mmol) in dioxane (4 mL) and water (2 mL) is added OsO₄ (2.5% in t-BuOH, 540 μL, 0.05 mmol) followed by the portion-wise addition of NaIO₄ (343 mg, 1.6 mmol). The mixture is stirred at RT for 2 days. The reaction mixture is diluted in saturated aqueous Na₂S₂O₃, then extracted with EtOAc (×3). The combined organic extracts are dried over MgSO₄ and filtered. Silica gel is added to the filtrate and the solvent is removed under reduced pressure. The silica gel dry-packed compound is purified by combiflash to afford aldehyde 44a1.

### Step 2:

Aldehyde 44a1 is reduced to alcohol 44a2 under the conditions described in example 3A step 3.

### Step 3:

Intermediate 44a2 is saponified under conditions described in example 14A step 2 to provide 2011.

### Step 4:

To a mixture of alcohol 44a2 (28 mg, 0.05 mmol) in anhydrous THF (1 mL) cooled to −78° C. is added NaHMDS (1.0 M in THF, 65 μL, 0.07 mmol). The mixture is stirred for about 30 minutes at −78° C. before MeI (7 μL, 0.11 mmol) is added and the mixture is allowed to warm to RT. The mixture is stirred for 5 days before MeOH (0.5 mL), water (0.5 mL) and aqueous NaOH (10 N, 11 μL, 0.11 mmol) is added. The reaction mixture is acidified with AcOH, partially concentrated then injected onto a preparative HPLC to isolate compound 2012.

### Example 45

### Preparation of Compound 2014

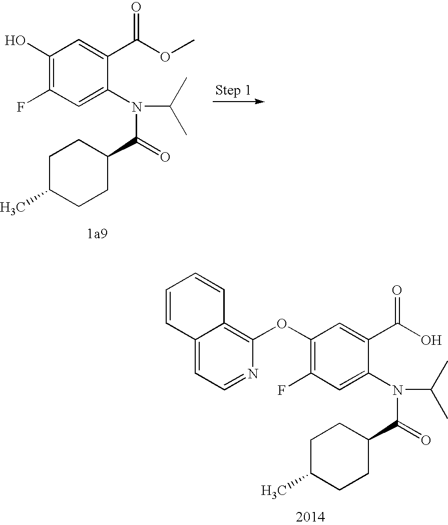

### Step 1:

Phenol 1a9 (100 mg, 0.28 mmol) is combined with 1-chloroisoquinoline (14.4 g, 56.7 mmol) and anhydrous K₂CO₃ (163 g, 1.2 mmol) in DMSO (3 mL). The mixture is heated to 150° C. for 10 minutes in a microwave oven. The reaction mixture is decanted into another vessel and aqueous NaOH (2.5 N, 300 μL, 0.75 mmol) is added. The mixture is stirred for about 3 hours before being acidified with AcOH, filtered then injected onto a preparative HPLC to isolate compound 2014.

### Example 46A

### Method Q

### Preparation of Compound 2015

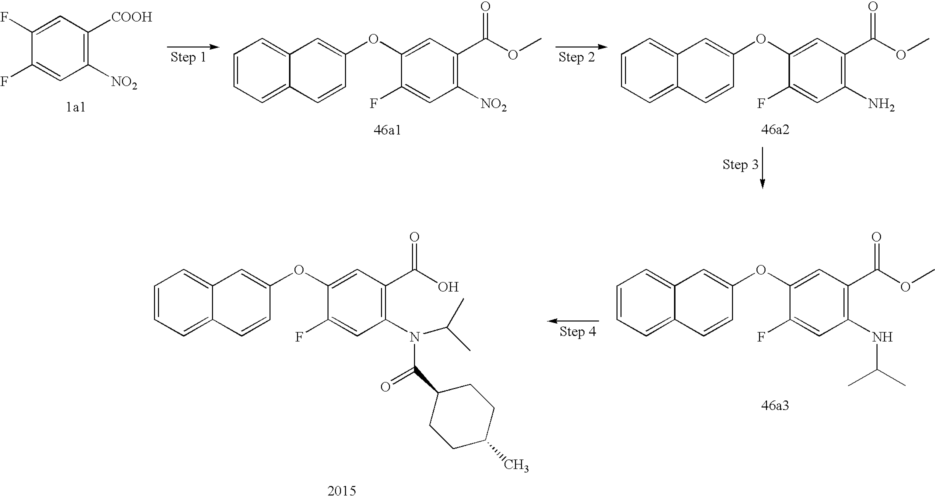

### Step 1:

NaHMDS (1.0 M in THF, 2.0 mL, 2.0 mmol) is added to a solution of 2-naphthol (288 mg, 2.0 mmol) in DMF (5 ml). After 5 minutes, a solution of 4,5-difluoro-2-nitrobenzoic acid 1a1 (200 mg, 0.98 mmol) in DMF (5 mL) is added. The resulting mixture is heated to 80° C. and is stirred for 16 hours. The mixture is diluted in water and extracted with EtOAc/Hex (1:1). The aqueous phase is acidified with 10% aqueous citric acid then extracted with EtOAc. The organic layer is dried with MgSO₄, filtered and concentrated under reduced pressure. The crude compound is taken-up in MeOH then treated with diazomethane (solution in ether) until the characteristic yellow colour persists. The mixture is concentrated under reduced pressure. Diarylether 46a1 is utilized in the next step without further purification.

### Step 2:

The reduction of the nitro arene 46a1 to aniline 46a2 is performed as described in example 28A step 2.

### Steps 3:

The reductive amination described in example 17A step 1 is used to convert aniline 46a2 to N-i-Pr-aniline 46a3.

### Step 4:

Aniline 46a3 (50 mg, 0.14 mmol) is combined with acid chloride 1a7 (67 mg, 0.44 mmol), DMAP (5 mg, 0.04 mmol), anhydrous pyridine (60 μL, 0.74 mmol) in anhydrous DCE. The mixture is heated to 140° C. for 15 minutes in a microwave oven. The reaction mixture is concentrated under reduced pressure. The residue is taken up in DMSO (1 mL) and NaOH (2.5 N, 400 μL, 1.0 mmol) is added. The mixture is stirred for about 2 hours at 45° C. The mixture is acidified with AcOH then injected onto a preparative HPLC to isolate compound 2015.

### Example 47A

### Preparation of Compound 2016

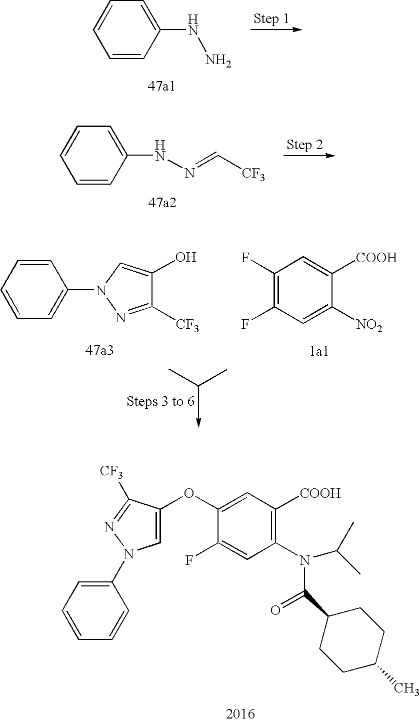

### Step 1:

Reference: Tanaka, K.; Suzuki, T.; Maeno, S.; Mitsuhashi, K. J. Heterocycl. Chem. 1986, 23, 1537.

A mixture of phenylhydrazine 47a1 (500 mg, 4.62 mmol) and 1-ethoxy-2,2,2-trifluoroethanol (667 mg, 4.63 mmol) is heated at 80° C. for 2 h, then cooled and diluted with Et₂O. The mixture is washed with 1N HCl, water and brine, and the organic extract is dried (MgSO₄), filtered and concentrated to give compound 47a2.

### Step 2:

A mixture of aqueous glyoxal (40%, 2.0 g, 13.8 mmol) and n-BuOAc (10 mL) is dried over MgSO₄ and filtered. To the filtrate is added compound 47a2 (853 mg, 4.5 mmol), AcOH (50 μL) and MgSO₄ (462 mg, 3.8 mmol), and the mixture is heated at 120° C. for 6 h. Further glyoxal is added (prepared by extracting aqueous glyoxal (40%, 27 g, 186 mmol) with EtOAc, drying the EtOAc extract over MgSO₄, adding n-BuOAc (10 mL) and concentrating the solution under reduced pressure) and heating at 120° C. is continued for a further 3.5 hours. The mixture is filtered and concentrated, and the residue is mixed with 1N NaOH and washed with CH₂Cl₂. The aqueous phase is acidified to pH 2 with concentrated HCl and extracted three times with CH₂Cl₂. The combined organic extracts are washed with water and brine, dried (MgSO₄), filtered and concentrated. The residue is purified by flash chromatography to provide compound 47a3.

### Step 3 to 6:

Compound 2016 is generated from intermediates 47a3 and 1a1 using the sequence described in Method Q.

### Example 48A

### Preparation of Intermediate 48a3

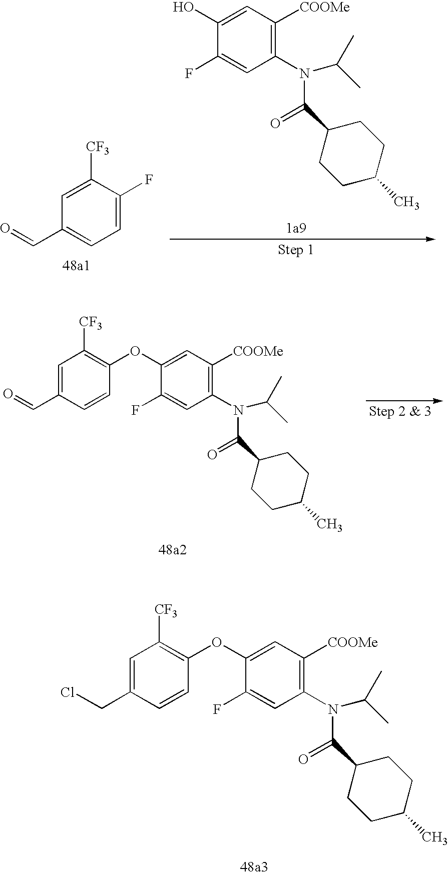

### Step 1:

Phenol 1a9 (14.7 g, 41.83 mmol) is combined with K₂CO₃ (15.3 g, 111 mmol) and 4-fluoro-3-trifluoromethylbenzaldehyde 48a1 (9.6 g, 50 mmol) in DMSO (250 mL). The mixture is heated under Ar at 100° C. and is stirred overnight. The mixture is cooled, diluted with EtOAc and washed with saturated ammonium chloride (2×200 mL) and brine. The organic phase is dried over Na₂SO₄, filtered and the solvent is removed under reduced pressure. Purification by flash chromatography (5 to 25% EtOAc in Hex) affords diarylether 48a2.

### Step 2 & 3:

Aldehyde 48a2 is converted to benzylchloride 48a3 using protocol described in steps 3 & 4 from example 3A.

### Example 49A

### Preparation of Compound 3041

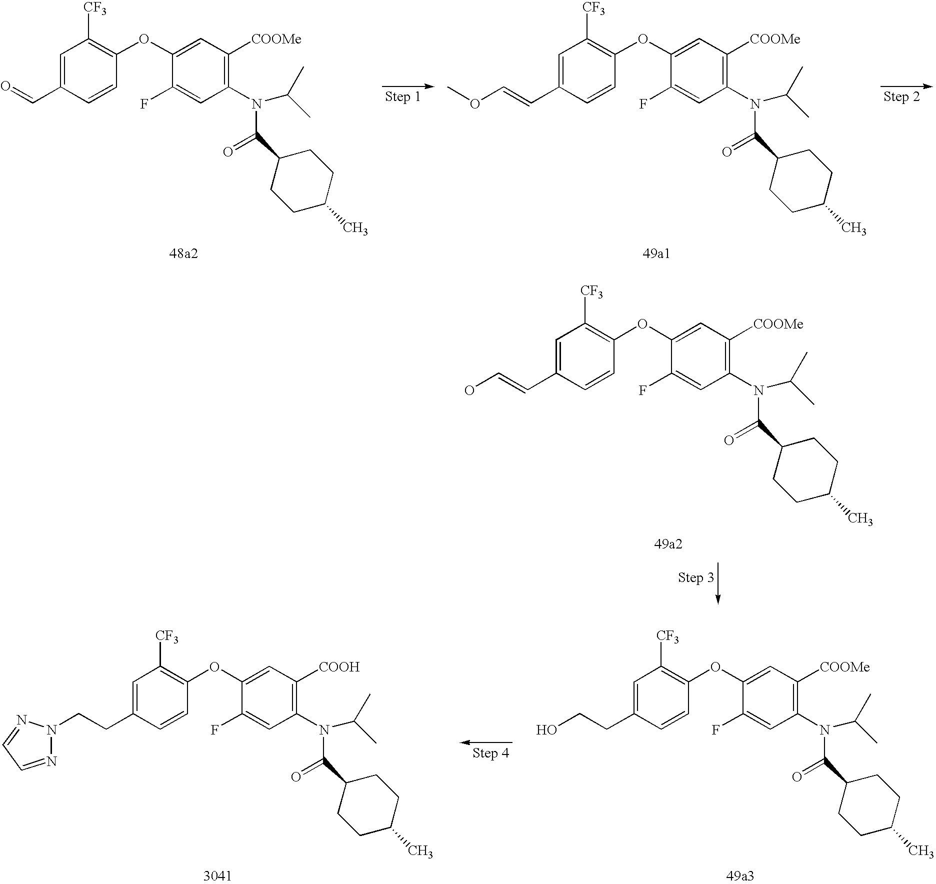

### Step 1:

To a mixture of CH₃OCH₂P⁺Ph₃Cl⁻ (72 mg, 0.21 mmol) in Et₂O at RT is added n-BuLi (1.6 M in hexanes, 130 μL, 0.21 mmol). The mixture is stirred for about 1 hour before aldehyde 48a2 (50 mg, 0.10 mmol) in THF (1 mL) is added drop-wise. Upon completion of the addition, the mixture is warmed to 60° C. and is stirred overnight. The reaction is quenched by the addition of HCl (4.0 M solution in dioxane). The mixture is concentrated then the residue is subjected to flash chromatography (20 to 80% EtOAc/Hex) to isolate enolether 49a1.

### Step 2:

To a mixture of enolether 49a1 (28 mg, 0.05 mmol) in MeOH (1 mL) is added HCl (4.0 M in dioxanes, 1 mL, 4.0 mmol). The mixture is stirred at RT for 1 day before being diluted in water. The aqueous phase is extracted with Et₂O (×3). The combined organic extracts are washed with brine, dried over MgSO₄, filtered and concentrated under reduced pressure to afford aldehyde 49a2.

### Step 3:

Reduction of aldehyde 49a2 to alcohol 49a1 is performed as described in example 3A step 2.

### Step 4:

A Mitsunobu reaction as described in example 16A step 1 followed by saponification as described in example 14A, step 2 provides compound 3041.

### Example 50

### Cell-Based Luciferase Reporter HCV RNA Replication Assay

Compounds of the invention are tested for activity as inhibitors of hepatitis C virus RNA replication in cells expressing a stable subgenomic HCV replicon, using the assay described in WO 2005/028501, herein incorporated by reference. Table 4 lists representative compounds of the invention with their EC₅₀ values and a comparative non-fluorinated analog wherein the presence of the fluorine atom provided an unexpected improvement in cell-based potency.

TABLE 4

| Cpd | EC₅₀ (nM) | Non-fluorinated analog | EC₅₀ (nM) |
|---|---|---|---|
| 1058 | 39.5 | 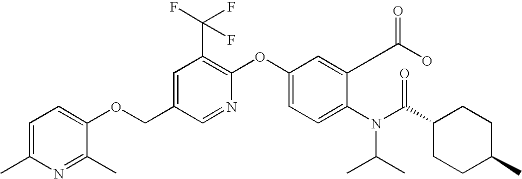 | 86.5 |
| 3016 | 72.5 | 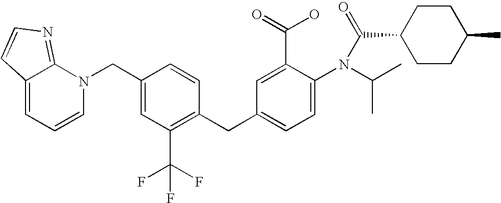 | 207.5 |
| 1042 | 47 | 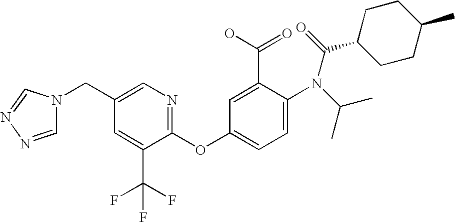 | 160 |
| 1101 | 41.5 | 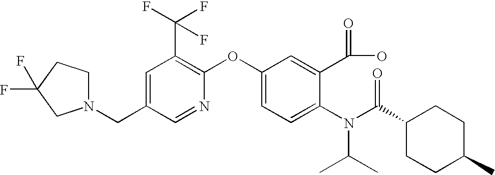 | 89 |
| 3033 | 145 | 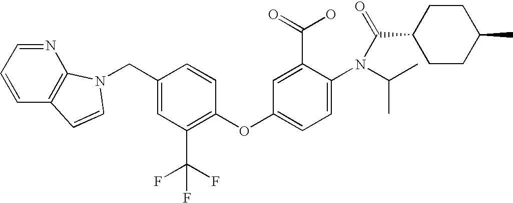 | 265 |

### Example 51

Male human liver microsomes are purchased from Gentest. The pool consisted of microsomes from several donors. The in vitro metabolism in liver microsomes is carried out in a reaction media containing 1 mg of microsomal protein, 2.5 mM NADPH and 2 μM compound in a total volume of 1 ml of 0.066 M Tris buffer, pH 7.4 for 20 minutes at 37° C. Reactions are initiated by the addition of NADPH and terminated at the appropriate times by quenching with an equal volume of a 1:1 mixture of acetonitrile:methanol. The collected samples are centrifuged at 2000 g at 4° C. for 10 minutes and the resulting supernatants are analyzed by HPLC (Waters 600E HPLC controller, Waters 717 Autosampler or Waters Alliance 2695 or Waters Alliance 2795, Waters 996 photodiode array detector, Column: Waters C8 Symmetry (3×150 mm, 5 μm), Solvents: A=acetonitrile, B=water w/50 mM KH₂PO₄, pH 3, Gradient: 5% A:95% B, 8 min linear gradient to 70% A, Flow Rate: 0.7 mL/min

Table 5 lists representative compounds of the invention with their HLM values and a comparative non-fluorinated analog wherein the presence of the fluorine atom provided an unexpected improvement in metabolic stability.

TABLE 5

| Cpd | HLM (min) | Non-fluorinated analog | HLM (min) |
|---|---|---|---|
| 1011 | 136 | 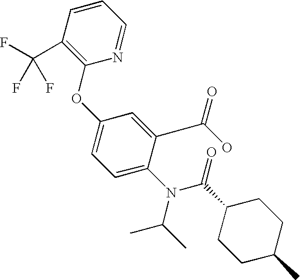 | 69 |
| 1072 | 129 | 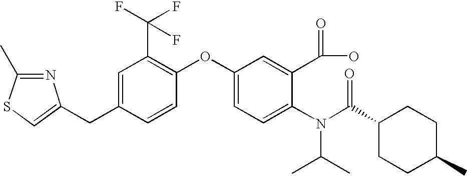 | 29 |

### Tables of Compounds

The following tables list compounds representative of the invention. Retention times (t_{R}) for each compound are measured using the standard analytical HPLC conditions described in the Examples. As is well known to one skilled in the art, retention time values are sensitive to the specific measurement conditions. Therefore, even if identical conditions of solvent, flow rate, linear gradient, and the like are used, the retention time values may vary when measured, for example, on different HPLC instruments. Even when measured on the same instrument, the values may vary when measured, for example, using different individual HPLC columns, or, when measured on the same instrument and the same individual column, the values may vary, for example, between individual measurements taken on different occasions.

TABLE 1

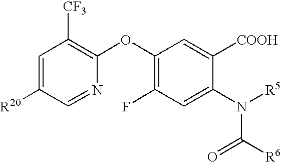

| Cpd | R²⁰ | R⁵ | R⁶ | t_{R} (min) | MS (M + H)⁺ | Method |
|---|---|---|---|---|---|---|
| 1001 | 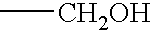 | 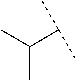 | 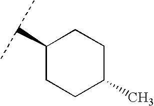 | 6.4 | 513.2 | EX 3A |
| 1002 | 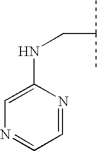 | 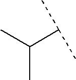 | 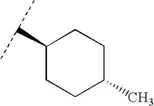 | 6.6 | 590.2 | EX 4AA |
| 1003 | 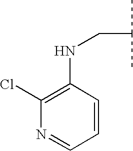 | 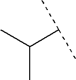 | 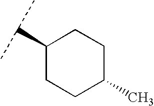 | 7.4 | 623.2 | A |
| 1004 | 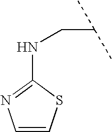 | 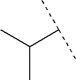 | 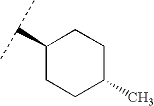 | 5.8 | 595.2 | A |
| 1005 | 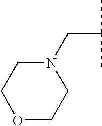 | 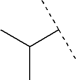 | 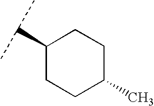 | 5.5 | 582.2 | A |
| 1006 | 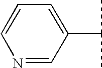 | 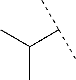 | 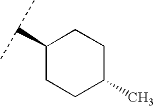 | 5.3 | 560.2 | EX 5AB |
| 1007 | 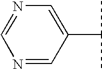 | 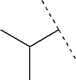 | 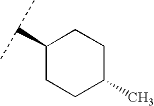 | 6.1 | 561.2 | B |
| 1008 | 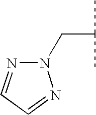 | 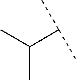 | 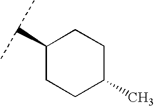 | 6.2 | 564.2 | EX 6A |
| 1009 | 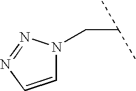 | 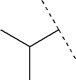 | 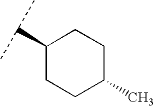 | 5.8 | 564.2 | EX 6A |
| 1010 | 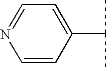 | 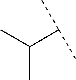 | 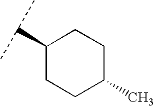 | 5.2 | 560.2 | B |
| 1011 | H | 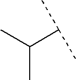 | 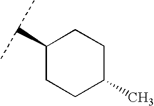 | 5.9 | 483.1 | EX 7A |
| 1012 | 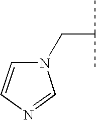 | 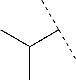 | 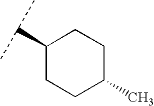 | 5.1 | 563.2 | EX 8C |
| 1013 | 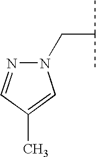 | 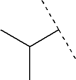 | 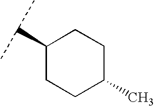 | 7.2 | 577.2 | C |
| 1014 | 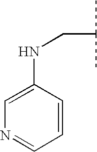 | 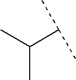 | 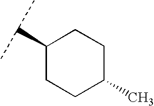 | 5.3 | 589.2 | C |
| 1015 | 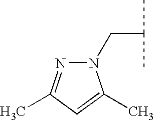 | 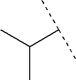 | 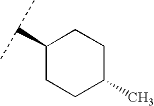 | 7.1 | 591.2 | C |
| 1016 | 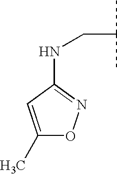 | 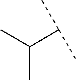 | 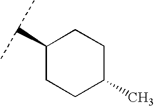 | 7.0 | 593.2 | C |
| 1017 | 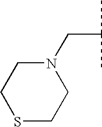 | 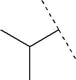 | 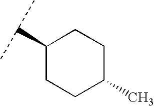 | 5.2 | 598.2 | EX 9D |
| 1018 | 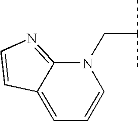 | 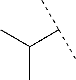 | 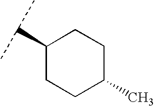 | 5.3 | 613.2 | C |
| 1019 | 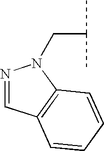 | 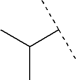 | 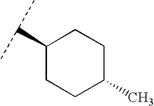 | 7.7 | 613.2 | C |
| 1020 | 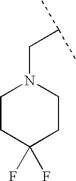 | 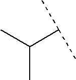 | 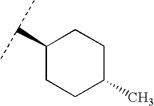 | 5.6 | 616.2 | D |
| 1021 | 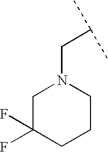 | 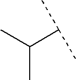 | 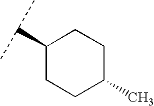 | 5.9 | 616.2 | D |
| 1022 | 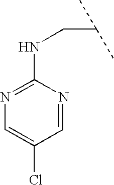 | 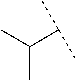 | 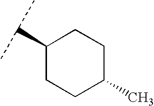 | 7.6 | 624.2 | C |
| 1023 | 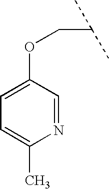 | 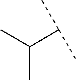 | 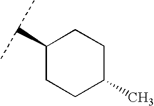 | 5.4 | 604.2 | C |
| 1024 | 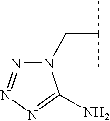 | 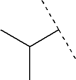 | 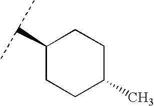 | 6.1 | 580.2 | C |
| 1025 | 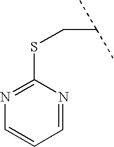 | 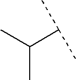 | 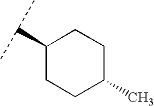 | 7.4 | 607.2 | C |
| 1026 | 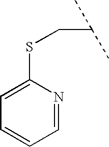 | 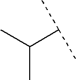 | 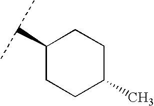 | 7.7 | 606.2 | C |
| 1027 | 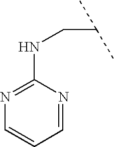 | 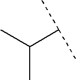 | 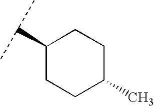 | 6.2 | 590.2 | C |
| 1028 | 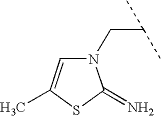 | 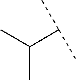 | 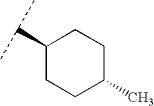 | 5.3 | 609.2 | C |
| 1029 | 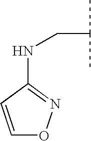 | 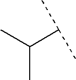 | 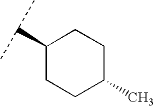 | 6.9 | 579.2 | C |
| 1030 | 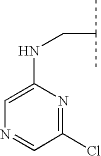 | 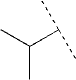 | 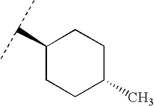 | 7.5 | 624.2 | C |
| 1031 | 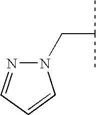 | 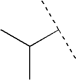 | 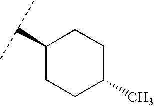 | 7.0 | 563.2 | C |
| 1032 | 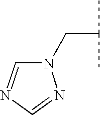 | 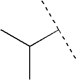 | 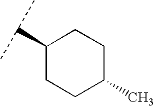 | 6.2 | 564.2 | C |
| 1033 | 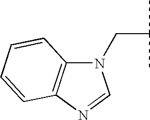 | 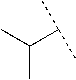 | 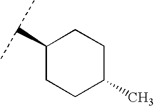 | 5.5 | 613.2 | C |
| 1034 | 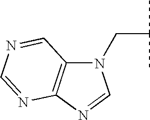 | 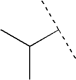 | 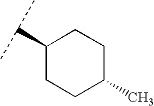 | 5.8 | 615.2 | C |
| 1035 | 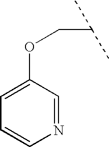 | 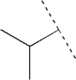 | 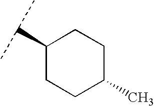 | 6.4 | 590.2 | C |
| 1036 | 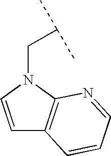 | 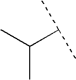 | 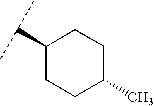 | 7.4 | 613.2 | C |
| 1037 | 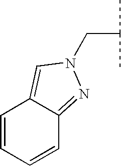 | 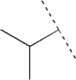 | 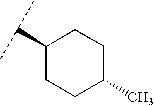 | 7.4 | 613.2 | C |
| 1038 | 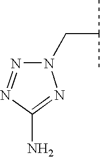 | 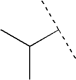 | 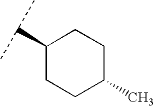 | 6.4 | 580.2 | C |
| 1039 | 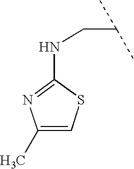 | 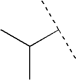 | 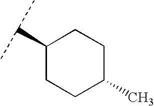 | 5.5 | 609.2 | C |
| 1040 | 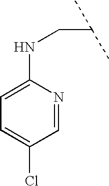 | 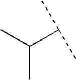 | 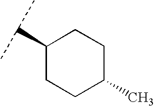 | 7.0 | 623.2 | C |
| 1041 | 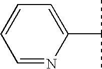 | 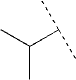 | 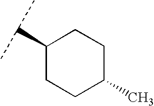 | 6.2 | 560.1 | EX 9 |
| 1042 | 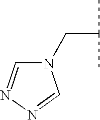 | 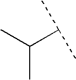 | 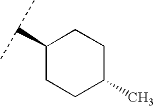 | 5.3 | 564.1 | EX 10 |
| 1043 | 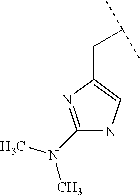 | 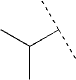 | 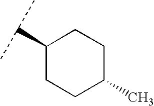 | 5.5 | 623.2 | EX 11AD |
| 1044 | 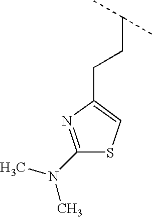 | 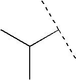 | 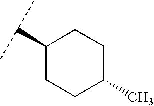 | 5.6 | 637.2 | EX 12AE |
| 1045 | —CH₂OCH₃ | 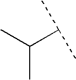 | 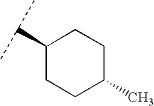 | 7.0 | 527.2 | EX 13A |
| 1046 | 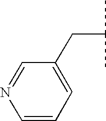 | 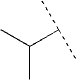 | 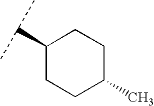 | 5.3 | 574.2 | EX 14AF |
| 1047 | 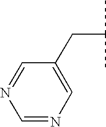 | 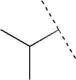 | 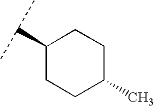 | 5.9 | 575.2 | F |
| 1048 | 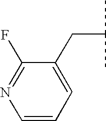 | 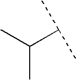 | 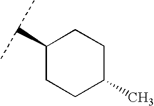 | 6.6 | 592.2 | F |
| 1049 | 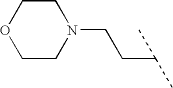 | 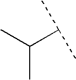 | 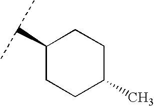 | 4.9 | 596.3 | M |
| 1050 | 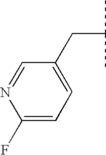 | 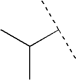 | 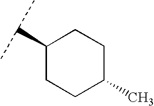 | 6.6 | 592.4 | F |
| 1051 | 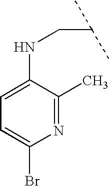 | 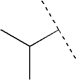 | 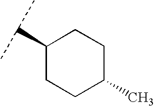 | 8.1 | 681.2 | EX 15AG |
| 1052 | 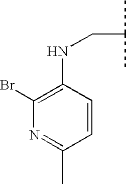 | 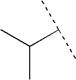 | 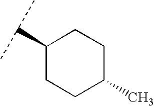 | 8.1 | 681.2 | G |
| 1053 | 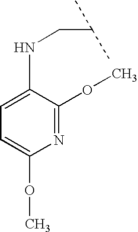 | 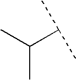 | 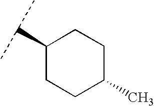 | 8.0 | 649.3 | G |
| 1054 | 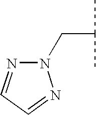 | 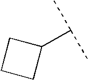 | 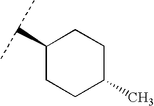 | 7.7 | 576.2 | EX 16A |
| 1055 | 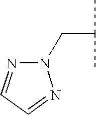 | 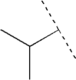 | 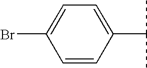 | 5.9 | 620.1 | EX 17AH |
| 1056 | 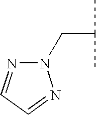 | 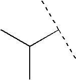 | 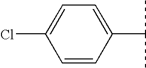 | 6.7 | 578.1 | H |
| 1057 | 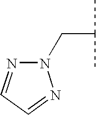 | 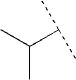 | 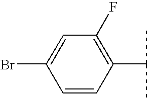 | 6.9 | 640.1 | EX 18A |
| 1058 | 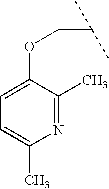 | 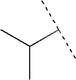 | 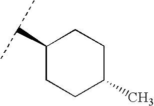 | 5.8 | 618.3 | EX 19AI |
| 1059 | —H | 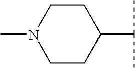 | 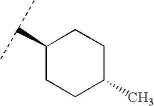 | 5.9 | 538.2 | EX 20 |
| 1060 | 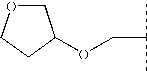 | 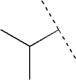 | 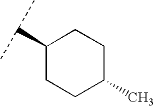 | 7.1 | 583.3 | EX 21A |
| 1061 | 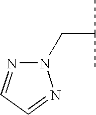 | 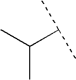 | 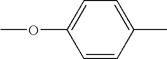 | 6.3 | 574.2 | H |
| 1062 | 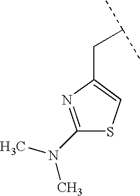 | 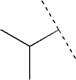 | 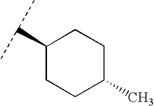 | 6.3 | 637.2 | EX 11AD |
| 1063 | 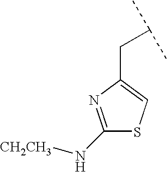 | 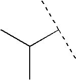 | 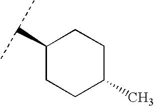 | 6.2 | 623.2 | D |
| 1064 | 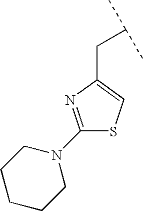 | 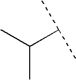 | 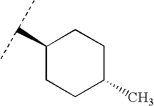 | 6.5 | 663.2 | D |
| 1065 | 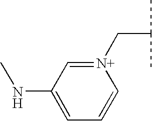 | 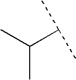 | 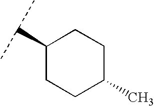 | 5.5 | 603.3 | G |
| 1066 | 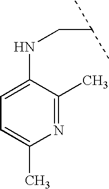 | 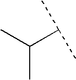 | 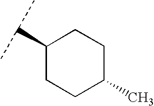 | 5.7 | 617.3 | G |
| 1067 | 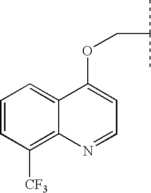 | 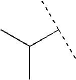 | 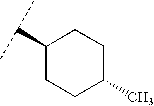 | 8.4 | 708.2 | I |
| 1068 | 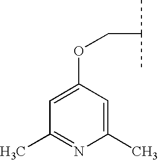 | 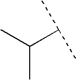 | 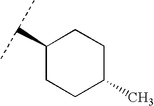 | 5.9 | 618.2 | I |
| 1069 | 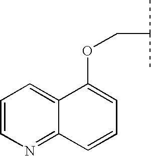 | 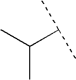 | 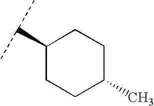 | 6.2 | 640.2 | I |
| 1070 | 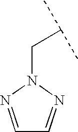 | 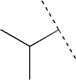 | 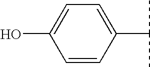 | 5.9 | 560.1 | EX 22A |
| 1071 | 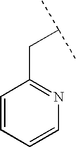 | 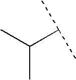 | 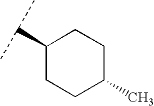 | 6.4 | 574.2 | J |
| 1072 | 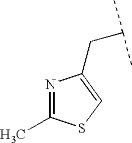 | 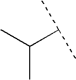 | 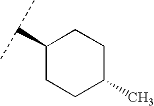 | 7.0 | 594.2 | D |
| 1073 | 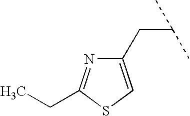 | 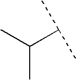 | 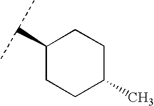 | 7.3 | 608.2 | D |
| 1074 | 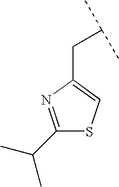 | 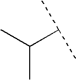 | 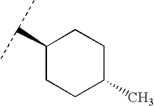 | 7.7 | 622.2 | D |
| 1075 | 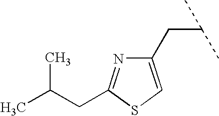 | 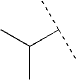 | 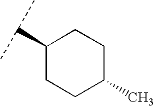 | 7.9 | 636.2 | D |
| 1076 | 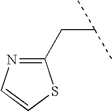 | 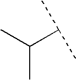 | 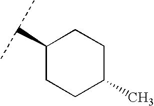 | 7.3 | 580.2 | J |
| 1077 | 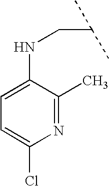 | 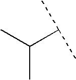 | 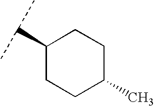 | 8.1 | 637.2 | G |
| 1078 | 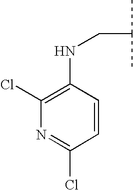 | 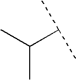 | 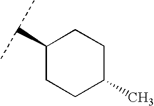 | 7.0 | 657.1 | G |
| 1079 | 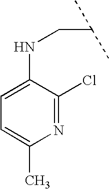 | 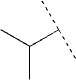 | 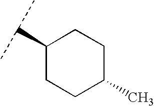 | 8.1 | 637.2 | EX 23AK |
| 1080 | 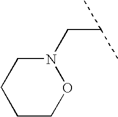 | 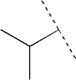 | 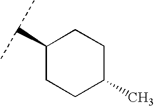 | 7.9 | 580.3 | G |
| 1081 | 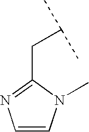 | 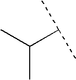 | 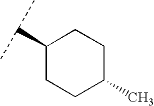 | 5.9 | 577.2 | J |
| 1082 | 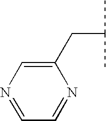 | 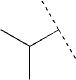 | 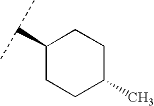 | 7.0 | 575.2 | EX 23AJ |
| 1083 | 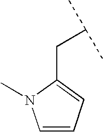 | 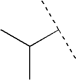 | 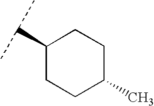 | 7.9 | 576.2 | J |
| 1084 | 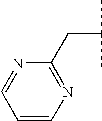 | 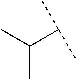 | 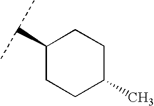 | 7.0 | 575.2 | J |
| 1085 | 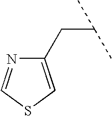 | 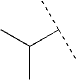 | 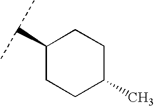 | 7.3 | 580.2 | J |
| 1086 | 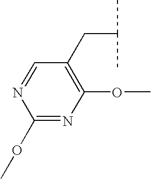 | 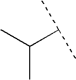 | 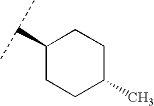 | 7.7 | 635.3 | F |
| 1087 | 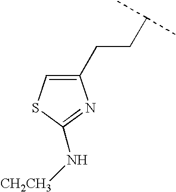 | 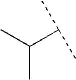 | 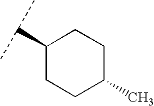 | 5.9 | 637.2 | E |
| 1088 | 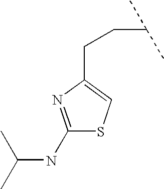 | 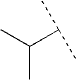 | 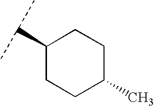 | 6.0 | 651.2 | E |
| 1089 | 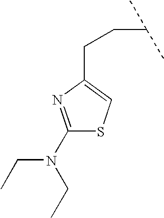 | 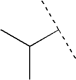 | 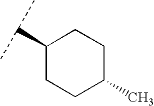 | 6.0 | 665.2 | E |
| 1090 | 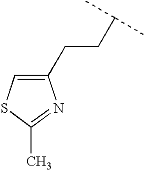 | 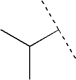 | 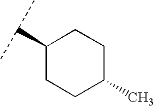 | 6.1 | 608.2 | E |
| 1091 | 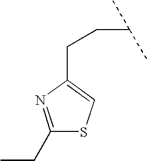 | 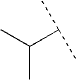 | 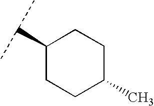 | 6.4 | 622.2 | E |
| 1092 | 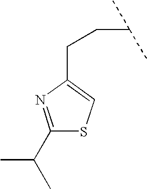 | 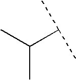 | 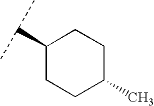 | 6.8 | 636.2 | E |
| 1093 | 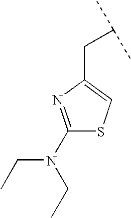 | 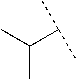 | 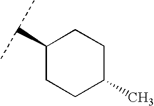 | 6.0 | 651.2 | E |
| 1094 | 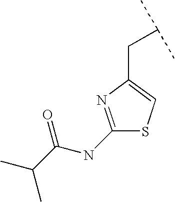 | 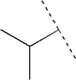 | 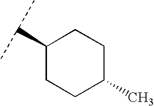 | 6.8 | 665.2 | E |
| 1095 | 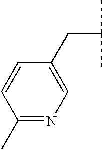 | 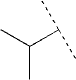 | 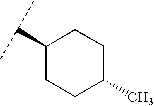 | 5.6 | 588.3 | F |
| 1096 | 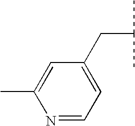 | 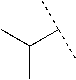 | 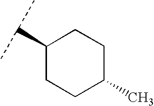 | 5.6 | 588.3 | F |
| 1097 | 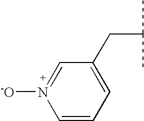 | 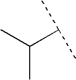 | 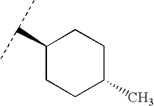 | 6.5 | 590.2 | EX 24A |
| 1098 | 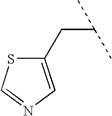 | 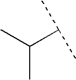 | 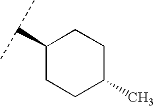 | 7.1 | 580.2 | J |
| 1099 | 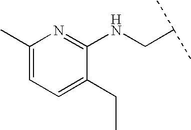 | 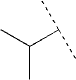 | 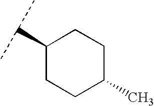 | 5.8 | 631.2 | K |
| 1100 | 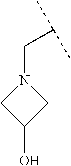 | 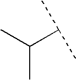 | 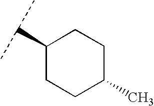 | 4.7 | 568.3 | K |
| 1101 | 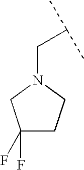 | 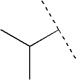 | 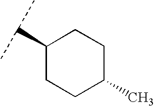 | 6.1 | 602.2 | EX 25A |
| 1102 | 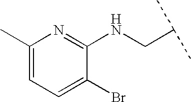 | 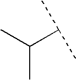 | 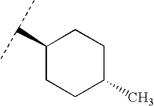 | 8.4 | 681.2 | EX 26A |
| 1103 | 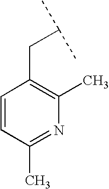 | 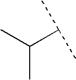 | 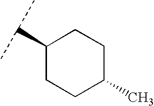 | 5.6 | 602.3 | F |
| 1104 | 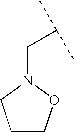 | 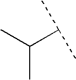 | 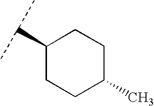 | 6.3 | 568.3 | K |
| 1105 | 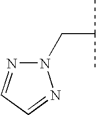 | 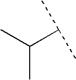 | 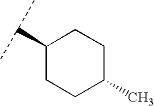 | 7.0 | 550.2 | EX 27A |
| 1106 | 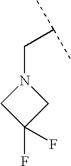 | 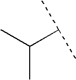 | 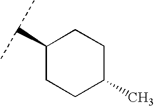 | 5.6 | 588.3 | K |
| 1107 | 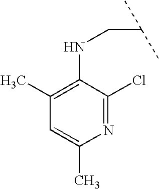 | 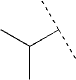 | 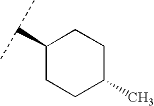 | 6.8 | 651.3 | K |
| 1108 | 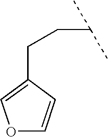 | 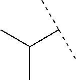 | 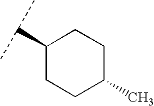 | 7.0 | 577.3 | EX 28AL |
| 1109 | 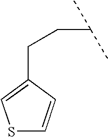 | 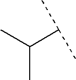 | 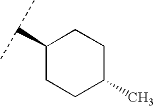 | 7.1 | 593.2 | L |
| 1110 | 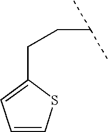 | 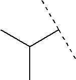 | 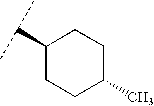 | 7.1 | 593.2 | L |
| 1111 | 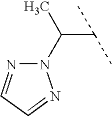 | 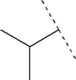 | 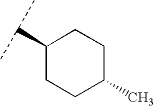 | 6.7 | 578.3 | EX 29A |
| 1112 | 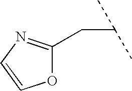 | 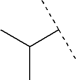 | 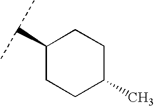 | 7.3 | 564.2 | J |
| 1113 | 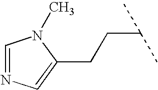 | 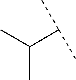 | 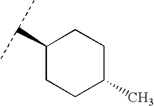 | 5.1 | 591.3 | L |
| 1114 | 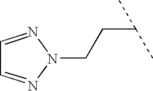 | 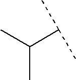 | 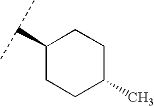 | 6.6 | 578.2 | EX 30A |
| 1115 | 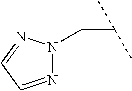 | 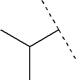 | 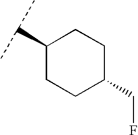 | 7.0 | 582.2 | EX 31A |
| 1116 | 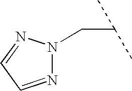 | 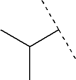 | 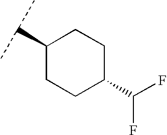 | 7.1 | 600.2 | EX 32A |
| 1117 | 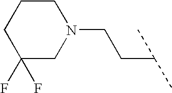 | 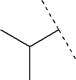 | 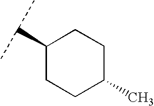 | 5.2 | 630.3 | EX 33AM |
| 1118 | 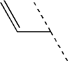 | 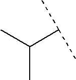 | 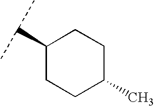 | 6.8 | 509.2 | EX 34A |
| 1119 | 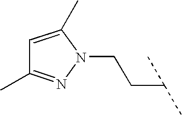 | 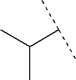 | 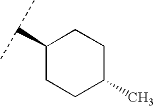 | 6.2 | 605.3 | EX 35A |
| 1120 | 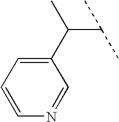 | 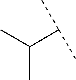 | 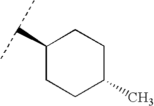 | 5.7 | 588.3 | EX 36A |
| 1121 | 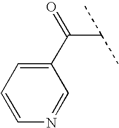 | 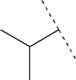 | 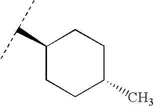 | 6.8 | 588.2 | EX 37A |
| 1122 | 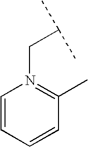 | 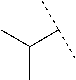 | 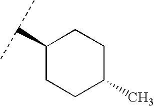 | 5.4 | 588.3 | J |
| 1123 | 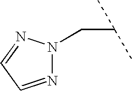 | 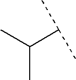 | 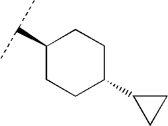 | 8.0 | 590.3 | EX 38A |
| 1124 | 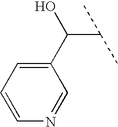 | 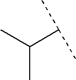 | 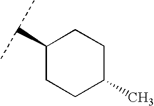 | 5.4 | 590.3 | EX 37A |

TABLE 2

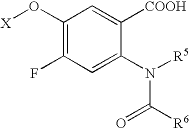

| | | | | t_{R} | MS | |
|---|---|---|---|---|---|---|
| Cpd | X | R⁵ | R⁶ | (min) | (M + H)⁺ | Method |
| 2001 | 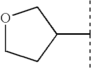 | 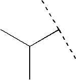 | 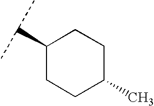 | 5.8 | 408.2 | EX 39A N |
| 2002 | 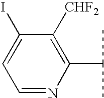 | 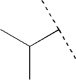 | 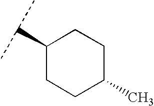 | 7.8 | 591.1 | EX 40 O |
| 2003 | 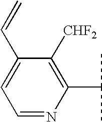 | 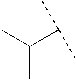 | 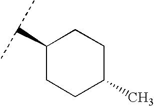 | 7.8 | 491.2 | EX 41 |
| 2004 | 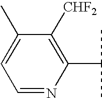 | 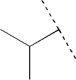 | 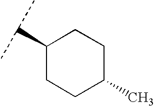 | 7.6 | 479.2 | EX 42 P |
| 2005 | 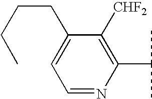 | 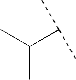 | 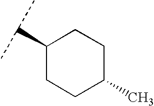 | 8.6 | 521.3 | P |
| 2006 | 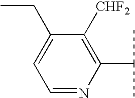 | 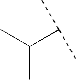 | 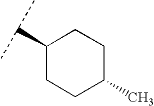 | 7.9 | 493.2 | P |
| 2007 | 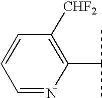 | 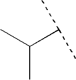 | 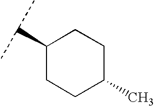 | 7.3 | 465.2 | O |
| 2008 | 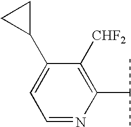 | 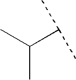 | 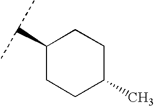 | 7.9 | 503.2 | P |
| 2009 | 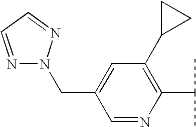 | 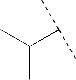 | 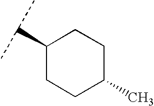 | 7.4 | 536.3 | EX 43A |
| 2010 | 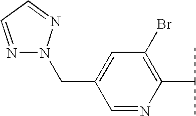 | 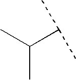 | 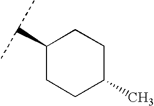 | 7.3 | 574.2 | EX 43A |
| 2011 | 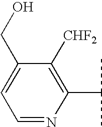 | 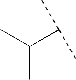 | 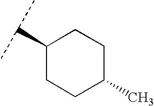 | 6.4 | 495.2 | EX 44 |
| 2012 | 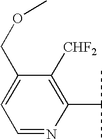 | 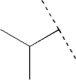 | 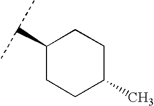 | 7.5 | 509.2 | EX 44 |
| 2013 | 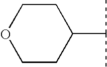 | 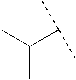 | 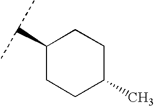 | 6.0 | 422.2 | N |
| 2014 | 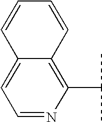 | 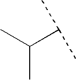 | 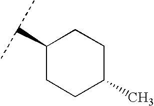 | 6.4 | 465.3 | EX 45 |
| 2015 | 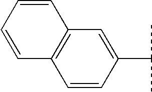 | 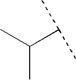 | 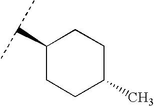 | 8.7 | 464.2 | EX 46A Q |
| 2016 | 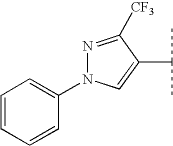 | 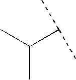 | 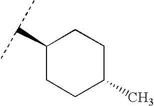 | 6.9 | 548.2 | EX 47A |
| 2017 | 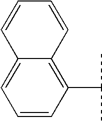 | 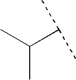 | 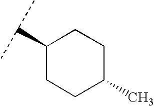 | 6.7 | 464.3 | Q |

TABLE 3

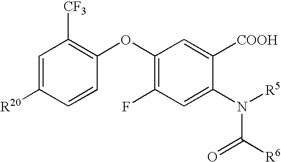

| | | | | t_{R} | MS | |
|---|---|---|---|---|---|---|
| Cpd | R²⁰ | R⁵ | R⁶ | (min) | (M + H)⁺ | Method |
| 3001 | 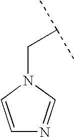 | 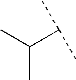 | 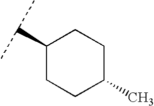 | 5.2 | 562.2 | C |
| 3002 | 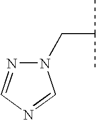 | 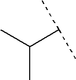 | 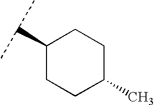 | 6.4 | 563.2 | C |
| 3003 | 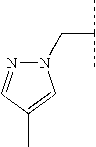 | 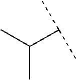 | 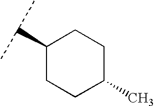 | 7.3 | 576.2 | C |
| 3004 | 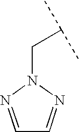 | 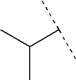 | 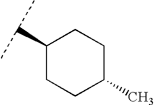 | 7.3 | 563.2 | C |
| 3005 | 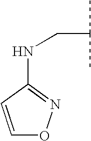 | 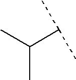 | 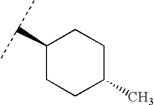 | 7.0 | 578.2 | C |
| 3006 | 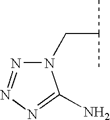 | 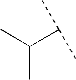 | 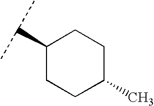 | 6.3 | 579.2 | C |
| 3007 | 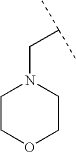 | 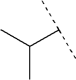 | 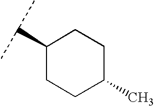 | 5.1 | 581.2 | D |
| 3008 | 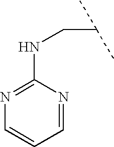 | 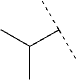 | 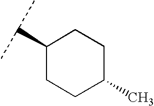 | 6.3 | 589.2 | C |
| 3009 | 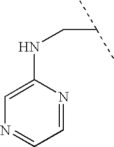 | 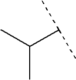 | 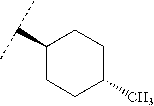 | 5.1 | 589.2 | C |
| 3010 | 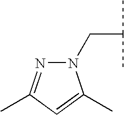 | 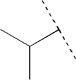 | 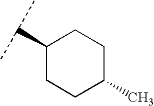 | 7.1 | 590.2 | C |
| 3011 | 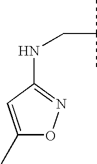 | 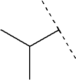 | 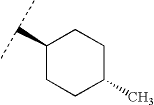 | 7.2 | 592.2 | C |
| 3012 | 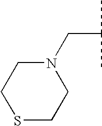 | 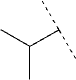 | 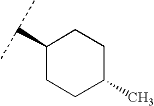 | 5.4 | 597.2 | D |
| 3013 | 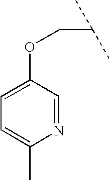 | 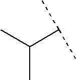 | 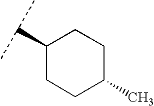 | 5.6 | 603.2 | C |
| 3014 | 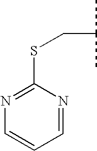 | 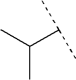 | 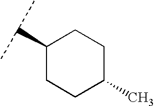 | 7.6 | 606.2 | C |
| 3015 | 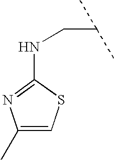 | 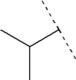 | 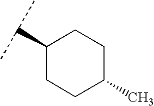 | 5.6 | 608.2 | C |
| 3016 | 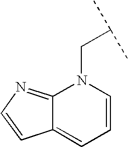 | 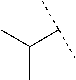 | 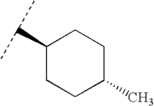 | 5.4 | 612.2 | C |
| 3017 | 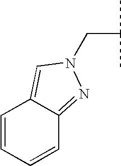 | 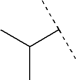 | 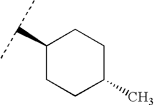 | 7.5 | 612.2 | C |
| 3018 | 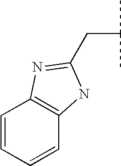 | 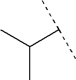 | 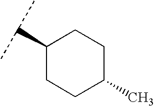 | 5.6 | 612.2 | C |
| 3019 | 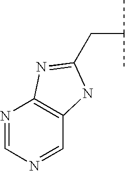 | 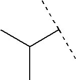 | 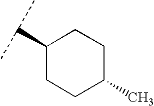 | 6.0 | 614.2 | C |
| 3020 | 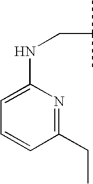 | 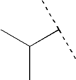 | 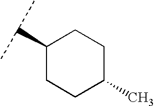 | 5.8 | 616.2 | C |
| 3021 | 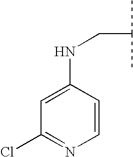 | 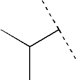 | 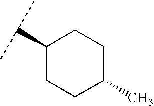 | 5.7 | 622.2 | C |
| 3022 | 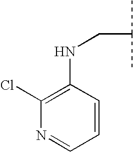 | 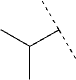 | 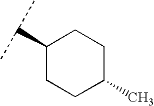 | 7.5 | 622.2 | C |
| 3023 | 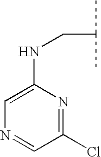 | 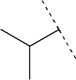 | 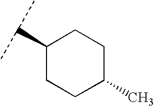 | 7.6 | 623.2 | C |
| 3024 | 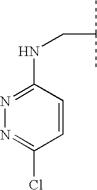 | 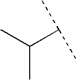 | 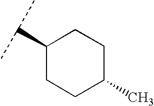 | 5.5 | 623.2 | C |
| 3025 | 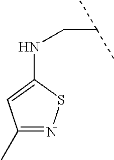 | 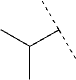 | 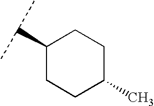 | 6.5 | 608.2 | C |
| 3026 | 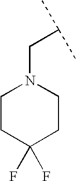 | 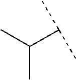 | 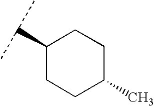 | 5.5 | 615.2 | D |
| 3027 | 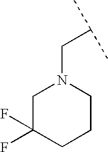 | 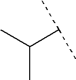 | 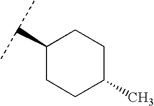 | 5.7 | 615.2 | D |
| 3028 | 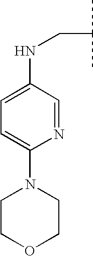 | 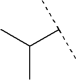 | 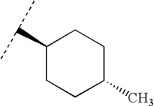 | 5.6 | 673.3 | C |
| 3029 | 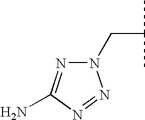 | 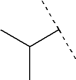 | 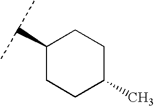 | 6.6 | 579.2 | C |
| 3030 | 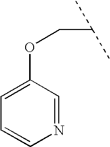 | 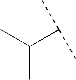 | 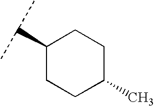 | 5.7 | 589.2 | C |
| 3031 | 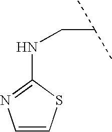 | 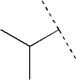 | 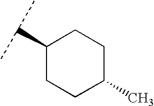 | 5.5 | 594.2 | C |
| 3032 | 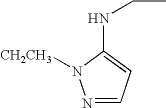 | 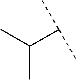 | 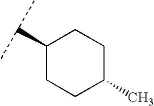 | 5.7 | 605.2 | C |
| 3033 | 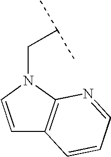 | 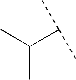 | 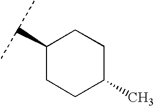 | 7.4 | 612.2 | C |
| 3034 | 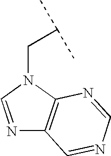 | 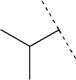 | 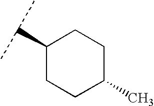 | 6.2 | 614.2 | C |
| 3035 | 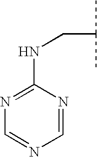 | 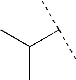 | 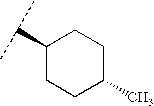 | 6.1 | 590.1 | C |
| 3036 | 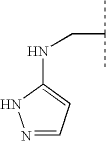 | 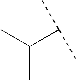 | 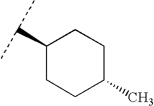 | 5.9 | 577.2 | C |
| 3037 | 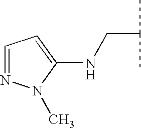 | 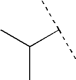 | 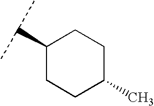 | 6.3 | 591.2 | C |
| 3038 | 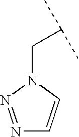 | 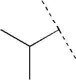 | 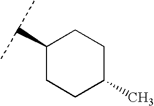 | 6.3 | 563.4 | C |
| 3039 | 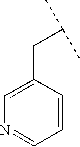 | 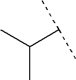 | 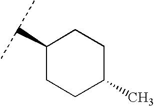 | 6.1 | 573.2 | F |
| 3040 | 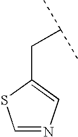 | 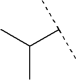 | 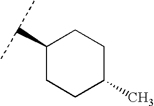 | 7.3 | 579.1 | J |
| 3041 | 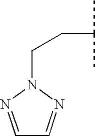 | 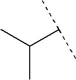 | 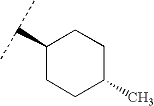 | 6.7 | 577.3 | EX 49A |
| 3042 | 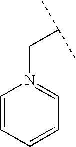 | 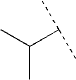 | 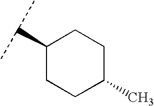 | 5.7 | 573.3 | J |

All of the documents cited herein are incorporated in to the invention as a reference, as if each of them is individually incorporated. Further, it would be appreciated that, in the above teaching of invention, the skilled in the art could make certain changes or modifications to the invention, and these equivalents would still be within the scope of the invention defined by the appended claims of the application.

1-40. (canceled)

41. A compound of formula I: 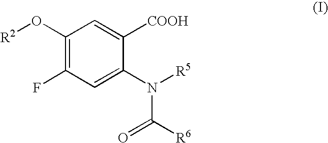
wherein:
R² is aryl or Het, optionally substituted with R²⁰, wherein R²⁰ is 1 to 5 substituents each independently selected from:
a) halo;
b) R⁷, wherein R⁷ is selected from H, (C₁₋₆)alkyl, (C₁₋₆)haloalkyl, (C₃₋₇)cycloalkyl, aryl and Het;
wherein the (C₁₋₆)alkyl and (C₃₋₇)cycloalkyl are optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂
wherein each of the aryl and Het is optionally substituted with 1 to 3 substituents each independently selected from:
i) halo, cyano, oxo, thioxo, imino, —OH, —O—(C₁₋₆)alkyl, —O—(C₁₋₆)haloalkyl, O—(C₃₋₇)cycloalkyl, (C₃₋₇)cycloalkyl, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
ii) (C₁₋₆)alkyl optionally substituted with —OH, —O—(C₁₋₆)haloalkyl, or —O—(C₁₋₆)alkyl; and
iii) aryl or Het, wherein each of the aryl and Het is optionally substituted with halo, (C₁₋₆)alkyl or —O—(C₁₋₆)alkyl;
c) —C(═O)—R⁷, —C(═O)—O—R⁷, —O—R⁷, —S—R⁷, —SO—R⁷, —SO₂—R⁷, —(C₁₋₆)alkylene-R⁷, —(C₁₋₆)alkylene-O—R⁷, —(C₁₋₆)alkylene-S—R⁷, —(C₁₋₆)alkylene-SO—R⁷ or —(C₁₋₆)alkylene-SO₂—R⁷;
wherein R⁷ is as defined above; and wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
d) aryl-(C₁₋₆)alkyl or Het-(C₁₋₆)alkyl,
wherein each of the aryl and Het is optionally substituted with 1 to 3 substituents each independently selected from:
i) halo, cyano, oxo, thioxo, imino, —OH, —O—(C₁₋₆)alkyl, —O—(C₁₋₆)haloalkyl, O—(C₃₋₇)cycloalkyl, (C₃₋₇)cycloalkyl, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —C(═O)—NH(C₃₋₇)cycloalkyl, —C(═O)—N((C₁₋₄)alkyl)(C₃₋₇)cycloalkyl, —NH₂, —NH(C₁₋₄)alkyl, —N((C₁₋₄)alkyl)₂, —NH(C₃₋₇)cycloalkyl, —N((C₁₋₄)alkyl)(C₃₋₇)cycloalkyl or —NH—C(═O)(C₁₋₄)alkyl;
ii) (C₁₋₆)alkyl optionally substituted with —OH, —O—(C₁₋₆)haloalkyl, or —O—(C₁₋₆)alkyl; and
iii) aryl or Het, wherein each of the aryl and Het is optionally substituted with halo, (C₁₋₆)alkyl or —O—(C₁₋₆)alkyl;
wherein the —(C₁₋₆)alkyl portion of the aryl-(C₁₋₆)alkyl or Het-(C₁₋₆)alkyl is optionally substituted with 1 or 2 substituents each independently selected from —OH, —(C₁₋₆)alkyl, halo, —(C₁₋₆)haloalkyl, (C₃₋₇)cycloalkyl, O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂; and
e) —N(R⁸)R⁹, —C(═O)—N(R⁸)R⁹, —SO₂—N(R⁸)R⁹, or —(C₁₋₆)alkylene-N(R⁸)R⁹ wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂;
R⁸ is in each instance independently selected from H, (C₁₋₆)alkyl and (C₃₋₇)cycloalkyl; and
R⁹ is in each instance independently selected from R⁷, —O— (C₁₋₆)alkyl, —(C₁₋₆)alkylene-R⁷, —(C₃₋₇)cycloalkyl-(C₁₋₆)alkyl, —C(═O)—R¹⁰, —C(═O)OR¹⁰ and —C(═O)N(H)R¹⁰;
wherein R⁷ is as defined above;
wherein the —(C₁₋₆)alkylene is optionally substituted with 1 or 2 substituents each independently selected from —OH, —(C₁₋₆)alkyl, halo, —(C₁₋₆)haloalkyl, (C₃₋₇)cycloalkyl, —O—(C₁₋₆)alkyl, cyano, COOH, —NH₂, —NH(C₁₋₄)alkyl, —NH(C₃₋₇)cycloalkyl, —N((C₁₋₄)alkyl)(C₃₋₇)cycloalkyl and —N((C₁₋₄)alkyl)₂
wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
wherein R¹⁰ is in each instance independently selected from (C₁₋₆)alkyl, and Het, wherein said Het is optionally substituted with (C₁₋₆)alkyl;
or R⁸ and R⁹, together with the N to which they are attached, are linked to form a 4- to 7-membered heterocycle optionally further containing 1 to 3 heteroatoms each independently selected from N, O and S, wherein each S heteroatom may, independently and where possible, exist in an oxidized state such that it is further bonded to one or two oxygen atoms to form the groups SO or SO₂;
wherein the heterocycle is optionally substituted with 1 to 3 substituents each independently selected from (C₁₋₆)alkyl, (C₁₋₆)haloalkyl, halo, oxo, —OH, SH, —O(C₁₋₆)alkyl, —S(C₁₋₆)alkyl, (C₃₋₇)cycloalkyl, —NH₂, —NH(C₁₋₆)alkyl, —N((C₁₋₆)alkyl)₂, —NH(C₃₋₇)cycloalkyl, —N((C₁₋₄)alkyl)(C₃₋₇)cycloalkyl, —C(═O)(C₁₋₆)alkyl and —NHC(═O)—(C₁₋₆)alkyl;
R⁵ is selected from H, (C₁₋₆)alkyl, (C₃₋₇)cycloalkyl, (C₃₋₇)cycloalkyl-(C₁₋₆)alkyl and Het;
the (C₁₋₆)alkyl and Het each being optionally substituted with 1 to 4 substituents each independently selected from (C₁₋₆)alkyl, —OH, —COOH, —C(═O)—(C₁₋₆)alkyl, —C(═O)—O—(C₁₋₆)alkyl, —C(═O)—NH—(C₁₋₆)alkyl, —C(═O)—N((C₁₋₆)alkyl)₂, and —SO₂(C₁₋₆)alkyl; and
R⁶ is selected from (C₃₋₇)cycloalkyl and aryl;
the (C₃₋₇)cycloalkyl and aryl each being optionally substituted with 1 to 5 substituents each independently selected from halo, (C₁₋₆)alkyl, (C₁₋₆)haloalkyl, (C₃₋₇)cycloalkyl, —OH, —SH, —O—(C₁₋₄)alkyl and —S—(C₁₋₄)alkyl;
wherein Het is a 4- to 7-membered saturated, unsaturated or aromatic heterocycle having 1 to 4 heteroatoms each independently selected from O, N and S, or a 7- to 14-membered saturated, unsaturated or aromatic heteropolycycle having wherever possible 1 to 5 heteroatoms, each independently selected from O, N and S;
or a salt or ester thereof.

42. The compound according to claim 41 wherein R² is Het wherein Het is a 5- or 6-membered aromatic heterocycle containing 1 or 2 N heteroatoms, wherein Het is optionally substituted with 1 or 2 R²⁰ substituents, wherein R²⁰ is as defined in claim 41.

43. The compound according to claim 42 wherein R² is a group of the formula: 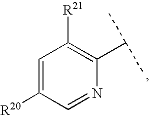
wherein R²¹ is selected from H, halo, (C₁₋₆)alkyl, (C₁₋₆)haloalkyl and (C₃₋₇)cycloalkyl;
and R²⁰ is as defined in claim 42.

44. The compound according to claim 43 wherein R²¹ is CF₃.

45. The compound according to claim 41 wherein R² is a group of the formula: 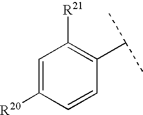
wherein R²¹ is selected from H, halo, (C₁₋₆)alkyl, (C₁₋₆)haloalkyl and (C₃₋₇)cycloalkyl;
and R²⁰ is as defined in claim 41.

46. The compound according to claim 45 wherein R²¹ is CF₃.

47. The compound according to claim 41 wherein R²⁰ is selected from:
b) R⁷, wherein R⁷ is as defined as Het; wherein the Het is optionally substituted with 1 to 3 substituents each independently selected from:
i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
ii) (C₁₋₆)alkyl optionally substituted with —OH, or —O—(C₁₋₆)alkyl; and
iii) Het
c) —C(═O)—R⁷, —(C₁₋₆)alkylene-O—R⁷, —(C₁₋₆)alkylene-S—R⁷,
wherein R⁷ is as defined above;
d) Het-(C₁₋₆)alkyl,
wherein the Het is optionally substituted with 1 to 3 substituents each independently selected from:
i) halo, —OH, (C₁₋₆)haloalkyl, —C(═O)—(C₁₋₆)alkyl, —SO₂(C₁₋₆)alkyl, —C(═O)—NH₂, —C(═O)—NH(C₁₋₄)alkyl, —C(═O)—N((C₁₋₄)alkyl)₂, —NH₂, —NH(C₁₋₄)alkyl, —N((C₁₋₄)alkyl)₂, or —NH—C(═O)(C₁₋₄)alkyl;
ii) (C₁₋₆)alkyl optionally substituted with —OH or —O—(C₁₋₆)alkyl; and
iii) aryl or Het, wherein each of the aryl and Het is optionally substituted with halo or (C₁₋₆)alkyl; and
e) —(C₁₋₆)alkylene-N(R⁸)R⁹, wherein
R⁸ is in each instance independently selected from H and (C₁₋₆)alkyl; and
R⁹ is in each instance independently selected from R⁷, —(C₃₋₇)cycloalkyl-(C₁₋₆)alkyl, —C(═O)—R¹⁰, —C(═O)OR¹⁹ and —C(═O)N(H)R¹⁰;
wherein R⁷ is as defined above;
wherein the (C₁₋₆)alkyl is optionally substituted with 1 or 2 substituents each independently selected from COOH, —NH₂, —NH(C₁₋₄)alkyl, and —N((C₁₋₄)alkyl)₂; and
wherein R¹⁰ is in each instance independently selected from (C₁₋₆)alkyl, and Het, wherein said Het is optionally substituted with (C₁₋₆)alkyl.

48. The compound according to claim 41 wherein R²⁰ is selected from:
c) —(C₁₋₆)alkylene-O—Het, —(C₁₋₆)alkylene-S-Het;
wherein the Het is optionally substituted with 1 to 2 substituents each independently selected from (C₁₋₆)alkyl; and
wherein Het is defined as: 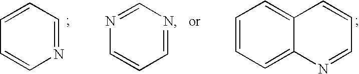
d) Het-(C₁₋₆)alkyl-,
wherein the Het is optionally substituted with 1 to 2 substituents each independently selected from:
i) halo, —OH, —NH₂, —NH(C₁₋₄)alkyl, —N((C₁₋₄)alkyl)₂, or —NH—C(═O)(C₁₋₄)alkyl;
ii) (C₁₋₆)alkyl; and
wherein Het is defined as: 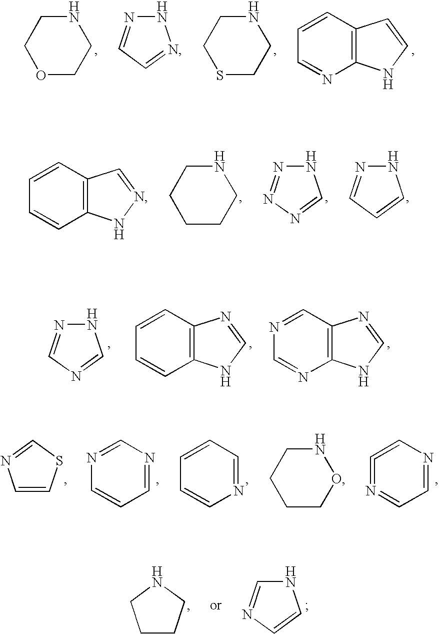
e) —(C₁₋₆)alkylene-N(H)R⁹, wherein
R⁹ is in each instance independently selected from Het, being optionally substituted with 1 or 2 substituents each independently selected from (C₁₋₆)alkyl, halo, O—(C₁₋₆)alkyl, —NH₂, —NH(C₁₋₄)alkyl, and —N((C₁₋₄)alkyl)₂;
and wherein Het is defined as:

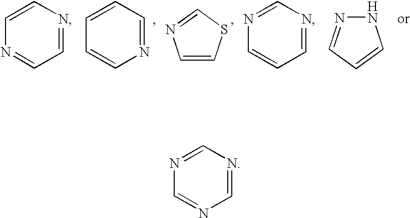

49. The compound according to claim 41 wherein R⁵ is (C₁₋₆)alkyl or (C₃₋₇)cycloalkyl.

50. The compound according to claim 49 wherein R⁵ is 1-methylethyl.

51. The compound according to claim 41 wherein R⁶ is cyclohexyl optionally substituted with 1 to 3 substituents each independently selected from fluoro, (C₁₋₄)alkyl and (C₁₋₄)haloalkyl.

52. The compound according to claim 51 wherein R⁶ is

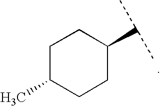

53. A pharmaceutical composition comprising a therapeutically effective amount of a compound according to claim 41, or a pharmaceutically acceptable salt or ester thereof; and one or more pharmaceutically acceptable carriers.

54. The pharmaceutical composition according to claim 53 additionally comprising at least one other antiviral agent.

55. A compound of the formula II: [chemical structure]
